(19) United States
(12) Patent Application Publication
(10) Pub. No.: US 2026/0092269 A1
(43) Pub. Date: Apr. 2, 2026

(54) METHODS FOR SELECTING VARIANT GUIDE RNA AND PRIME EDITING GUIDE RNA SCAFFOLDS, GUIDE RNA AND PRIME EDITING GUIDE RNA COMPOSITIONS AND METHODS OF USING THE SAME

(71) Applicant: Duke University, Durham, NC (US)

(72) Inventors: Bruce SULLENGER, Durham, NC (US); Telmo Llanga, Durham, NC (US); Jonathan ZHOU, Durham, NC (US)

(21) Appl. No.: 19/347,564

(22) Filed: Oct. 1, 2025

Related U.S. Application Data
(60) Provisional application No. 63/701,632, filed on Oct. 1, 2024.
(60) Provisional application No. 63/866,118, filed on Aug. 18, 2025.

(51) Int. Cl. C12N 15/10; C12N 9/22; C12N 15/11; C12N 15/86; C12Q 1/6806; C12Q 1/6869

(52) U.S. Cl. CPC C12N 15/1013; C12N 9/226; C12N 15/1096; C12N 15/111; C12N 15/86; C12Q 1/6806; C12Q 1/6869; C12N 2310/20; C12N 2750/14143

(57) ABSTRACT

Methods of generating novel guide nucleic acids, novel guide nucleic acids generated by the methods, mixtures and complexes comprising the novel guide nucleic acids are disclosed.

### CROSS-REFERENCE TO RELATED APPLICATIONS

This application claims priority to U.S. Provisional Application Nos. 63/701,632 filed on Oct. 1, 2024, and 63/866,118 filed on Aug. 18, 2025, the contents of which are incorporated by reference in their entireties.

### STATEMENT REGARDING FEDERALLY SPONSORED RESEARCH

This invention was made with government support under grant number F99-HG013438 awarded by the National Human Genome Research Institute of the National Institutes of Health. The government has certain rights in this invention.

### REFERENCE TO AN ELECTRONIC SEQUENCE LISTING

The contents of the electronic sequence listing (155554.00795.xml; Size: 2,085,775 bytes; and Date of Creation: Oct. 1, 2025) is herein incorporated by reference in its entirety.

### FILED OF THE INVENTION

The present disclosure relates to gene editing technologies, and more particularly to methods for selecting functional variant guide RNA and prime editing guide RNA scaffolds using SELEX-based approaches to improve editing efficiency at genomic targets. The derived guide RNA and prime editing guide RNA compositions are also provided.

### BACKGROUND

Systematic Evolution of Ligands by EXponential Enrichment (SELEX) is a technique used to generate aptamers, DNA or RNA molecules which bind specifically to a target ligand, by randomly generating an initial pool of candidate sequences and iteratively selecting for candidates with the intended functionality. CRISPR-Cas9 is a gene editing tool which can be engineered to target nearly any location in the genome using a guide RNA (gRNA) which both specifies the target sequence through its spacer domain and binds to a Cas9 endonuclease through its aptameric scaffold domain. However, there remains difficulty in using SELEX selection method to evolve CRISPR-Cas9 gRNA scaffold sequences due to the fact that such scaffold sequences can elicit deleterious self-folding interactions when paired with different sets of genomic targets in a sequence-dependent manner. Hence, there remains a need for new methods to allow for the selection of CRISPR-Cas9 gRNA scaffold sequences.

Prime editing is a CRISPR-Cas9 system which can install specific DNA sequence substitutions, insertions, and deletions without requiring double-stranded breaks or donor DNA using a Cas9 nickase, a reverse transcriptase, and an extended “prime editing gRNA” (pegRNA). pegRNAs include additional material on their 3′ ends to be reverse transcribed directly into the genomic target during prime editing. The addition of this extra RNA sequence can negatively influence pegRNA folding and activity, so there is a need in the art to develop methods of selecting pegRNAs that overcome these limitations to realize the full potential of prime editing.

As disclosed herein the inventors have developed a novel approach using SELEX to evolve and identify functional gRNA and pegRNA scaffolds.

### SUMMARY

In one aspect of the present disclosure, compositions of guide RNA (gRNA) scaffolds are provided. In some embodiments, the gRNA scaffolds comprise SEQ ID NO: 1-134. The compositions may further comprise the Cas proteins to generate ribonucleoprotein (RNP) complexes. Also provided are constructs encoding the gRNAs and optionally encoding the Cas proteins and AAV vectors comprising these constructs.

Another aspect of the present invention provides a method of using the gRNA scaffolds provided herein.

Another aspect of the present invention provides a method of generating a gRNA capable of binding to a Cas protein. In some embodiments, the method comprises, a) generating an RNP complex by combining a Cas protein with a gRNA having a conserved target region and a randomized scaffold region; b) introducing a target DNA bound to or capable of binding to a bead, wherein the DNA comprises a PAM site and sequence complementary to the conserved target region of the gRNA; c) mixing the RNP complex with the bead and target DNA to generate an RNP-bead mixture; d) separating the RNP-bead complex from the mixture, and; e) harvesting the gRNA from the RNP-bead complex.

Another aspect of the present disclosure provides a method of enriching recovery of edited sequences after prime editing comprising: (a) generating a ribonucleoprotein (RNP)-DNA complex by contacting at least one prime editing guide RNA (pegRNA) with a target DNA and a prime editing protein complex comprising a Cas9 nickase and a reverse transcriptase (RT), wherein the pegRNA comprises a 5′ region and a 3′ region complementary to the target DNA and an intended edited region to be incorporated into the target DNA the reverse transcript; (b) incubating the RNP-DNA complex to allow the Cas nickase to generate a nick in the target DNA and the RT to incorporate the complement of the intended edit into the nicked strand of the target DNA to generate a single-strand DNA incorporated edit; (c) introducing a nucleic acid probe sequence complementary to the single-strand DNA incorporated edit to bind to the edited DNA, wherein the probe is labeled or is capable of binding to a label; and (d) separating the edited DNA by selecting for the label and thus enriching for recovery of edited sequences.

In some embodiments, the probe further comprises a homopolynucleotide. In some embodiments, the label is biotin. In some embodiments, the probe comprises at least on locked nucleic acid. In some embodiments, the pegRNA is a pool of pegRNA comprising at least 10 pegRNAs designed to edit the same target DNA and including the intended edited region. In some embodiments, the pegRNA is capable of binding to a modified or engineered Cas protein. In some embodiments the intended edit is selected from the group consisting of at least one nucleotide is changed, an insertion of at least one nucleotide is made, a deletion of at least one nucleotide is made and combinations thereof.

### BRIEF DESCRIPTION OF THE DRAWINGS

Non-limiting embodiments of the present invention will be described by way of example with reference to the accompanying figures.

FIG. 1A-1F. SaBLADE-based gRNA evolution. FIG. 1A) Gel shift analysis of RNP complexes. After 20 minutes of incubation, a native polyacrylamide gel evaluated the complex stability and size of; gRNA alone (lane 1), gRNA and SaCas9 (lane 2), gRNA, Cas9, DNA target and proteinase K (lane 3) and gRNA, SaCas9 and DNA target (lane 4). Complexing the gRNA with SaCas9 and DNA target leads to a substantial gel shift. FIG. 1B) ³²P-radiolabeling strategy used to establish and optimize signal (functional wildtype) to noise (non-functional randomized RNA) ratios of various SaCas9 selection conditions. FIG. 1C) Pulldown of DNA target has 1.5:1 ratio of bead bound signal (WT) to noise (randomized). FIG. 1D) Signal to noise increases to 6:1 when beads are pre-bound to DNA targets for both PAM distal and PAM proximal substrate bound beads. FIG. 1E) Changing wash detergent concentration improves signal to noise ratio to 16:1 by reducing noise (randomized) pulldown. FIG. 1F) Diagram of the SaBLADE selection utilized. Reverse transcription and PCR allow the regeneration of an enriched T7 promoter DNA library, which upon transcription can be used for iterative rounds of selection.

FIG. 2A-2F. SaBLADE yields diverse and potent gRNAs. FIG. 2A) The starting DNA library for gRNA directed evolution contains constant regions at the ends surrounding a variable region randomized with a probability of 58% of the wildtype base at each position, and 14% of each of the other 3 non-wildtype bases. FIG. 2B) Overview of positive selection (PAM proximal enrichment rounds only—denoted P) and Mixed selection (including a PAM distal depletion round—denoted M) strategies. Pool activity is assayed by measuring in vitro cleavage of the DNA target used in selection followed by separation of cleavage products by gel electrophoresis. Substrate DNA and cleavage products generated by various pools using a ratio of 20 RNPs per DNA target and incubated for 1 hour. FIG. 2C) Individual top 2 enriched gRNAs (denoted 1 and 2) from round 6 of positive and mixed selections can effectively cleave DNA while the round 4 top individual sequences cannot (ratio of 10 RNPs per DNA target for 30 minutes). “NT” denotes no gRNA treatment negative cleavage control, “+” denotes wildtype scaffold positive cleavage control. “Ladder” denotes DNA size marker. FIG. 2D) Evaluation of the in vitro cleaving activity of SaBLADE gRNAs at four GFP targets distinct from the original selection target. Instances where SaBLADE gRNAs outperform the wildtype gRNA are marked with a cyan asterisk (cleavage assay conducted with a 1 RNP to 1 DNA substrate ratio for 30 minutes). “P” represents a gRNA enriched from the positive selection strategy, and “M” represents a gRNA enriched from the mixed selection strategy. FIG. 2E) Phylogenetic tree of enriched sequences derived from either positive selection or mixed selection and their nucleotide compositions (SEQ ID NOs: 357-412, in descending order). A consensus sequence for each selection process shows sequence diversity in both cases and a diverse level of base conservation. FIG. 2F) Sequence logo of cleaving variants folded in the canonical secondary structure of SaCas9 shows similar mutation frequencies between paired bases. In 5 instances, variants predominantly depart from the wildtype sequence. Asterisk denotes nucleotides whose preference diverges from the wildtype sequence.

FIG. 3A-3B. SaBLADE-derived gRNAs can improve cellular gene editing efficiency. FIG. 3A) Plasmids containing Positive selection SaBLADE variants were transfected into cells targeting GFP and evaluated via Trace decomposition analysis. Four of these gRNA variants are similarly effective as the wildtype scaffold despite containing up to 18 nucleotide changes. Indels were most concentrated between −3 deletions to +1 insertions for all scaffolds regardless of editing activity. Results were correlated with GFP knockout results determined via flow cytometry (R\hat{ }2=0.84) (FIG. 7A). FIG. 3B) Evaluation of gRNA variants obtained using positive-negative SaBLADE. Flow cytometry evaluation of SaCas9-induced GFP knockout in GFP+HEK293 cells following plasmid transfection and expression (top) of gRNA and SaCas9 or transfection of preassembled gRNA-SaCas9 RNPs (bottom). Several mixed selection gRNA variants achieved improved knockout editing efficiency compared to the wildtype gRNA as did the top two positive selection variants via plasmid-based delivery. Mixed selection variants, but not positive selection variants were capable of outperforming WT activity via lipofection of RNPs. (* P<0.05, **P<0.01, ***P<0.001).

FIG. 4A-4H. SaBLADE-derived gRNAs significantly improve editing at difficult genomic targets. Dot plots for base pair probability matrices comparing variant scaffolds (upper right triangle) to wildtype (lower left triangle) for ERBB2 (FIG. 4A), KIF26B (FIG. 4B) and MBTPS2 (FIG. 4C), orange arrows denote structural changes. Evaluation of editing efficiency of gRNA variants after plasmid transfection via trace decomposition of Sanger sequencing reads at 3 sites: gRNA variant P6 edits FIG. 4D) the ERBB2 gene and FIG. 4E) the KIF26B gene at a statistically significant higher levels than the wildtype gRNA by over 2-fold and 4-fold respectively, while (FIG. 4F) gRNA variants P14 and P5 can significantly enhance editing of the MBTPS2 gene by up to 214% compared to the wildtype gRNA. FIG. 4G) CRISPRESSO2 NGS paired end sequencing distribution of deleted base positions distributed in a bell curve around the predicted cut site for all three targets. Insertion mutations (denoted with brown bar) were focused mostly on the predicted cut site (ERBB2—SEQ ID NO: 413; KIF26B—SEQ ID NO: 414; MBTPS2—SEQ ID NO: 415). FIG. 4H) Insertion deletion (Indel) size heatmap suggests preference for deletions over insertions across all samples regardless of scaffold used.

FIG. 5. To determine whether the original BLADE directed evolution method developed for spCas9 was also effective for selecting saCas9 gRNAs, we radiolabeled both a positive cleaving capable control (wild-type scaffold) and a negative cleaving incapable control (randomized pooled gRNA scaffold), then measured their recovery percentages following BLADE selection. As expected, the pulldown percentages for the Streptococcus Pyogenes (spCas9) wild-type gRNA were substantially higher than those for a spCas9 randomized pool gRNA, indicating a robust signal-to-noise ratio. In contrast, the pulldown percentages for the Staphylococcus Aureus (saCas9) wild-type gRNA did not yield a similarly high signal-to-noise ratio when compared to the randomized pool saCas9 gRNA.

FIG. 6. Evaluation of the in vitro cleaving activity of BLADE2 gRNAs at four GFP targets (A-D) that are distinct from the original selection target. (All combinations tested for FIG. 2D).

FIG. 7A-7B. FIG. 7A) Scatterplot comparing plasmid gene editing assessed via TIDE trace decomposition of Sanger sequencing reads (y-axis) to GFP knockout measured by flow cytometry (x-axis). The dotted line indicates the line of best fit. We found high correlation between both measures of gene editing as evidenced by a high coefficient of determination (R\hat{ }2=0.8498). FIG. 7B) RNP gene editing as measured by GFP knockout percentage revealed positive selection BLADE gRNAs were unable to surpass wildtype gRNA activity.

FIG. 8A-8C. FIG. 8A) RNAfold-predicted minimum free energy structures illustrating how genomic spacers pair with either the canonical wildtype scaffold or SaBLADE gRNA scaffolds. Spacer nucleotides are shown in green, and scaffold nucleotides are shown in blue. Red brackets highlight disrupted wildtype repeat-antirepeat base pairing caused by the spacer, while orange brackets indicate restored repeat-antirepeat folding in SaBLADE variants. FIG. 8B) Minimum free energy structures for MBTPS2, which was tested with two additional BLADE2 scaffolds, P1 and P5. FIG. 8C) Dot plot of base pairing probability matrix for MBTPS2 P1 and P5 (upper triangle) compared to wildtype (lower triangle). Predicted minimum free energy (MFE) are in in kcal/mol.

FIG. 9A-9C. FIG. 9A) Cumulative editing efficiency after plasmid transfection of HEK293 cells was measured by CRISPRESSO2 analysis of paired end next generation sequencing of genomic target amplicon. FIG. 9B) Deletion percentage of total edits was similar across scaffolds FIG. 9C) Insertion percentage of total edits.

FIG. 10. SELEX: Systematic Evolution of Ligands by Exponential Enrichment system diagram. Systematic Evolution of Ligands by EXponential enrichment (SELEX) is a process which begins with a random library of oligonucleotides. A functional assay which is adaptable based on intended function of the evolved sequences is applied to the library. Then a partitioning process separates high-performance sequences from low-performance sequences. Finally, high-performance sequences are reamplified to generate a new enriched library, and the process is repeated.

FIG. 11A-11F. Prime editing (PE) is a CRISPR/Cas9 system in which edit-incorporating DNA is reverse transcribed directly into the target without a double-stranded break or donor DNA, increasing editing fidelity and reducing off-target effects. FIG. 11A shows initial binding of the pegRNA to the nCas (Cas nickase)-RT fusion protein. FIG. 11B shows the nicking of the non-target strand by nCas. FIG. 11C shows the reverse transcription of the template into the target site. FIG. 11D shows the resulting two options of the DNA with a flap containing the edit or not. FIG. 11E shows 3′ flap ligation and 5′ flap cleavage. FIG. 11F shows DNA repair incorporating the edit.

FIG. 12. Is a cartoon showing the intended strategy: biotin-tag the DNA flap to select for pegRNA variants. This cartoon depicts a possible functional assay for SELEX evolution of pegRNAs. In this strategy, in vitro prime editing assays are performed using a pegRNA library and biotin-labeled nucleotides, resulting in RNP-DNA complexes with a biotin-tagged flap. Streptavidin magnetic beads are then used to partition complexes with functional pegRNA variants from those with nonfunctional variants.

FIG. 13. PE products are visible on acrylamide gel—but not at all targets. Targ1: HEK3, Targ2: HEXA, Targ3: HBB, Targ4: PRNP. In vitro prime editing efficiency can be visualized by polyacrylamide gel electrophoresis (PAGE) if a fluorescent Cy5 label is attached to the 5′ nicked strand of DNA target. This allows for visualization of the length of the nicked strand; when no nCas9 is added the strand is at its full length, when nCas9 but no RT is added the strand is nicked to its shortest length, and when both nCas9 and RT are added the strand is nicked and extended to an intermediate length. Targ1 is a control target designated HEK3 where in vitro prime editing is efficient. However, at therapeutic targets HEXA, HBB, and PRNP, prime editing is much less efficient

FIG. 14. Addition of biotinylated nucleotides inhibits nCas9 activity. Addition of biotinylated nucleotides inhibits nCas9 activity. However, activity is rescued by pre-nicking with nCas9 before adding RT and biotinylated nucleotides.

FIG. 15. Biotin-labeled PE flap cannot be recaptured at any target. To test whether biotin-labeled prime edited complexes can be partitioned from nonfunctional complexes, in vitro prime editing reactions were performed and then isolated via streptavidin magnetic bead pulldown, after which flow cytometry was used to assess fluorescence of individual beads. No increased fluorescence was detected at any of the prime editing targets compared to the negative bead control, indicating that the biotin-labeled prime edited flap cannot be accessed and partitioned by magnetic beads.

FIG. 16. pegRNAs can be reengineered to insert polyC or polyA flaps, toggling the number of biotinylated C nucleotides. PegRNAs were reengineered to insert polyC or polyA flaps to test 2 alternative strategies: one in which an entire flap of biotinylated C nucleotides was inserted, and one in which a biotinylated oligo(dT) probe was used to bind to a polyA flap

FIG. 17. Prime edited poly A insert can be pulled down via biotin-oligo(dT) probe. Prime edited polyA insert can be pulled down and partitioned using a biotinylated oligo(dT) probe, but not with a polyC flap of biotinylated nucleotides.

FIG. 18. Efficient capture of PE complexes is target-dependent. Partitioning with the polyA insert strategy is effective at the HEK3 control target, but much less effective at the therapeutic targets HEXA, HBB, and PRNP

FIG. 19. Custom-made complementary biotin—oligo probe can be used to pull down WT prime edited insert. Longer probes are more effective—despite the fact that the PE insert is only 10 bp, the 25 bp probe (d25) significantly outperforms the 15 bp probe (d15).

FIG. 20. Updated overview of selection process.

FIG. 21. RNA can be recovered from beads via phenol chloroform extraction. To test whether pegRNA variants can be recovered from magnetic bead partitioned prime edited complexes, phenol chloroform extraction was performed to separate complexes from beads and DNAse was added to remove the DNA target. PAGE and visualization shows that RNA can be recovered.

FIG. 22. Initially we tested Positive saCas9 SELEX reported in WO2022197727A9 for Activity correlation after one round of enrichment (single target).

FIG. 23. Novel Method: Cut Bound Complexes are distinguished by Gel Shift (single target).

FIG. 24. High-Throughput Sequencing of gRNAs is performed from cut out gel bands.

FIG. 25. Cut to Uncut Ratio correlates with Cell CRISPR GFP Knockout Activity.

FIG. 26. Gel Shift for Multiplex Targets.

FIG. 27. Cut Bound Complexes are distinguished by Gel Shift (multiple targets).

FIG. 28. Multiple Targets: Variant Scaffold Activity Levels vs Wildtype. Violin Plots denote activity distribution of variant scaffolds. Dot denotes Wildtype Scaffold Activity.

FIG. 29. Variant Scaffolds Activity Trends. Violin Plots denote activity distribution of variant scaffolds. “0” mutations violin plot denotes wildtype activity range, highlighted range denotes wildtype activity range in bottom graph

FIG. 30A-30C. General strategy of selecting prime editing guide RNA (pegRNA) scaffolds. FIG. 30A Shows strategy 1—biotinylating probe mating prime edited (PE) insert FIG. 30B shows this strategy leads to high background due to probe nonspecific binding to unedited DNA which usually only have 1 to 3 nucleotide differences between the unedited DNA and prime edited insert. Peak indicated as “2” (+RT+Cas9) should have a higher fluorescence than −RT (3) and −Cas9 (4) (background peaks). FIG. 30C shows that redesigning PE insert to include a polyA “barcode” can reduce background. Specific pulldown using a polyT probe matched to a polyA barcode is viable—however, inclusion of this barcode might introduce noncanonical base interactions reducing applicability of results.

FIG. 31A-31B. FIG. 31A shows strategy 2—Terminal transferase (TdT) introduces the polyA “barcode”. TdT is an enzyme which adds nucleotides indiscriminately to free 3′ ends such as the flap generated after prime editing. FIG. 31B This strategy produces high signal and low background as demonstrated by re-amplification of the library after selection round 1. 8-10 cycles of PCR (C8, C10) is sufficient for imageable reamplification indicating high signal. 16 cycles of PCR in −RT control (−RT C16) shows no signal, indicating low background. TdT has several drawbacks-1) TdT strategy is selective for variants that cut, but does not discriminate between those that cut and those that edit since polyA “barcoding” can happen regardless of whether the PE insert is added after the DNA is cut; 2) Additional steps are needed in each round to prepare the pool before and after TdT incubation, leading to lower overall pool retention after each round; and 3) TdT is not used with prime editing in practice, and selection results may be biased towards variants that work better with TdT.

FIG. 32A-32C. Strategy 3—biotinylated LNA probe. FIG. 32A shows that general strategy design. FIG. 32B shows LNA probes have significantly reduced background binding compared to (strategy 1) normal probes. 1=+RT+Cas9, 2=−Cas9, 3=Negative control. FIG. 32C shows simulations of round 1 using LNA probes show high pool recovery with low background recovery. Simulated round 1 at target HEK3 using LNA probe LNA1 shows strong RNA pool recovery for (+Cas) sample and low background recovery in (−Cas) sample, with (RNA) and (DNA) samples as size controls

### DETAILED DESCRIPTION

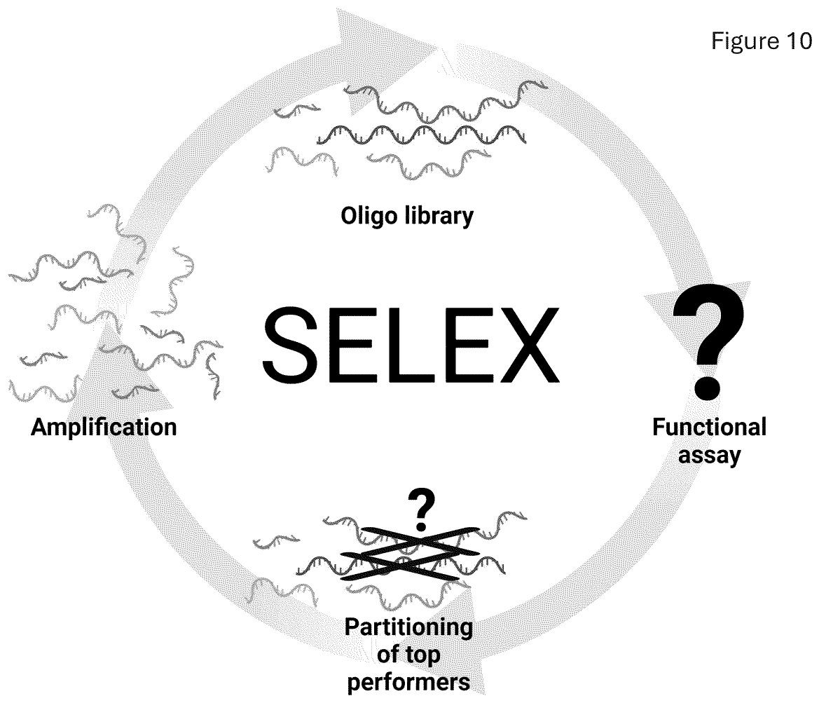

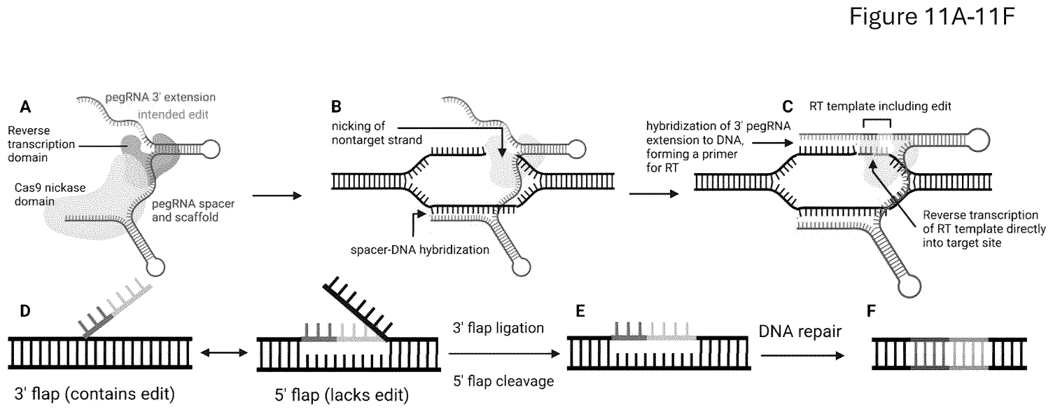

The present disclosure is based, in part, on the discovery by the inventors of a SELEX selection method (See FIG. 10) to evolve new CRISPR-Cas9 gRNA scaffold sequences which demonstrate significant variation from the canonical sequence and can be used in place of the canonical sequence in CRISPR-Cas9 gene editing systems to efficiently cleave and edit DNA in vitro. These variant sequences outperform the canonical sequence at a subset of genomic targets and can rescue functionality at sites that are traditionally difficult to edit using CRISPR-Cas9 systems. The variation from the canonical sequences described herein was unexpected as the extent that gRNAs could be modified was unknown. Disclosed herein are compositions of guide RNA (gRNA) and prime editing gRNA (pegRNA) scaffolds and methods of making and using the same.

CRISPR (clustered regularly interspaced short palindromic repeats) loci are found in a wide range of bacteria and have now been shown to be transcribed to generate a family of targeting RNAs specific for a range of different DNA bacteriophage that can infect the bacterium. In bacteria that express a type II CRISPR/Cas system, these phage-derived sequences are transcribed along with sequences from the adjacent constant region to give a CRISPR RNA (crRNA) which forms a complex with the invariant trans-activating crRNA (tracrRNA), using sequence complementarity between the tracrRNA and an invariant region of the crRNA. This heterodimer, referred to as a guide RNA (gRNA), is then bound by the effector protein of the type II CRISPR/Cas systems, called Cas9. Cas9 has the ability to directly recognize a short DNA sequence called a protospacer adjacent motif (PAM). In the case of the commonly used Streptococcus pyogenes (Sp) Cas9 protein, the PAM site is 5′-NGG-3′. The Cas9 protein scans a target genome for the PAM sequence and then binds and queries the DNA for full 5′ sequence complementarity to the variable part of the crRNA. If detected, the Cas9 protein directly cleaves both strands of the target bacteriophage DNA˜3 bp 5′ to the PAM, using two distinct protein domains: the Cas9 RuvC-like domain cleaves the non-complementary strand, while the Cas9 HNH nuclease domain cleaves the complementary strand. This dsDNA break then induces the degradation of the phage DNA genome and blocks infection of the bacterium. Thus CRISPR/Cas based systems are both highly specific and allow retargeting to new genomic loci with variable efficiencies.

A key step forward in making the Cas systems more user-friendly for genetic engineering in human cells was the demonstration that the crRNA and tracrRNA could be linked by an artificial loop sequence to generate a fully functional small guide RNA (sgRNA)˜100 nt in length. Further work, including mutational analysis of DNA targets, has revealed that sequence specificity for Cas9 relies both on the PAM and on full complementarity to the 3′˜13 nt of the ˜20 nt variable region of the sgRNA, with more 5′ sequences making only a minor contribution. Cas9 therefore has an ˜15 bp (13 bp in the guide and 2 bp in the PAM) sequence with specificity for targeting DNA. The inventors previously disclosed novel methods of generating CRISPR genome editing agents using a combinatorial chemistry approach for Streptococcus pyogenes Cas9, as described in WO2022197727A9, which is incorporated by reference in its entirety. This system makes use of the relatively stable binding of the Cas9 protein and gRNA to the target DNA to isolated functional gRNAs with evolved sequences.

CRISPR systems have been identified and characterized from many different bacteria and any of these Cas enzymes may be used in the methods described herein, for example, Cas9, Cpf1, Cas3, Cas8a-c, Cas10, Cas13, Cas14, Cse1, Csy1, Csn2, Cas4, Csm2, Cm5, Csf1, C2c2, CasX, CasY, Cas14, and NgAgo. The Cas protein can be from any bacterial or archaeal species. For example, in some embodiments, the Cas protein is from Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis Corynebacter diphtheria, Acidaminococcus, Lachnospiraceae bacterium, or Prevotella. For example Cas9 proteins from any of Corynebacter, Suiterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter may be used. In some embodiments, the Cas proteins have modified function e.g., Cas nickase or catalytically dead Cas. In some embodiments, the Cas proteins are fused to another proteins which uses the CRISPR system to be targeted to a specific locus on DNA or RNA. In some embodiments, the Cas protein may be selected based on the cleavage pattern, repair bias, dissociation, PAM recognition or protein size. For example, a specific Cas protein may be selected for use in the present disclosure based on if it dissociates by releasing the DNA after cleavage, allowing for the enzyme to function as a multiple turn over enzyme, or if it remains bound after cleavage, making it a single turnover nuclease.

While experimental dissociation data has not been produced, SaCas9 closely related orthologs with high Cas9 protein sequence identity and high gRNA sequence and structural identity are known. For these, it has been published that these potentially could use a common gRNA scaffold in PMCID: PMC9710800. These include Staphylococcus haemolyticus (Sha2Cas9, 1058 aa, 63.2% identity), Staphylococcus microti (SmiCas9 1064aa, 58.4% identity), Staphylococcus petrasii (SpeCas9, 1058, 63.5% identity). Staphylococcus warneri (SwaCas9, 1054aa, 64.5% identity), Staphylococcus warneri2 (Swa2Cas9, 1054aa, 64.3% identity). Additionally, 16 other Orthologs with high identity include “Staphylococcus equorum Cas9 (SeqCas9, 1053 aa, 97.1% identity Staphylococcus lugdunensis Cas9 (SlugCas9, 1054 aa, 63.2% identity), Staphylococcus epidermidis Cas9 (SepCas9, 1099 aa, 64.2% identity), Staphylococcus haemolyticus Cas9 (ShaCas9, 1055 aa, 63.2% identity) and Staphylococcus lutrae Cas9 (SlutrCas9, 1054 aa, 59.1% identity)” (see also Wang S, et al. Identification of SaCas9 orthologs containing a conserved serine residue that determines simple NNGG PAM recognition. PLoS Biol. 2022 Nov. 30; 20(11):e3001897. doi: 10.1371/journal.pbio.3001897. Erratum in: PLoS Biol. 2025 Mar. 13; 23(3):e3003036. doi: 10.1371/journal.pbio.3003036. PMCID: PMC9710800).

Closely related orthologs of spCas9 include Streptococcus dysgalactiae (Sdy2Cas9, 1367aa, 86.03% identity), Streptococcus phocae (SphCas9,1368 aa, 69.76% identity), Streptococcus equinus strain NCTC12969 (SeqCas9, 1377 aa, 65.83% identity), Streptococcus lutetiensis 033 (Slu2Cas9, 1373 aa, 61.24% identity) (see Liu J. et al. Enhanced genome editing with a Streptococcus equinus Cas9. Commun Biol. 2025 Feb. 7; 8(1):196. doi: 10.1038/s42003-025-07593-z. PMID: 39920233; PMCID: PMC11806022.)

In the Examples, the inventors reduce to practice the novel methods with both Streptococcus pyogenes (Sp) and Staphylococcus aureus (Sa) CRISPR Cas9 systems, but other CRISPR systems may be used. As discussed above, Cas9 proteins rely on a distinct recognition site or PAM. The PAM for Sp Cas9 is 5′-NGG-3′, for Neisseria meningitides (Nme) it is 5′-NNNNGATT-3′ and for Staphylococcus aureus (Sa) the PAM is identified herein as 5′-NNGRRT-3′, where R is purine. Each has a distinct sgRNA scaffold sequence making up the 3′ portion of the single guide RNA. The length of the target sequence specific 5′ portion of the sgRNA varies between the Cas9 enzymes as well. SpCas9 uses 18-20 nucleotide target sequences. NmeCas9 and SaCas9 use an 18-24 nucleotide target sequence.

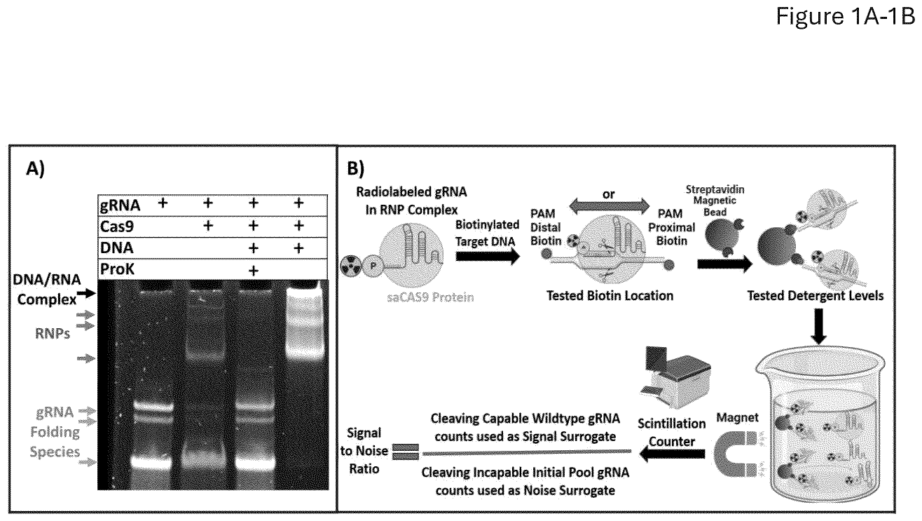

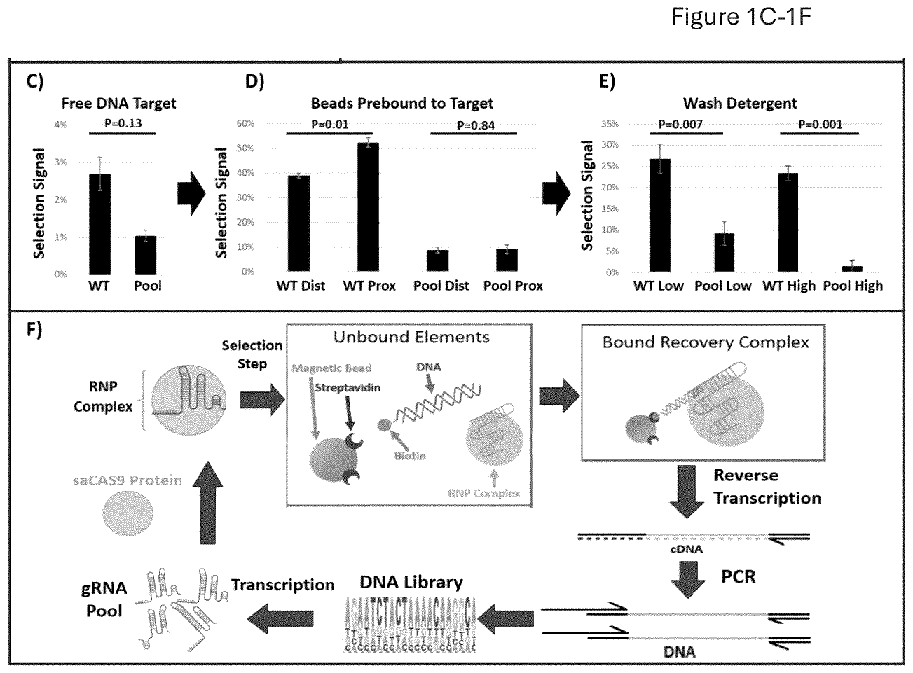

In the CRISPR system, the Cas9 enzyme is directed to cleave the DNA target sequence by the sgRNA (interchangeably referred to as the gRNA). The sgRNA includes at least two portions having two functions. The first portion is the DNA targeting portion of the sgRNA and it is at the 5′ end of the sgRNA relative to the second portion. The first portion of the sgRNA is complementary to a strand of the target sequence, referred to herein as a “template-conserved target complementary region”. The target sequence is immediately 5′ to the PAM sequence for the Cas9 on the target nucleic acid. Thus, the template conserved target complementary region is proximate to the PAM site, i.e., within less than 5 nucleotides, less than 4 nucleotides, less than 3 nucleotides, less than 2 nucleotides, 1 nucleotide away from the PAM site, or the template-conserved target complementary region may comprise the PAM site. As used herein, PAM proximal refers to the right of the cut, which contains the PAM, whereas PAM distal refers to the left of the cut. See FIG. 1B. The portion of the sgRNA that is complementary to the target sequence may be 10 nucleotides, 13 nucleotides, 15 nucleotides, 18 nucleotides, 20 nucleotides, 22 nucleotides or 24 nucleotides in length or any number of nucleotides between 10 and 30. The portion of the sgRNA complementary to the target sequence should be able to hybridize to the sequences in the target strand and is optimally fully complementary to the target sequence. The exact length and positioning of the complementary portion of the sgRNA will depend on the Cas9 enzyme it is being paired with. The Cas9 enzyme selected will require that the sgRNA is designed specifically for use with that enzyme and will control the design of the sgRNA.

The second portion of the sgRNA which is at the 3′ end of the sgRNA is the scaffold that interacts with the Cas protein and is specific for each Cas protein.

The combinatorial methods described herein allow for the generation of novel guide nucleic acids, including novel scaffold sequences, and identification of candidate guide nucleic acids based on having a desired property. Suitably the desired property may be selected for binding affinity to the desired Cas protein, cleavage activity, or any other suitable property. Suitably, the combinatorial methods described herein may allow for generation of novel sgRNAs that have both high binding affinity for a Cas protein and high cleavage activity while maintaining high specificity.

### Guide RNA Compositions:

One aspect of the present invention provides a composition comprising a gRNA scaffold comprising a sequence selected from SEQ ID NOs: 1-134 or a gRNA sequence having at least 95%, 96%, 97%, 98%, or 99% identity thereto. Accordingly, in one aspect of the current disclosure, compositions comprising a gRNA scaffold comprising a sequence selected from the group consisting of SEQ ID NO: 1-134 is provided. In some embodiments, the composition further comprises a gRNA target region. In some embodiments, the gRNA target region is about 20 nucleotides in length and is proximate to the protospacer adjacent motif (PAM). In some embodiments the target region is about 19, 18, 17, 16, 15 or less nucleotides in length. In some embodiments, the target region is about 21, 22, 23, 24, 25 or more nucleotides in length. In some embodiments, the target region is complementary to a DNA strand in a coding region of a plant or animal gene or regulatory sequence. By way of example, and not limitation gene regulatory sequences include promoters, enhancers, silencers, transcription factor binding sites, insulators, untranslated regions, cis-regulatory elements, trans-acting factors, and metabolic response elements. The compositions provided herein may be generated using recombinant biology techniques or may be synthetically produced. Methods for recombinantly or synthetically producing gRNAs are known to those of skill in the art.

In some embodiments, the composition further comprises a Cas protein capable of forming a ribonucleoprotein (RNP) complex with at least one of the gRNA scaffold. In some embodiments, the Cas protein is a Staphylococcus aureus (Sa) Cas protein or a homolog or genetically engineered variant thereof. In some embodiments, the composition has Cas mediated DNA cleavage activity. DNA cleavage refers to the process of breaking the phosphodiester bonds in DNA, effectively cutting the DNA molecule into smaller fragments. Also envisioned are Cas nickases or Cas enzymes lacking enzymatic activity.

Although the Examples demonstrate the generation of sgRNA suitable for use in DNA cleavage or editing, the methods disclosed herein may be readily extended to the generation of sgRNA suitable for use in RNA cleavage or editing, such as with a CRISPR-Cas13 system (Cox, David B. Science 358(6366) 1019-1027 (2017).

In some embodiments, a construct is provided. In some embodiments, the construct optionally comprises a first polynucleotide encoding a Cas9 protein operably linked to a promoter to allow expression of the Cas9 protein and a second polynucleotide operably linked to a promoter, wherein the second polynucleotide comprises a sequence capable of encoding a gRNA scaffold selected from the group consisting of SEQ ID NO: 1-134 or a sequence having at least 95%, 96%, 97%, 98%, or 99% identity to at least one of SEQ ID NO: 1-134. In some embodiments, the construct further comprises a 5′ ITR (Inverted Terminal Repeat) and a 3′ ITR. In some embodiments an AAV vector comprising one of the constructs described herein is provided.

As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies.

A “vector” is any means by which a nucleic acid can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes”, that is, that replicate autonomously or can integrate into a chromosome of a host microorganism. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium. In some embodiments, the construct is an expression construct, a vector or a viral vector. Suitable vectors include, but are not limited to, plasmids, expression vectors, lentiviruses (lentiviral vectors), adeno-associated viral vectors (rAAV), among others. A preferred vector is an adeno-associated vector.

### Methods of Using:

Another aspect of the present disclosure provides a method of using a composition provided herein, a construct provided herein or vector provided herein for gene editing in a cell comprising introducing the composition, construct, or vector into a cell and selecting the cell comprising an edited target gene. The introduction of the compositions, constructs or vector into the cell will allow for genetic editing of the cell to produce an edited cell. The edited cels may then be selected using procedures known in the art.

Methods of Generating the Evolved gRNAs:

Another aspect of the present disclosure provides a method of generating a guide nucleic acid (gRNA) capable of binding a Cas protein, the method comprising a) generating a RNP complex pool by combining a Cas protein with a gRNA having a conserved target region and a randomized scaffold region; b) introducing a target DNA bound to or capable of binding to a an affinity reagent, wherein the affinity reagent may be bound to or capable of binding a bead or surface to capture the RNP, wherein the DNA comprises a PAM site and sequence complementary to the conserved target region of the gRNA; c) mixing the RNP complex with the affinity reagent and target DNA to generate an RNP-DNA-affinity reagent mixture; d) separating the RNP-DNA-affinity reagent complex from the mixture, and; e) harvesting the gRNA from the RNP-DNA-affinity reagent complex.

As used herein, a ribonucleoprotein (RNP) complex comprises a Cas protein and a gRNA. In some embodiments, the gRNAs are labeled, for example radiolabeled, with ³²P or with a fluorophore or other reagent. In some embodiments, the Cas protein is a SaCas9 protein. In some embodiments, the affinity reagent is streptavidin or biotin, but other affinity reagents such as antibodies and their binding partners may be used. The target DNA is labeled such that binding of the target DNA to the RNP can be detected and the RNP capable of binding the target DNA can be harvested. One means of doing this is the use of beads coated with a reagent that binds the affinity reagent. In some embodiments the beads are magnetic beads or fluorescent beads to allow sorting or collection of the beads. The affinity reagent may be prebound to the means of harvesting or separating the RNP-DNA complex. In one embodiment, the DNA is labeled with an affinity reagents such as biotin and bound to a streptavidin surface or bead prior to introduction and incubation with the RNP complex. The buffer in which the RNP complex and DAN are mixed may be varied to select for higher affinity or avidity or lower affinity and avidity interactions between the RNP and the target DNA. For example, the amount of Tween 20 in the buffer was varied and shown to alter the interaction and selection steps.

In some embodiments, the gRNA is capable of directing the Cas protein to perform site-specific cleavage of a targeted double-stranded DNA proximate to a PAM site. In some embodiments, the method further comprises sequencing the gRNA. In some embodiments, the method further comprises reverse transcribing and cloning the gRNA into a vector. In some embodiments, the method further comprises reverse transcribing and amplifying the gRNA. In some embodiments, the method further comprises repeating steps a) through e) of the method multiple times or rounds. Repeated rounds of this process can be used to select those gRNAs best capable of both binding to the Cas9 and target DNA and effecting cleavage of the DNA target by the Cas protein.

In some embodiments, the target DNA is labeled on the 5′ end proximal to the PAM site. See FIG. 1B. In some embodiments, the target DNA is labeled on the 5′ end distal to the PAM site. In some embodiments, the target DNA and the beads are each labeled with one of biotin or streptavidin, such that the target DNA binds to the bead via a streptavidin-biotin interaction. The bead can be used to isolate the target DNA and any RNP complex bound to the target DNA.

Suitably, the candidate guide nucleic acids may be comprised of naturally occurring, non-naturally occurring, or any combination of naturally occurring and non-naturally occurring ribo- and deoxyribonucleotides. Suitably, the non-naturally occurring nucleotides may have nucleotides with base modifications (e.g., 2-thiouridine, N6-methyladenosine, or pseudouridine), backbone modifications (e.g., phophorothioate or boranophosphate), sugar modifications (e.g., 2′-OMe, 2′-F, LNA, 2′-NH₂), 5′ and/or 3′ covalent linkages to a variety of molecular entities, or any combination thereof. The molecular entities covalently linked to the 5′ and/or 3′ end may include detection tags (e.g., biotin), labels (e.g., fluorescent dyes), proteins, lipids (e.g., cholesterol or derivatives thereof), PEG, or any combination thereof. Guide nucleic acids with base modifications may result in guide nucleic acids having increased nuclease resistance, increased complex stability, improved gene editing function, allow for in vivo expression or delivery, provide novel molecular interactions, or any combination thereof depending on the modifications selected.

In some embodiments, the guide nucleic acids generated by the methods disclosed herein may have a functional site. As used herein, a functional site has a function independent of the guide nucleic acids' ability to bind a Cas protein and guide the Cas protein to a target nucleic acid. The guide sequences generated by the methods disclosed herein that possess full functionality may be used to rationally identify, design, or construct guides that have these functional sites built into them while still maintaining the structure and functionality of the guide. In some embodiments, the guide nucleic acids may be generated by the use of a template-conserved functional site, such as a template-conserved miRNA binding domain or a template-conserved miRNA domain.

In some embodiments, the functional site may be a miRNA or other regulatory domain. Such a guide nucleic acid may have a use in regulation of cellular functions via RNA silencing and post-transcriptional gene expression.

In some embodiments, the functional site may be a miRNA binding or other binding domain. Such a guide nucleic acid may allow for competitive inhibition in a particular environment. In other embodiments, the guide nucleic acid is selected such that it doesn't have a miRNA binding or other binding domain. Identification of active guides that do not have complementarity to miRNAs or other compounds capable of binding the guide in particular cells to create more active editors. This approach would enable the regulation of Cas cleavage profiles within a given cell type and/or temporarily alter cellular functions by giving the guide nucleic acid Cas-independent siRNA like functions without significantly altering the cleavage activity of the Cas9 ribonucleoprotein complex itself. Significant differences may exist in cleavage activity depending on the target cell type in comparison to the wild type gRNA sequence. Some guides generated with the methods described herein have very little cleavage activity in one cell type while displaying cleavage activity on par with the template in others. In some embodiments, this difference may be due to the alteration of micro-RNA binding sites within guides interfering with micro-RNAs of the cell. Use of a binding domain allows for cell or tissue specific activity. For example, miRNA-122 is one of the few micro RNAs highly specific for liver expression and it is one of the highest expressed micro-RNAs in the human body. Roughly 60-70% of micro RNAs in the liver consist of miRNA-122. The guide nucleic acid may be designed to have a site complementary to miRNA-122. The purpose of this is to inhibit guides in a tissue specific fashion utilizing the micro-RNAs that are highly expressed and tissue specific. A complementary sequence in high abundance will be sufficient to inhibit the guide nucleic acids function. This allows for Cas regulation systems revolving around cell and tissue specific expression to be built that either supplement or antagonize endogenous micro-RNA activity.

In some embodiments, the functional site may be a label for detecting or monitoring activity. For example, a guide may be designed to contain sequences targeted to GFP. Guides that contain a siRNA sequence targeted towards GFP should be able to knock down the expression of GFP via sequestration and degradation of the GFP mRNA transcript. This will allow for assaying functionality.

Prime Editing gRNAs

Prime editing represents a gene editing approach that may install specific DNA sequence substitutions, insertions, and deletions without requiring double-stranded breaks or donor DNA. The system utilizes a Cas9 nickase (nCas9), a reverse transcriptase, and an extended prime editing guide RNA that includes additional material on the 3′ end to be reverse transcribed directly into the genomic target during prime editing. The selection approaches provided herein address challenges associated with scaffold sequences that can elicit deleterious self-folding interactions when paired with different sets of genomic targets in a sequence-dependent manner. In some cases, variant sequences outperform canonical sequences at subsets of genomic targets and can rescue functionality at sites that are traditionally difficult to edit using CRISPR-Cas9 systems

In brief, the inventors developed a novel approach using SELEX to evolve and identify functional evolved pegRNA scaffolds and a method to select the edited sequences after prime editing in vitro. This new high throughput selection method can create scaffold sequences which are enriched for their ability to encode genomic edits rather than merely cleave DNA. Our proof-of-concept studies indicate that pegRNA scaffolds can be evolved for increased activity at any genomic target with any intended substitution, insertion, or deletion. To demonstrate viability of the new method, we will evolve therapeutically relevant pegRNAs with the goal of rescuing editing functionality at genomic sites where prime editing is not efficient using current technologies.

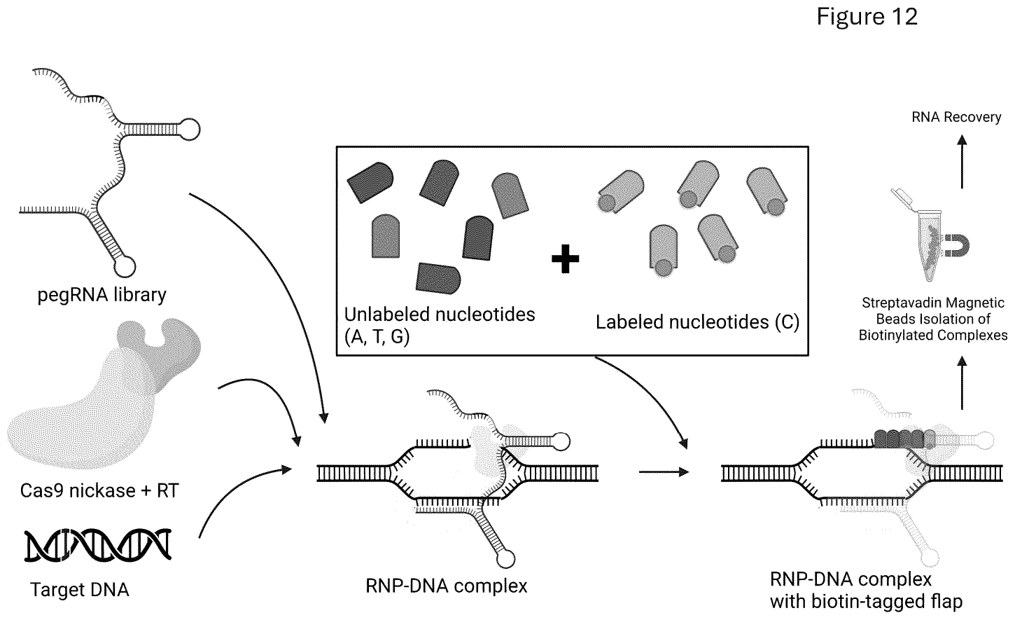

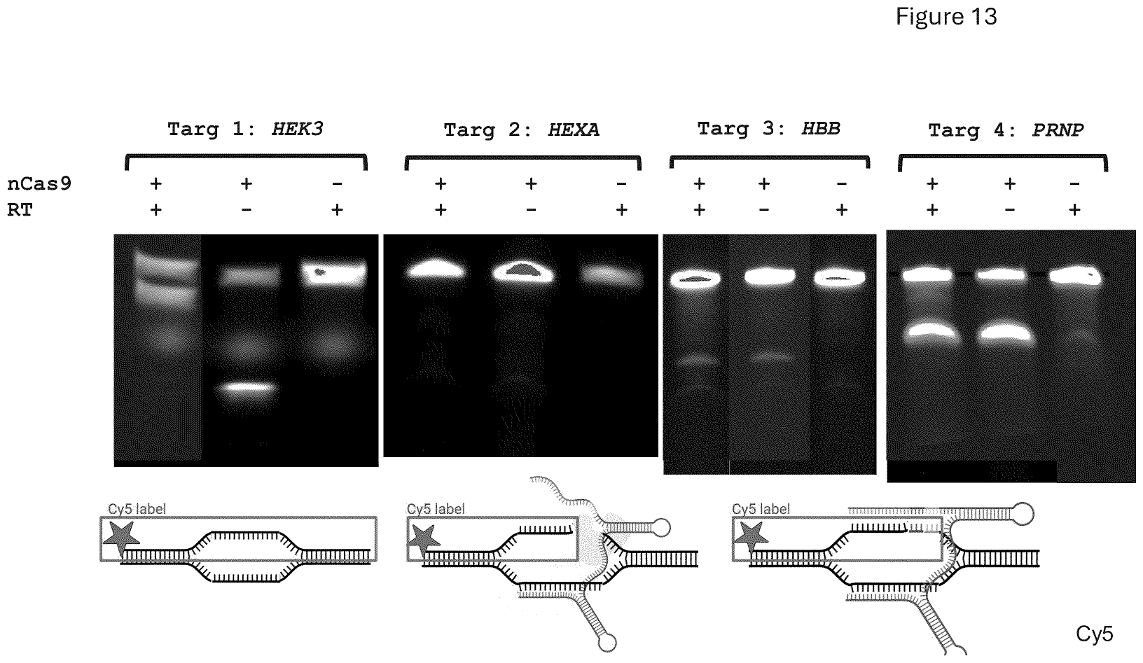

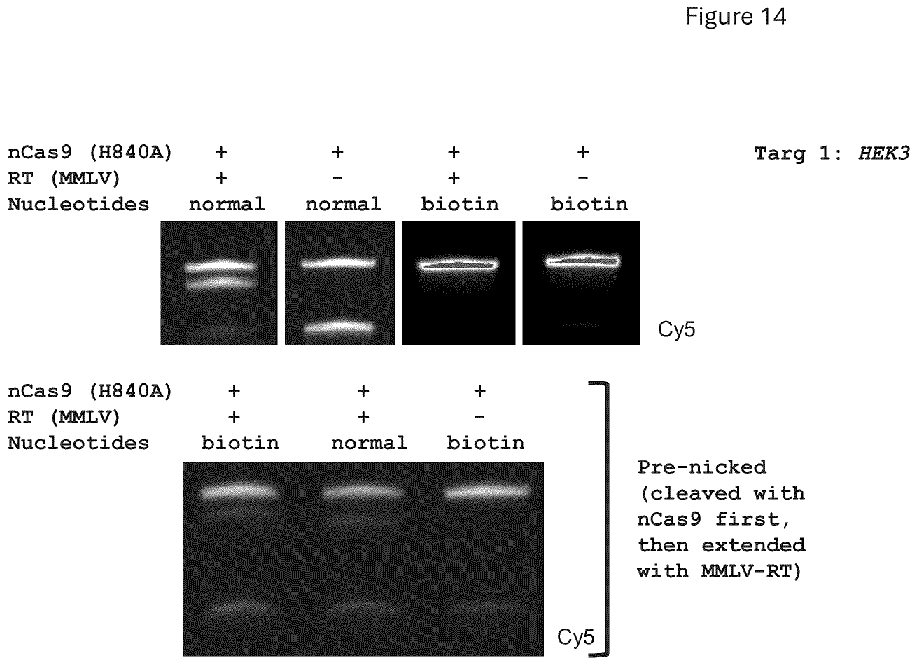

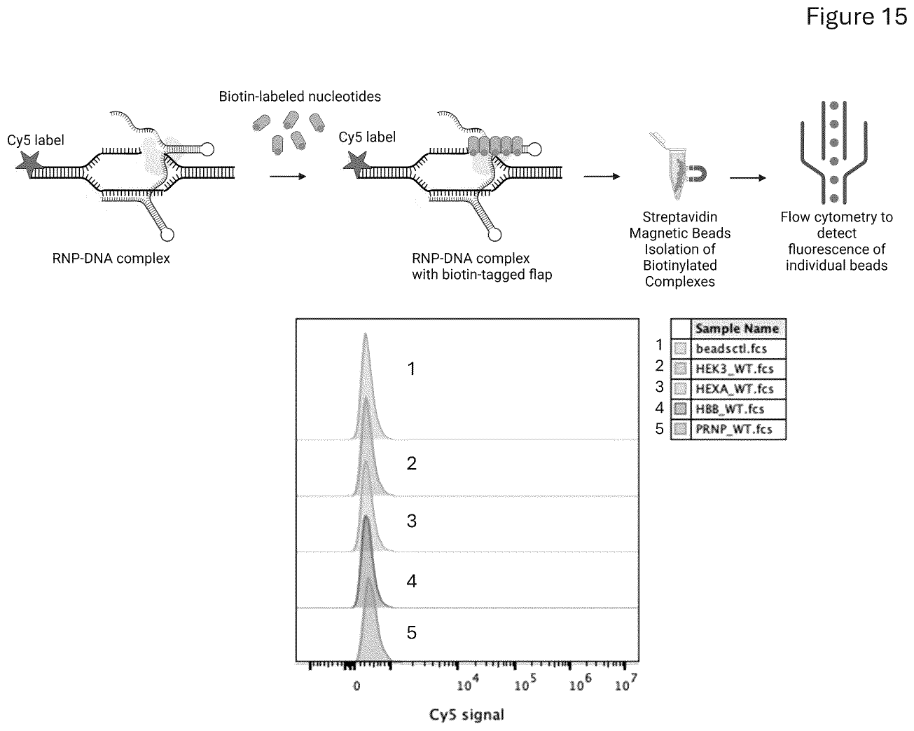

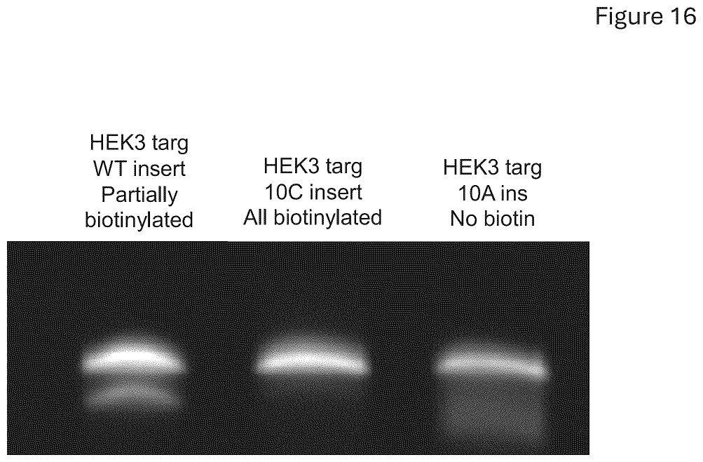

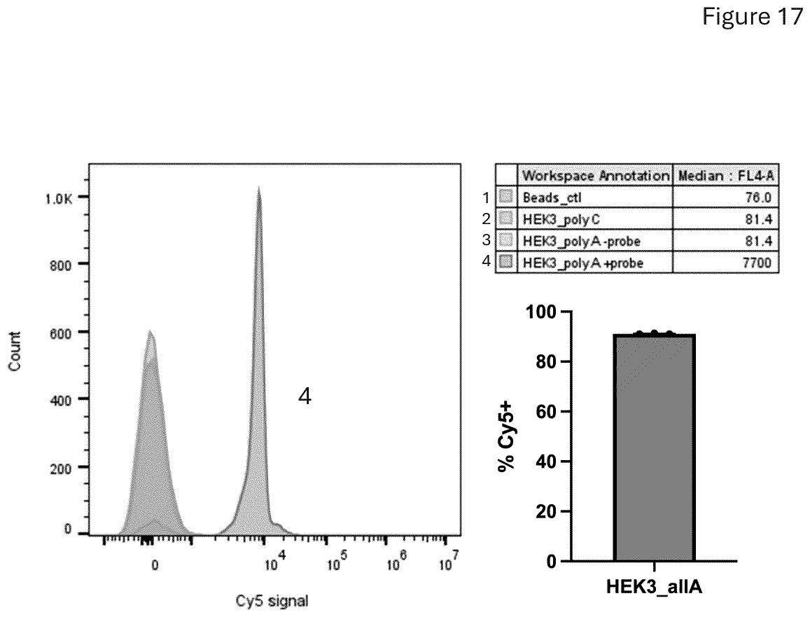

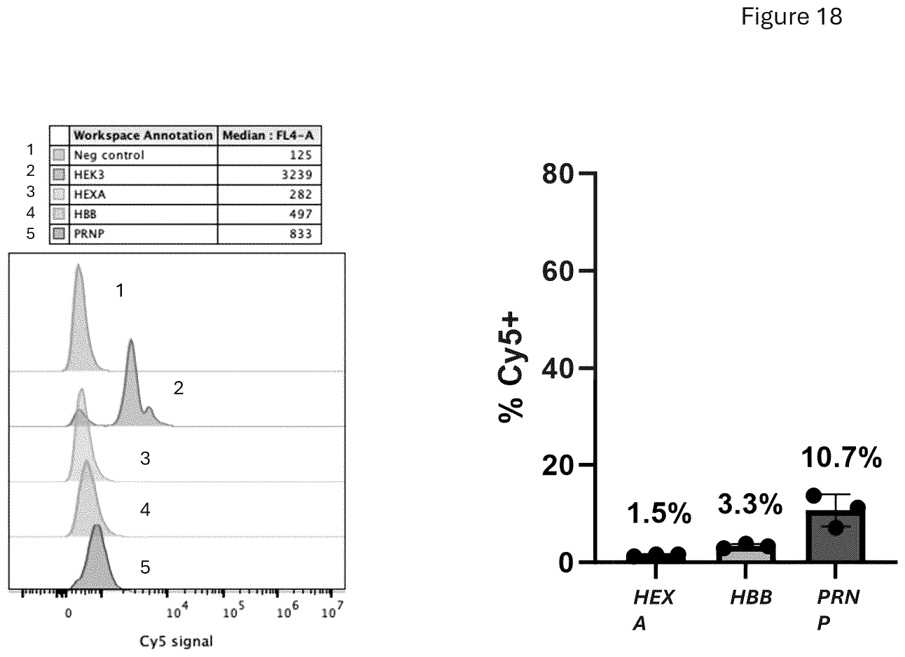

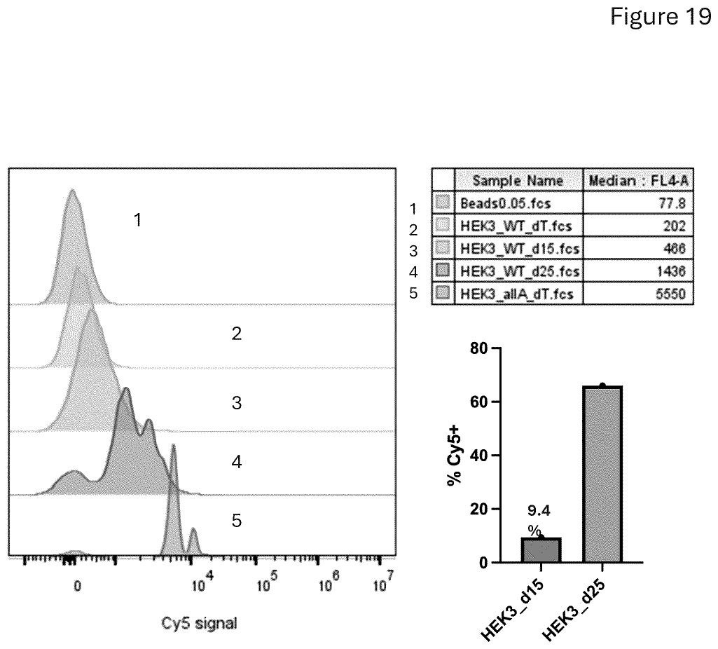

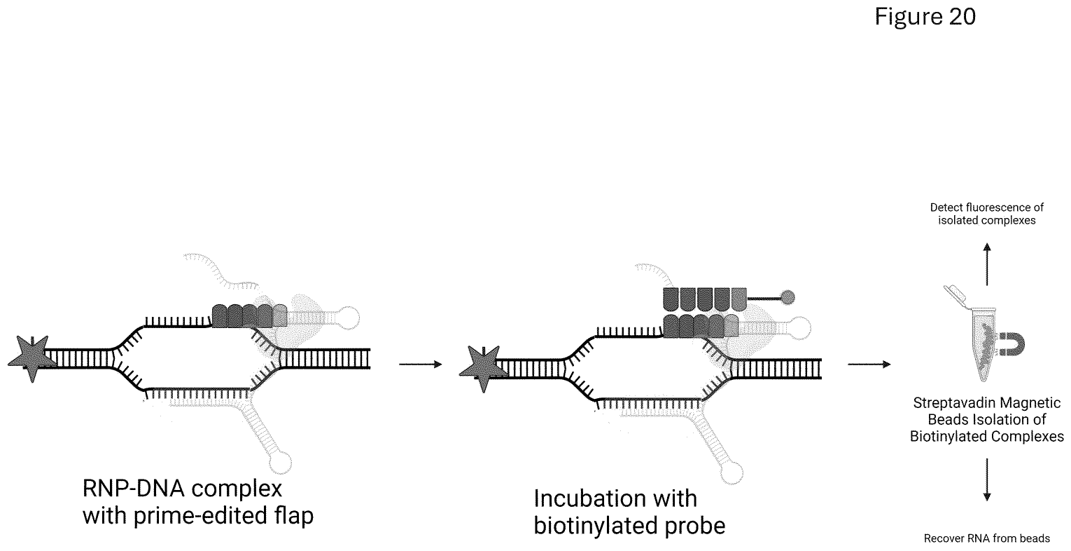

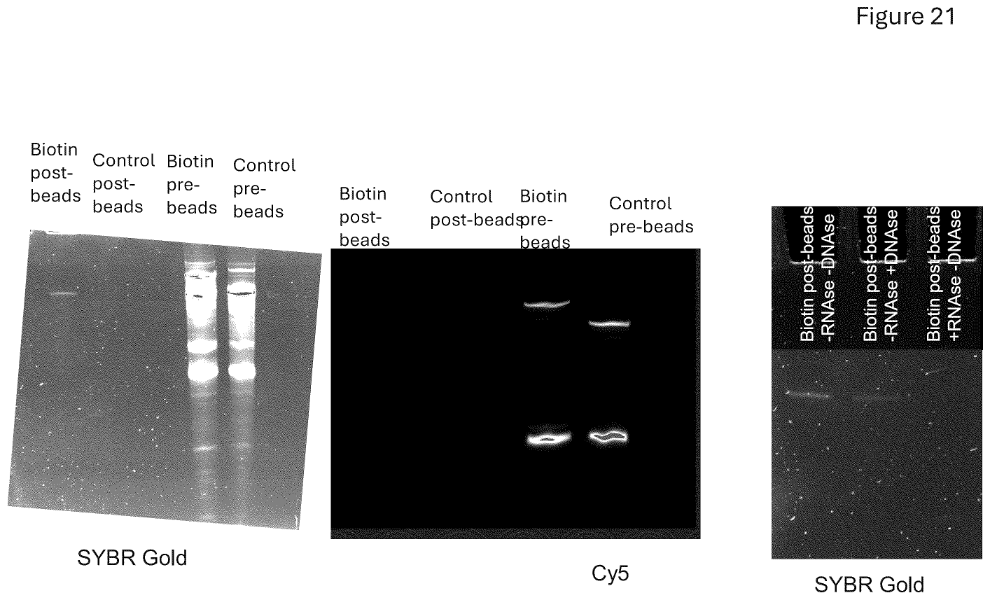

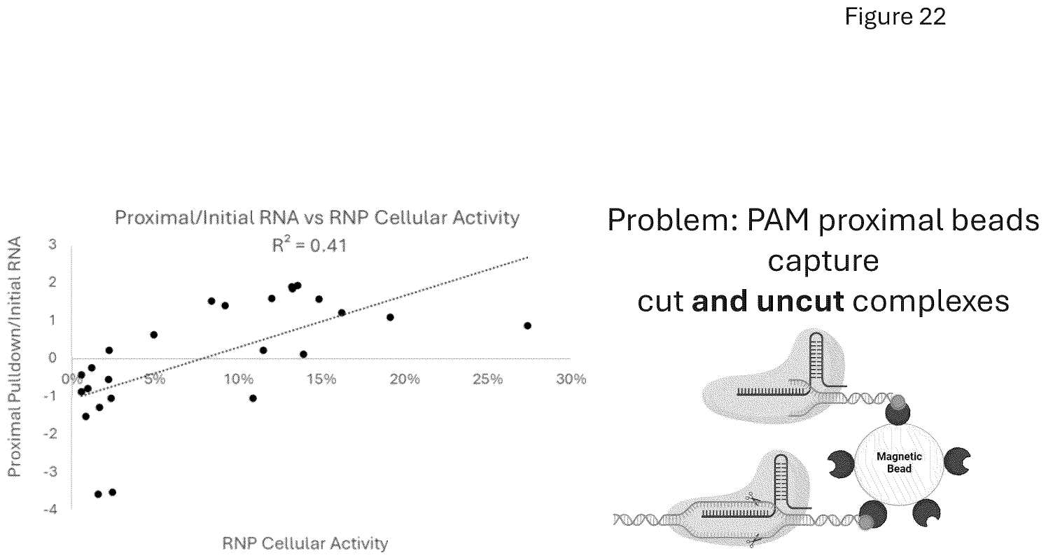

As shown in FIG. 12, the method begins by generating a RNP-DNA complex. In some embodiments, the RNP-DNA complex is generated using Prime editing. Prime editing is a CRISPR-Cas9 system which can install specific DNA sequence substitutions, insertions, and deletions without requiring double-stranded breaks or donor DNA using a Cas9 nickase, a reverse transcriptase, and an extended “prime editing guide RNA” (pegRNA). pegRNAs include additional material on their 3′ ends to be reverse transcribed directly into the genomic target during prime editing. The DNA flap of the RNP-DNA complex is biotinylated or labeled with a biotinylated probe. Next, the biotinylated complexes are separated using a mechanism such as streptavidin coated magnetic beads.

The selection process may involve generating ribonucleoprotein-DNA complexes followed by labeling the edited DNA e.g., via biotinylation, and isolation steps to enrich for functional variants. Proof-of-concept studies some of which are provided in the examples indicate that pegRNA scaffolds can be evolved for increased activity at genomic targets with intended substitutions, insertions, or deletions. The methods may be applied to evolve therapeutically relevant pegRNAs with the goal of rescuing editing functionality at genomic sites where prime editing efficiency may be limited using existing technologies. Variant scaffolds with sufficient sequence diversity from canonical scaffolds may ameliorate deleterious self-folding interactions at traditionally low-efficiency spacers and targets, thereby rescuing function and making the genome more tractable to editing.

The prime editing guide RNA (pegRNA) may serve as a central component that directs the editing process through its structural organization. The pegRNA comprises a 5′ region and a 3′ region that are complementary to the target DNA, allowing for specific binding and recognition of the genomic target site. The pegRNA further includes an intended edited region that may be incorporated into the target DNA through the reverse transcription process. This intended edited region contains the template information for the desired genomic modification, whether the modification involves nucleotide substitutions, insertions, or deletions. The structural organization of the pegRNA allows the molecule to function both as a guide for target recognition and as a template for the editing process.

The prime editing protein complex comprises a Cas9 nickase and a reverse transcriptase (RT) that work in coordination to execute the editing process. The Cas9 nickase component may generate a single-strand nick in the target DNA at the specified location, creating the entry point for the editing process. The reverse transcriptase component may then utilize the pegRNA template to synthesize the complementary DNA sequence that incorporates the intended edit. This dual-protein system allows for precise control over both the targeting and the template-directed synthesis phases of the editing process. The integration of these two enzymatic activities within a single protein complex may facilitate efficient coordination between the nicking and reverse transcription steps.

Target DNA may comprise any genomic sequence that contains the recognition site for the pegRNA and represents the substrate for the editing process. The target DNA provides the template strand that may be nicked by the Cas9 nickase component, creating the substrate for reverse transcription. The complementary regions of the pegRNA may hybridize to specific sequences within the target DNA, establishing the precise location where the editing process will occur. The target DNA sequence context may influence the efficiency of the prime editing process, with some genomic locations being more amenable to editing than others. The interaction between the pegRNA and target DNA is sequence-dependent and the length of the complementarity between the pegRNA and the target DNA is dictated by the Cas 9 requirements.

Another aspect of the present disclosure provides a method of generating a pool of prime editing gRNAs (pegRNA). Prime editing enables precise small insertions, deletions, and base swaps, offering unparalleled precision. Unlike traditional CRISPR systems, prime editing executes these changes without introducing double-stranded DNA breaks, and targeted insertions are achieved without requiring donor DNA templates. Prime editing uses a Cas9 nickase, a variant that nicks only one strand of the DNA. This Cas9 nickase is fused to a reverse transcriptase enzyme, forming what is known as a prime editor (PE). Reverse transcriptase, an enzyme commonly found in retroviruses. Within the context of prime editing, reverse transcriptase uses the RNA sequence provided by the (pegRNA) to generate and insert the desired DNA changes directly into the genome.

A pegRNA consists of four primary sequence parts: the target sequence, typically about 20 nucleotides, directs the Cas9 nickase to the specific DNA site for editing; the scaffold sequence that forms a secondary structure that binds Cas9 nickase, enabling it to function, which is typically about 76 nucleotides long, but may be 70-80 nucleotides in length; the reverse transcription template sequence contains the desired edit and homology sequence. The typical size is approximately from about 10-40 nucleotides depending on the length of the desired genetic alteration; and the primer-binding site (PBS), which is about 10-15 nucleotides in length, serves as an anchor point for the reverse transcriptase to initiate DNA synthesis. The total length of a pegRNA generally falls between 120 and 145 nucleotides and can extend to about 225 nucleotides long. Due to the specific uses and structural necessity of each region of the pegRNA, it is not obvious how much of the pegRNA can be modified. As described herein, the inventors provide a novel means for generating and selecting pegRNA.

The length of the pegRNA, and in particular, the reverse transcription template sequence can negatively influence pegRNA folding and activity. Thus, as described herein, the inventors, have developed a novel approach using SELEX to evolve and identify functional pegRNA scaffolds. This new high throughput selection method can create scaffold sequences which are enriched for their ability to encode genomic edits rather than merely cleave DNA. The inventors demonstrate that pegRNA scaffolds can be evolved for increased activity at any genomic target with any intended substitution, insertion or deletion.

In some cases, the pegRNA may be provided as a pool of pegRNA comprising at least 10, at least 20, at least 30, at least 40, at least 50, at least 60, at least 70, at least 80, at least 90 or at least 100 pegRNAs designed to edit the same target DNA and including the intended edited region. This pool configuration allows for the simultaneous evaluation of multiple pegRNA variants that target the same genomic location but may differ in their scaffold sequences or other structural features. The pool approach may facilitate the identification of pegRNA variants with enhanced editing efficiency or improved performance characteristics or simply allow selection of the desired edited DNA more quickly and efficiently than without the pool approach or the selection approach provided here. Each pegRNA within the pool may contain the same target binding sequence for target recognition and the same intended edit template, while varying in other structural elements that could affect functionality. The use of pegRNA pools may enable comparative analysis of different scaffold configurations and their impact on editing efficiency.

The generation of ribonucleoprotein-DNA complexes represents a foundational step in the prime editing process that brings together the pegRNA, prime editing protein complex, and target DNA into a functional assembly. The ribonucleoprotein-DNA complex forms through the specific binding interactions between the pegRNA and the Cas9 nickase component of the prime editing protein complex, combined with the hybridization of the pegRNA spacer sequence to the complementary region within the target DNA. The formation of this complex may be facilitated by the presence of appropriate buffer conditions, ionic strength, and temperature parameters that promote stable protein-RNA and RNA-DNA interactions.

The incubation process allows the assembled ribonucleoprotein-DNA complex to undergo the sequential enzymatic reactions that constitute the prime editing mechanism. During incubation, the Cas9 nickase component may generate a single-strand nick in the target DNA at the position specified by the pegRNA spacer sequence and the protospacer adjacent motif recognition. The nicking reaction creates a free 3′-hydroxyl group on the target DNA strand that serves as the primer for the subsequent reverse transcription reaction. The reverse transcriptase component may then utilize this 3′-hydroxyl group to initiate DNA synthesis using the pegRNA template region as the guide for incorporating the complement of the intended edit. The incubation conditions, including temperature, buffer composition, and reaction time, may influence the efficiency of both the nicking and reverse transcription steps.

The reverse transcriptase incorporation process synthesizes new DNA sequence that contains the complement of the intended edit into the nicked strand of the target DNA. This synthesis reaction extends from the nick site and incorporates nucleotides according to the template sequence provided by the pegRNA, effectively copying the intended edit information into the genomic DNA. The newly synthesized DNA strand creates a heteroduplex structure where one strand contains the original target sequence and the other strand contains the edited sequence. The length of the reverse transcribed region may depend on the template sequence provided by the pegRNA and the processivity of the reverse transcriptase enzyme. The incorporation process generates a single-strand DNA incorporated edit that represents the initial product of the prime editing reaction before any subsequent cellular DNA repair processes occur.

The nucleic acid probe sequence may be designed to be complementary to the single-strand DNA incorporated edit that results from the prime editing process. The probe sequence may hybridize specifically to the edited DNA strand through Watson-Crick base pairing interactions, allowing for selective recognition and binding of the edited sequences. The complementary nature of the probe sequence may enable discrimination between edited and unedited DNA strands, facilitating the enrichment process for successfully edited targets. The probe design may take into account the specific sequence of the intended edit to ensure optimal binding affinity and specificity. The length and composition of the complementary region may influence the stability of the probe-target interaction and the overall efficiency of the selection process.

The probe may further comprise a homopolynucleotide sequence that extends beyond the complementary region and provides additional functionality for the selection process. Homopolynucleotide sequences may consist of repeating units of a single nucleotide type, creating regions of uniform composition that can serve specialized functions in the probe design. The length of the homopolynucleotide sequence may vary depending on the intended application and the binding requirements for subsequent selection steps. The incorporation of homopolynucleotide sequences may provide sites for specific binding interactions with complementary homopolynucleotide probes or other molecular recognition elements. The homopolynucleotide may be a polyadenine or polycytosine sequence that provides specific binding characteristics for the selection process. Homopolynucleotide sequences may consist of multiple consecutive nucleotides that can form specific interactions with complementary sequences. These sequences may range in length from short stretches of a few nucleotides to longer sequences that provide enhanced binding capacity and specificity.

The homopolynucleotide may be added using Terminal deoxynucleotidyl transferase (TdT), an enzyme that can add nucleotides to the 3′ end of DNA molecules without requiring a template strand. TdT may be used to append homopolynucleotide tails to existing probe sequences, allowing for the post-synthetic modification of probe molecules to incorporate the desired homopolynucleotide functionality. The TdT reaction may be controlled to add specific numbers of nucleotides by adjusting reaction conditions, enzyme concentration, and incubation time. For example, the number of nucleotides added during the TdT reaction is controlled by the reaction incubation time, which can be extended for any amount of time depending on how many nucleotides are to be added. By way of example, and not limitation, methods described herein utilize a reaction time of approximately about 20 minutes to add the homopolynucleotide tail. The enzyme may preferentially add specific nucleotide types when provided with single nucleotide substrates, enabling the creation of polyadenine, polycytosine, or other homopolynucleotide sequences. The TdT-mediated addition process may provide a flexible approach for modifying probe sequences after their initial synthesis, allowing for the customization of probe properties for specific applications. The homopolynucleotide portion of the probe may be capable of binding a complementary homopolynucleotide comprising a label, creating a two-step labeling strategy where the probe first binds to the edited DNA and then associates with a labeled complementary sequence. The complementary homopolynucleotide may contain biotin, fluorescent dyes, or other detectable labels that enable the identification and isolation of probe-bound complexes.

Prime edited polyadenine inserts may be pulled down via biotin-oligo(dT) probe instead of direct biotinylation of the complex, providing an alternative labeling strategy that targets the edited sequence rather than the protein components. The biotin-oligo(dT) probe may consist of a polythymidine sequence conjugated to biotin molecules, creating a probe that can specifically bind to polyadenine sequences through complementary base pairing. This approach may avoid potential interference with protein function that could result from direct biotinylation of the ribonucleoprotein complex. The oligo(dT) probe may be designed with varying lengths of polythymidine sequence to optimize binding affinity and specificity for the polyadenine inserts. The biotin labeling of the oligo(dT) probe may enable subsequent isolation using streptavidin-based capture systems, providing a robust method for enriching edited sequences.

Custom-made complementary labeled-oligo probes, such as biotinylated probes, may be used to pull down wild-type prime edited inserts that do not contain homopolynucleotide modifications. These probes may be designed with sequences that are perfectly complementary to the specific edited sequences generated by the prime editing process. The labeling of these custom probes may enable direct capture and isolation of the edited DNA sequences. For example a biotin labeled probe can be separated through streptavidin-based separation methods. The design of custom complementary probes may require knowledge of the specific sequence changes introduced by the prime editing process to ensure optimal binding specificity.

In some embodiments, the probe comprises at least one locked nucleic acid. A locked nucleic acid (LNA) is a synthetic nucleic acid analogue with a bicyclic sugar unit that increases its stability and binding affinity to complementary DNA or RNA sequences. This “locking” involves a methylene bridge between the 2′-oxygen and 4′-carbon of the ribose ring, restricting the ring's flexibility. LNAs can be incorporated into polynucleotides such that all of the nucleotides are LNA, or the polynucleotide can contain a mixture of traditional nucleotides and LNA. Generally, a probe may comprise 6 or fewer LNA, with 4 or fewer being consecutive, with the mismatched base and both adjacent bases being a LNA, and wherein the two furthest bases at the 5′ end and the furthest base at the 3′ end of the probe are not LNAs. For example, SEQ ID NO: 349 contains 5 LNA in a polynucleotide of 14 nucleotides in length. Polynucleotides provided herein may comprise, one or more LNA such that only one of the nucleotides in the polynucleotides is an LNA or all of the nucleotides are LNA, and any amount in-between.

Probes of the present invention can be of any length necessary to allow for detection and separation. In some embodiments, use of particular modifications alters the length of the probe. For example, the use of LNA may allow for a shorter probe. Longer probes may provide increased binding affinity through multiple base pairing interactions without the need of use of LNAs, potentially resulting in more stable probe-target associations. The experimental data indicates that 25 base pair probes significantly outperformed 15 base pair probes in terms of capture efficiency, even when the prime edited insert may be only 10 base pairs in length. This enhanced performance of longer probes may result from increased binding stability, reduced dissociation rates, or improved accessibility of the biotin label for subsequent capture steps. The optimal probe length may depend on factors such as the target sequence composition, the reaction conditions, and the specific requirements of the selection process. The probe length range may be determined through empirical testing to identify the optimal balance.

In some embodiments, the pegRNA is capable of binding to a Cas protein or a modified Cas protein. As used herein, a pegRNA that is capable of binding to a Cas protein may be bound to a Cas protein, or a pegRNA that if contacted by a Cas protein, the pegRNA would bind to the Cas protein. Cas proteins are endonucleases that use a single guide RNA (sgRNA) to form complementary base pairs with target DNA and then cleave the DNA at specific sites. Any Cas protein described herein may be used. A modified Cas protein may comprise modifications to alter specificity, reduce off-target effects, alter stability, or appending functional structures to alter function. In some embodiments, the pegRNA is bound to Cas9. In some embodiments, the Cas protein is a nickase. A Cas nickase is a modified CRISPR-Cas enzyme that is engineered to cut only a single strand of DNA, rather than both strands like a standard Cas nuclease. By inactivating one of its two nuclease domains, it creates a single-stranded nick. In some embodiments, the modified Cas9 protein comprises a Cas9 fusion protein. In some embodiments, the Cas9 fusion protein comprises a Cas9 reverse transcriptase fusion protein. In Prime Editing, the reverse transcriptase, fused to the Cas9, reads the template section of the pegRNA and synthesizes a new DNA strand that incorporates the desired edit.

The Cas protein described herein can be of any origin. In some embodiments, the Cas protein, or modified Cas protein is a Streptococcus pyogenes or Staphylococcus aureus Cas9 protein. Staphylococcus aureus Cas9 (SaCas9) produces blunt=ended double stranded breaks and autonomously releases the DNA targets' PAM-distal end after cleavage, allowing it to function as a multiple-turnover enzyme. In contrast Streptococcus pyogenes Cas9 (SpCas9) produces staggered double stranded breaks leaving a 5′ overhand of a few nucleotides and remains bound to both ends of the cleaved DNA for an extended period, making it a single turn-over nuclease. In some embodiments, modifications to the biotinylated probe described herein may be altered depending on if spCas9, saCas9, or some other Cas9 is used. For example, using TDT enzyme to add a polyA may not be functional with a saCas9.

In some embodiments, the method described herein further comprises recovering the pegRNA from the separated edited DNA. Methods of recovering the pegRNA from the separated edited DNA are known in the art. Generally methods include chemical, enzymatic or physical (bead or silica based) extraction following by using reverse transcriptase and sequencing to identify and recover the pegRNA from a sample.

In some embodiments, the gRNA or pegRNA described herein may be used in a method of editing DNA. Editing DNA may comprise changing one or more nucleotides, adding one or more nucleotides, removing one or more nucleotides or combinations thereof. In some embodiments, editing is selected from the group consisting of at least one nucleotide is changed, an insertion is made, a deletion is made and combinations thereof.

### Kits:

The present disclosure further provides kits for carrying out the subject methods as provided herein. For example, in one embodiment, a subject kit may comprise, consist of, or consist essentially of one or more of the following: target DNA, pegRNA library, Cas9 nickase+RT, and unlabeled nucleotides. In other embodiments, a kit may further include other components. Such components may be provided individually or in combinations, and may provide in any suitable container such as a vial, a bottle, or a tube. Examples of such components include, but are not limited to, one or more additional reagents, such as one or more dilution buffers; one or more reconstitution solutions; one or more wash buffers; one or more storage buffers, one or more control reagents and the like. Components (e.g., reagents) may also be provided in a form that is usable in a particular assay, or in a form that requires addition of one or more other components before use (e.g. in concentrate or lyophilized form). Suitable buffers include, but are not limited to, phosphate buffered saline, sodium carbonate buffer, sodium bicarbonate buffer, borate buffer, Tris buffer, MOPS buffer, HEPES buffer, and combinations thereof.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Additional Definitions

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps.

All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The following examples are meant only to be illustrative and are not meant as limitations on the scope of the invention or of the appended claims.

### EXAMPLES

The following Examples are illustrative and should not be interpreted to limit the scope of the claimed subject matter.

### Example 1: BLADE Directed Evolution of SaCas9 gRNAs Improves Gene Editing Efficiency

CRISPR-based editing is inefficient at over two thirds of genetic targets. A primary cause is RNA misfolding that can occur between the spacer and scaffold regions of the gRNA, which hinders the formation of functional Cas9 ribonucleoprotein complexes (RNPs). Here, we uncover hundreds of highly efficient gRNA variant scaffolds for Staphylococcus aureus (Sa)Cas9 utilizing an innovative BLADE (Binding and Ligand Activation Driven Enrichment) methodology, which leverages asymmetric product dissociation over rounds of evolution. SaBLADE-derived gRNA scaffolds contain 7-42% of nucleotide variation relative to wildtype. gRNA variants are able to improve gene editing efficiency at all targets tested, and they achieve their highest levels of editing improvement (>400%) at the most challenging DNA target sites for the wildtype SaCas9 gRNA. This arsenal of SaBLADE-derived gRNA variants showcases the power and flexibility of combinatorial chemistry and directed evolution to enable efficient gene editing at challenging, or previously intractable, genomic sites.

CRISPR-Cas9 nucleases possess target programmability through their guide (g)RNAs 1-4. Target sites are determined through base pairing between DNA and the first ˜20 nucleotides of the gRNA, termed the “spacer”. The remaining 80 nucleotides of the gRNA, known as the scaffold, fold into three stem loops which bind and activate the Cas9 protein to form a functional ribonucleoprotein complex (RNP) with endonuclease activity^{1,5}. Unfortunately, a large proportion of gRNA spacers mediate inefficient gene editing, a trend which holds consistent among various orthologs⁶⁻⁸. Widely adopted solutions have focused on predicting rather than rescuing the DNA cleaving activities of gRNAs⁸⁻¹¹, yet precision editing modalities used in the generation of animal models, cell lines and therapeutics require effective targeting at specific genetic locations^{2,12,13}, limiting progress in precision editing projects using low efficiency CRISPR-Cas9.

A major cause of low editing efficiency is the misfolding of the spacer and scaffold regions of the gRNA¹⁴⁻¹⁷. Activity rescue has approached this challenge through the stabilization of the gRNA secondary structure by incorporating chemical modification into the gRNA 15-18. Unfortunately, chemically modified gRNAs cannot be delivered using plasmids or gene therapy vectors, will not rescue all misfolded gRNAs and remain prohibitively expensive due to low synthesis efficiency for RNAs beyond 60 nucleotides¹⁹⁻²¹. Altering the gRNA sequence using natural nucleotides can overcome these limitations^{14,18} We previously demonstrated that a molecular evolution process based on SELEX (Systematic Evolution of Ligands by Directed Evolution)^{22,23} can be used to generate variant gRNA scaffolds for Streptococcus Pyogenes Cas9 (SpCas9) gRNA with improved fluorescent knockout of GFP positive HEK293 cells^{18,24} Herein, we optimize the Staphylococcus Aureus (SaCas9) gRNA through a new instance of BLADE (Binding and Ligand Activation Driven Enrichment) termed SaBLADE, which evolves RNA guided editors utilizing asymmetric post cleaving target dissociation kinetics^{5,25}. SaCas9 is an ortholog which due to its smaller size can be more efficiently packaged into AAV vectors, increasing its translational potential^{5,26,27}. SaBLADE yields hundreds of scaffolds variants capable of improving editing efficiency by >400% over the wildtype scaffold at difficult to edit genomic targets and highlights the power and flexibility of directed evolution methods for accelerating the development of gene editors.

### Materials and Methods

gRNA Library Generation

For library generation, three oligonucleotides were ordered from Integrated DNA Technologies (Coralville, IA); a forward oligonucleotide 5′GGGGATAATACGACTCACTATAGGCGAGGGCGATGCCACCTA3′ (SEQ ID NO: 135) consisting of the T7 promoter and spacer region, a reverse oligonucleotide 5′AAAATCTCGCCAACAAGTTGACG 3′ (SEQ ID NO: 136) consisting of the the last SaCas9 loop, and a 99 base oligonucleotide 5′AAAATCTCGCCAACAAGTTGACGC(N1)(N2)(N3)(N2)(N3)(N2)(N2)(N4)(N2)(N1)(N1)(N4)(N1)(N1)(N3)(N4)(N2)(N3)(N3)(N3)(N4)(N4)(N2)(N1)(N3)(N3)(N3)(N2)(N1)(N1)(N3)(N2)(N4)(N1)(N2)(N4)(N1)(N1)(N2)(N3)(N4)(N1)(N2)(N3)(N2)(N4)(N3)(N2)(N2)(N2)(N4)TAGG TGGCATCGCCCTCGC3′ (SEQ ID NO: 137) where semirandom base mixes are as follows (N1=14% A,14% C,58% G,14% T), (N2=58% A,14% C,14% G,14% T), (N3=14% A,14% C,14% G,58% T), (N4:14% A,58% C,14% G,14% T), corresponding to a reverse complement to the spacer region, a 60-base stretch of semirandom bases corresponding to a probability of 58% wildtype SaCas9 sequence and 14% of each other base at each position, and a fixed second stem loop. Exo-deficient Klenow extension generated dsDNA from a 100 ug (˜3 nmol) of the 99 base oligonucleotide added to 25 ug (1.9 nmol) of the forward oligonucleotide and allowed to anneal in 10 nM Tris-HCl, Ph 8.0 with 10 mm MgCl2 at 90 degrees on a heating block followed by cooling to room temperature. The double-stranded library was extracted using phenol chloroform and ethanol precipitated. Transcription was carried out using Invitrogen T7 polymerase (18033019), on a total of 0.75 nmol DNA for 4 hours at 37 degrees Celsius and digested with DNAse I (NEB). Samples were run on a precast 12% denaturing polyacrylamide gel (Biorad) in 1×TBE running buffer with 7M urea. The gel was UV shadowed the 100 base size band cut out, crushed and incubated overnight in buffer TE (10 mM Tris-HCl, 1 Mm EDTA, pH 8), and purified through an Amicon 3 k 15 ml column 6 times (Millipore UFC900308).

### SaCas9 Radioactive Optimization

Round 0 gRNA library and wildtype gRNA were radiolabeled as negative and positive controls as follows; CIP dephosphorylation was carried out (NEB M0525) and p32 radiolabeling was performed using T4 PNK (NEB M0201) and p32-labeled (Perkin). Free NTPs were removed with a 40,000 Dalton exclusion Tris Chromatography column (Biorad 7326224). The resulting radiolabeled gRNAs were incubated with SaCas9 protein at a 1:1 ratio to generate RNPs for 10 minutes at 37 degrees. These RNPs were tested for either the original SpCas9 selection strategy¹⁸ or optimized for the strategy detailed below.

PCR of a GFP mutant open reading frame bearing the target 5′GGCGAGGGCGATGCCACCTA 3′ (SEQ ID NO: 138) (PAM-CGGAAT) (SEQ ID NO: 139) was carried out using forward primer 5′ GTGAGCAAGGGCGAGGAGCTG 3′ (SEQ ID NO: 140) and reverse primer 5′ TACTTGTACAGCTCGTCCATGC 3′ (SEQ ID NO: 141), with either primer being 5′ biotin tagged, producing dsDNA GFP target amplicon with a biotin tag at either end. After PCR purification, amplicons were incubated with streptavidin beads (ThermoFisher 65002) for 2 hours in 5× molar excess relative to bead streptavidin sites followed by 3 washes with buffer NEB 3.1 supplemented with 0.006% Tween 20.

RNPs were added to either 3′ or 5′ target prebound beads, incubated for 30 minutes at room temperature, washed three times in NEB Buffer 3.1 supplemented with 0.006% tween 20, and radioactive reads were measured in a Tri-Carb 4810 TR scintillation counter (Perkin Elmer) for 1 minute. Bead pulldown signal to washed signal ratio was compared between RNP complexes formed using either WT gRNA or unenriched round 0 gRNA pool.

### SaCas9 Directed Evolution

Positive selection consisted of six positive rounds of evolution. Mixed selection consisted of 4 positive rounds, a negative round, and a final positive round. Both selections began with RNP formation between equimolar (0.2 nmol) library gRNAs and SaCas9 protein for 10 minutes at room temperature. For substrate generation, the DNA target was amplified from a GFP ORF containing the mutant target sequence 5′GGCGAGGGCGATGCCACCTACGGAAT3′ (SEQ ID N:: 142) amplified using forward primer 5′ GTGAGCAAGGGCGAGGAGCTG3′ (SEQ ID NO: 143) and reverse primer 5′ TACTTGTACAGCTCGTCCATGC 3′ (SEQ ID NO: 144), with either the forward primer bearing 5′ biotin (PAM Distal Negative Selection) or the reverse primer bearing 5′ biotin (PAM proximal Positive Selection). This target was pre-incubated with biotinylated beads for 1 hour and beads washed 3 times with 1 ml NEB Buffer 3.1.

0.02 nmol of bead-bound DNA targets were incubated with RNPs in a 10-fold molar deficit for 20 minutes at 37 degrees, magnetically pulled down and washed 3×. Beads were phenol chloroform extracted, keeping the aqueous phase, and nucleic acids were then ethanol precipitated. Recovered sample was reverse transcribed using MMLV RT and gRNA reverse primer 5′AAAATCTCGCCAACAAGTTGACG3′ (SEQ ID N: 145) per manufacturer protocol (Thermo Fisher 28025013) and PCR purified. PCR was carried out to amplify the DNA library using 5′GGGGATAATACGACTCACTATAGGCGAGGGCGATGCCACCTA3′ (SEQ ID NO: 146) and 5′AAAATCTCGCCAACAAGTTGACG 3′ (SEQ ID NO: 147) and PCR purified. T7 Transcription proceeded overnight at 37 degrees, using 4000 units of T7 polymerase (NEB M0251), in a 1600 ul reaction consisting of 0.5 mM final NTP, 1 ug DNA template, and 5 mM DTT. DNAse (NEB M0303) and 1 Mm CaCl2) was added and incubated for 1 hour at 37 degrees. gRNA was purified through gel extraction from a 12% denaturing polyacrylamide gel, and soaked overnight in buffer TE. gRNAs were washed of salts using an Amicon Ultra 3 kDA column (Millipore UFC800308) 6 times at 12000×G using ultrapure water. gRNA concentration was measured using nanodrop 2000.

Individual gRNA Transcription and Plasmid Generation

DNA templates for gRNAs preceded by a T7 promoter were produced as with library generation, using a middle primer containing a single predetermined sequence per reaction. A T7 transcription reaction to produce the gRNA was carried out as described above, scaled down to 100 units of T7 polymerase in a 40 ul reaction and purified using a 0.5 ml Amicon Ultra 3 kDA spin column.

For plasmids, gRNA DNA templates were cloned into U6 based expression plasmid PX441, using the forward primer 5′GACCACGGTCTCACACCGCCGGTGGTGCAGATGAACTT3′ (SEQ ID NO: 148) containing a BsaI restriction site, and reverse primer 5′GGGTTCCTGCGGCCGCAAAAAAATCTCGCCAACAAGTTGACG3′ (SEQ ID NO: 149) containing a Not1 site. Fragment PCR purification, digestion and a second instance of PCR purification was performed. PX441 digested backbone was purified using a Qiagen Gel Extraction kit and a ligation with both fragments was performed using T4 DNA ligase (NEB M0202). Transformation into TOP10 chemically competent cells (Invitrogen C404006) was carried out using 1 ul of unpurified ligation mix using the manufacturer's 5-minute transformation protocol. Transformed cells were grown and transfection grade plasmid prepared in triplicate using Macherey Nagel Plasmid transfection grade kit.

NGS data Generation and Frequency Analysis

10 ng of T7 template gRNA was PCR'd for 10 cycles using 5′ACACTCTTTCCCTACACGACGCTCTTCCGATCTGGGGATAATACGACTCACTATAG GC3′ (SEQ ID NO: 150) and 5′GACTGGAGTTCAGACGTGTGCTCTTCCGATCTAAAATCTCGCCAACAAGTTGACG3′ (SEQ ID NO: 151) reverse primer and sent for 2×250 paired end sequencing (Azenta). Sequencing files were processed using an in-house script.

gRNA Activity Assessment In Vitro

30 nanomoles gRNA were incubated with SaCas9 protein at a 1:1 molar ratio for 10 minutes at 37 degrees in NEB Buffer 3.1. Then, 30 nmol target DNA was added to the reaction for a total volume of 30 ul. Reaction was carried out for 30 minutes at 37 degrees and 1 ul of 800 units/ml Proteinase K (NEB P8107S) was added to terminate the reaction prior to fragment analysis in a 2% agarose gel.

gRNA activity assessment in Cells via RNPs Lipofection of RNPs was performed as previously described²⁸. Fluorescent HEK 293 cells¹⁸ were plated 24 hours prior to transfection at 15,000 cells per well in a 96 well plate in 100 ul of DMEM media (Gibco 11965118) supplemented with 10% of heat inactivated fetal bovine serum and incubated at 37 degrees Celsius at 5% CO2. For the transfection mix, 0.5 picomoles of gRNA were incubated with SaCas9 protein at a 1:1 molar ratio for 10 minutes at 37 degrees in 12.5 ul of OptiMem medium (Gibco 31985070). In a second tube, 1 ul of lipofectamine 2000 reagent (Invitrogen 11668019) was added to 12.5 ul OptiMem medium. Both tubes were mixed and incubated for 20 minutes at room temperature. 25 ul of the reaction was added to each well and GFP knockdown was assessed via flow cytometry using a Beckman Cytoflex flow cytometer 7 days post transfection. For TIDE assays, cell gDNA was extracted 72 hours post transfection using a Qiagen DNeasy Blood and Tissue kit (69504). gDNA was Nanodrop quantified and ˜100 ng was used for PCR with primers 5′ AAGGGCGAGGAGCTGTTCACC 3′ (SEQ ID NO: 152) and 5′ CGTCCTCCTTGAAGTCGATGCCC 3′ (SEQ ID NO: 153). PCR product was PCR purified, and 20 ng of amplicon, alongside with 25 pmol of primer, were sent to Azenta for Sanger sequencing.
gRNA Activity Assessment in Cells Via Plasmids

Fluorescent GFP-expressing HEK293 cells 18 were plated as detailed above. For lipofection, in a microcentrifuge tube, 0.2 ul of Lipofectamine 3000 reagent (Invitrogen L3000015) was diluted in 10 ul of OptiMem media (Gibco 31985070), while in a second tube containing 10 ul of OptiMem media, 0.1 ug of plasmid was mixed in, followed by 0.2 ul of P3000 reagent. We mixed and incubated both tubes for 10 minutes at room temperature and added to cells. After 7 days, GFP fluorescence was measured using flow cytometry.

For TIDE analysis we DNEASY (Qiagen) extracted genomic DNA after 72 hours and performed PCR on 200 ng of genomic DNA using the following primers with Q5 polymerase manufacturer protocol. For GFP we used 5′AAGGGCGAGGAGCTGTTCACC 3′ (SEQ ID NO: 154) and 5′CGTCCTCCTTGAAGTCGATGCCC3′ (SEQ ID NO: 155). For ERBB2 we used 5′GAGAACCCCGAGTACTTGACAC3′ (SEQ ID NO: 156) and 5′CTCTTGATGCCAGCAGAAGTC3′ (SEQ ID NO: 157). For KIF26B we used 5′AAAAGTCTTGAACGAAAGCTGG3′ (SEQ ID NO: 158) and 5′CAAAGACTGAGAGGACAAGCCT3′ (SEQ ID NO: 159). For MBTPS2 we used 5′CATGGGGTTACCTTTTTCAGAT3′ (SEQ ID NO: 160) and 5′TTGAACAGGTCTGACTGAGCAT3′ (SEQ ID NO: 161).

For High Throughput Sequencing genomic target analysis transfection was as follows, in a microcentrifuge tube; 0.3 ul of Lipofectamine 3000 reagent was diluted in 10 ul of OptiMem media, while in a second tube containing 10 ul of OptiMem media, 0.15 ug of plasmid was mixed in, followed by 0.3 ul of P3000 reagent. Both tubes were mixed and incubated for 10 minutes prior to addition to cells. DNA extraction proceeded as with TIDE and 500 ng of amplicon was sent for paired end sequencing (Azenta).

### Statistical Analysis

Statistical analysis of editing efficiencies was performed using Two-Tail student T tests and setting statistical significance to p<0.05.

Sequence Logo Generation and Alignment Multiple Sequence Alignment for scaffold variants were generated using Clustal Omega EBI webserver²⁹ with default settings. This was followed by generating a neighbor-joining phylogenetic tree via Simple Phylogeny³⁰, rooted around the wildtype canonical scaffold. An equiprobable sequence logo and 55% consensus sequence were generated for the multiple sequence alignment using Snapgene.

### Base Pair Probability Matrix Dot Plots

gRNA structure base pair probability matrix was predicted using the partition function of RNAfold Version 2.5.1³¹. Matrices were merged and compared using in-house scripts.

### Results

SaBLADE-Based gRNA Evolution

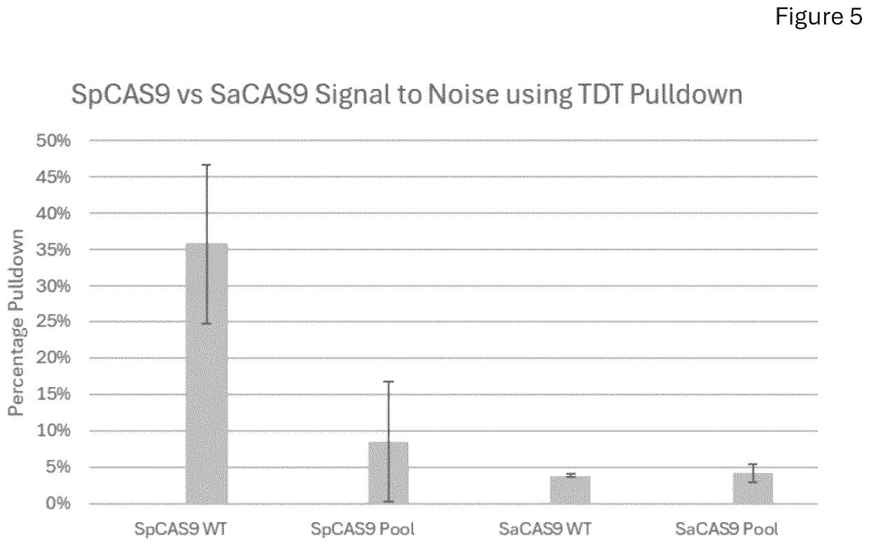

To establish a directed evolution approach to isolate functional SaCas9 gRNA variants from a randomized pool, we tested our previously described Streptococcus Pyogenes (SpCas9) gRNA BLADE method, in which terminal transferase is used to append tagged nucleotides to cleaved RNP-DNA complexes¹⁸. This however yielded poor recovery of SaCas9 gRNA compared to SpCas9 gRNA (FIG. 5). We turned to SaCas9 biochemical studies to develop an alternative BLADE approach. Unlike SpCas9 which remains bound to both DNA cleavage products for several hours after cleavage³², SaCas9 remains bound only to the PAM proximal cleavage product of the substrate DNA²⁵. We used a native polyacrylamide gel to evaluate the stability of this interaction. We observed that the SaCas9 protein could successfully capture and shift gRNA, and when added to a DNA target substrate, RNPs produced an additional gel shift, suggesting pulldown of RNP complexes via their attachment to DNA was feasible (FIG. 1A).

To quantitatively measure RNP attachment to the DNA target, we utilized ³²P radiolabeling of the gRNA to follow recovery (FIG. 1B). We radiolabeled the wildtype gRNA as a cleaving capable positive control, and separately radiolabeled non-cleaving randomized gRNA to assess background signal. We incubated SaCas9 with both radiolabeled gRNAs to form RNP complexes, then added target DNA that was end labeled with biotin at either the PAM proximal or PAM distal end. Subsequently, streptavidin magnetic beads were added to bind the biotinylated DNA targets and associated RNP complexes. Beads were pulled down and the radioactive signal of the captured RNP-DNA complex measured. Counts for wildtype gRNA (signal) were compared to randomized gRNA (background noise) and a ratio was obtained (FIG. 1B). Initial pulldown of target DNA through distal biotin labeling recovered 2% of input radioactive signal with a 1.5:1 signal to noise ratio (FIG. 1C). To increase recovery, we pre-bound DNA complexes to magnetic beads prior to RNP incubation (FIG. 1D), increasing pulldown efficiency to 50% of input using proximally labeled target, and 40% using distally labeled target, improving signal to noise ratio beyond 6:1. This improvement also raised background noise (FIG. 1D). We increased Tween20 detergent concentration to reduce nonspecific interactions from 0.002% (low) to 0.006% (high), observing reduced assay noise and a signal noise ratio over 16:1 (FIG. 1E). This established PAM proximal pulldown as a positive enrichment step. Furthermore, the 10% difference between proximal and distal pulldown fractions (FIG. 1D) is consistent with literature supporting the release of the PAM distal end of the DNA target after cleavage. Therefore, we hypothesized that PAM distal release could serve as a negative selection step. The enrichment strategy was embedded in SELEX library recovery steps to iteratively evolve SaCas9 gRNA scaffolds from a randomized gRNA pool (FIG. 1F). We termed this directed evolution strategy SaBLADE (SaCas9 Binding and ligand activated directed evolution).

SaBLADE Yields Diverse and Potent gRNAs.

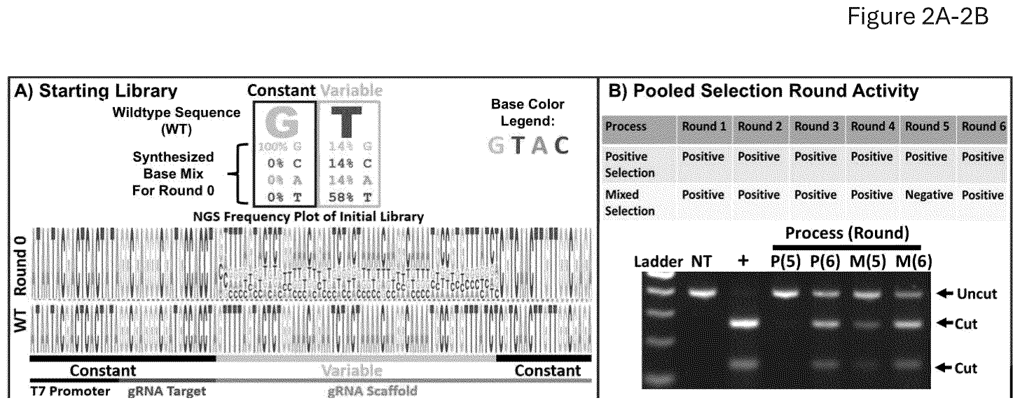

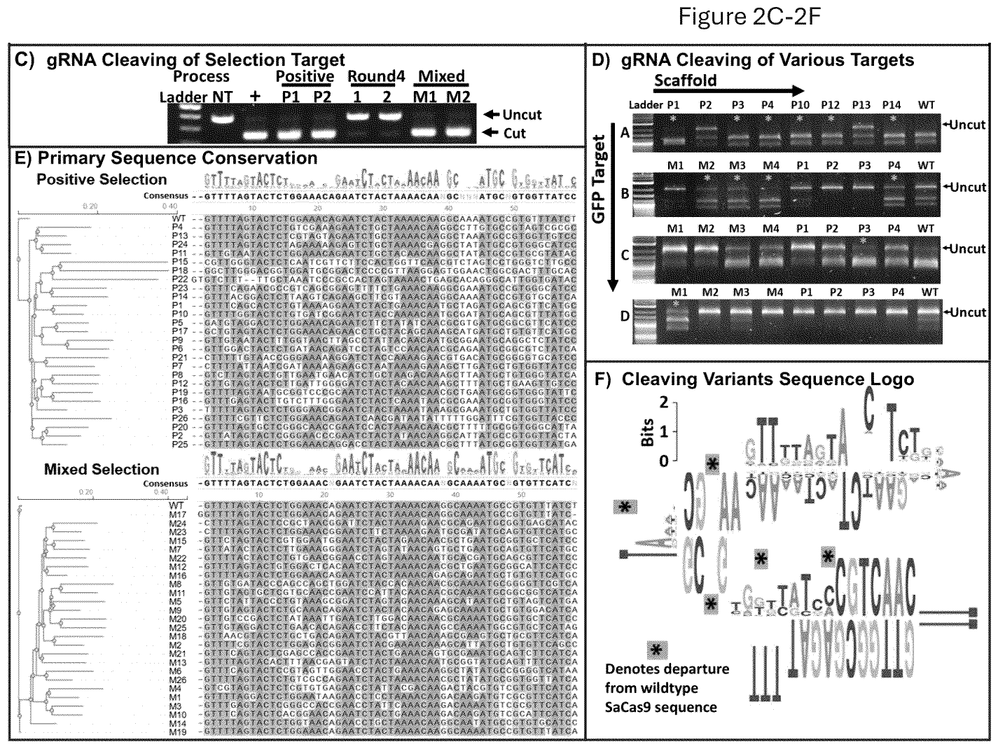

We performed SaBLADE on a partially randomized pool of gRNA variants derived from a synthesized oligonucleotide library (FIG. 2A). This library consisted of a fixed T7 promoter/DNA binding spacer, followed by a randomized repeat-anti-repeat and stem loop 1 region, and ending with a fixed scaffold stem loop 2. Fixed regions contained 100% base homogeneity, while randomized regions were synthesized using four mixtures (A,C,G,T) consisting of 58% wildtype nucleotide, and 42% mutant bases split between the remaining 3 nucleotides (14% each) at each position (FIG. 2a). We compared two SaBLADE strategies; a “Positive Selection” composed of 6 positive selection rounds and one “Mixed Selection” composed of 5 positive selection rounds and one negative selection round (FIG. 2B). We evaluated evolved gRNA pools from both strategies for RNP in vitro DNA cutting activity after one hour with a 20:1 RNP:Substrate ratio. We observed in vitro DNA cleavage using RNPs formed with pooled round 5 and round 6 gRNAs from both strategies (FIG. 2B). Furthermore, we observed that cleaving activity increased from round 5 to 6 in both cases (FIG. 2B).

We carried out multiple sequence alignment of SaBLADE gRNA variants using Clustal Omega²⁹. Variants were named using P for Positive or M for mixed selection, and the enrichment rank. We organized this data with a neighbor-joining phylogenetic tree using EBI Simple Phylogeny rooted at the canonical wildtype sequence³⁰ (FIG. 2E). Both selection strategy generated variants displayed high sequence conservation at repeat-anti repeat paired bases, consistent with prior studies suggesting the importance of this region^{1,5,11,26,33}. We however observed sequence flexibility around the artificial repeat-anti repeat tetraloop.^{1,5,26}.

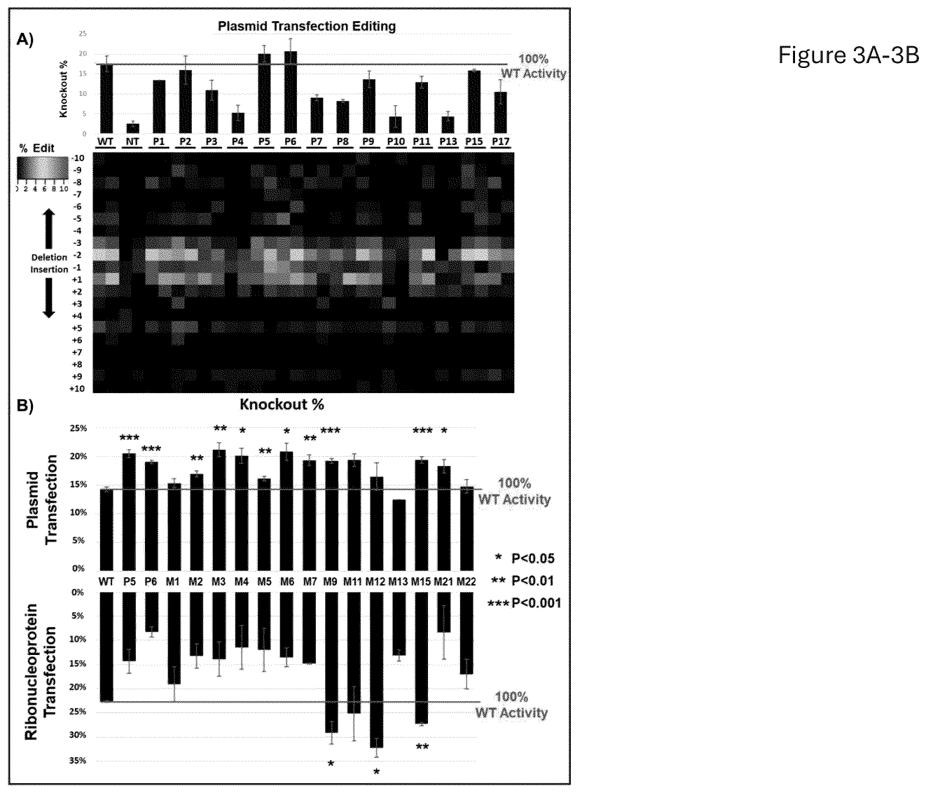

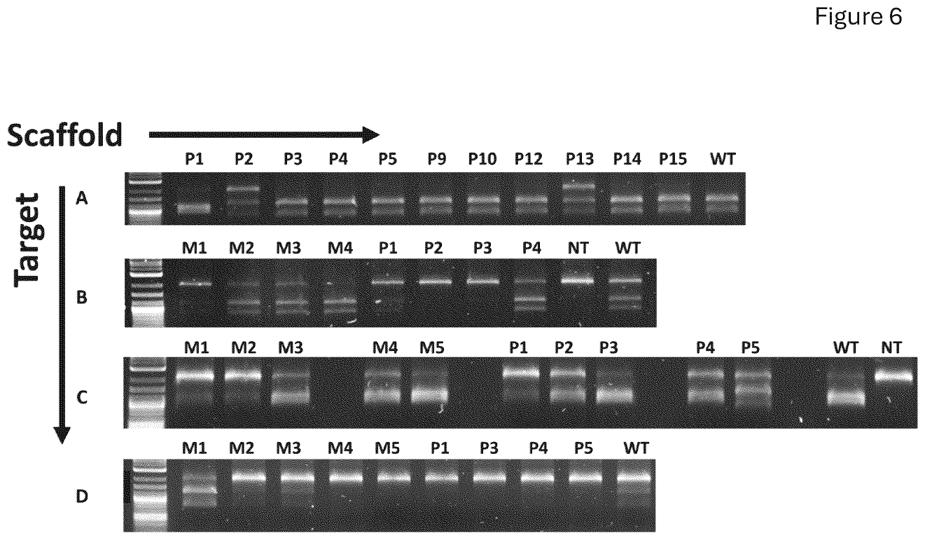

We evaluated gRNAs individually for DNA cleavage of the sequence utilized in SaBLADE. We observed effective in vitro cleavage with variants obtained after six rounds of both selection strategies, and minimal activity from round 4 gRNA variants (FIG. 2C). Next, we tested cleaving at 4 new GFP DNA targets, and obtained improved in vitro activity over wildtype scaffold using several target-scaffold pairs (FIG. 2D, FIG. 6). While no single scaffold variant outperformed wildtype gRNA scaffold at all targets, different scaffolds showed comparable or improved activity over wildtype at each target. DNA target A was cleaved to completion by several scaffolds, and we utilized it to further characterize variant gRNAs editing efficiencies in GFP+ cells (FIG. 3A, B).

We plotted a summary sequence logo of variants with in-vitro cleaving activity arranged according to the known secondary structure of SaCas9 gRNA^{5,26}, and found that interacting bases had similar rates of sequence conservation (FIG. 2F). Furthermore, we observed that 5 bases evolved away from the wildtype scaffold primary sequence.

SaBLADE-Derived gRNAs can Improve Cellular Gene Editing Efficiency.

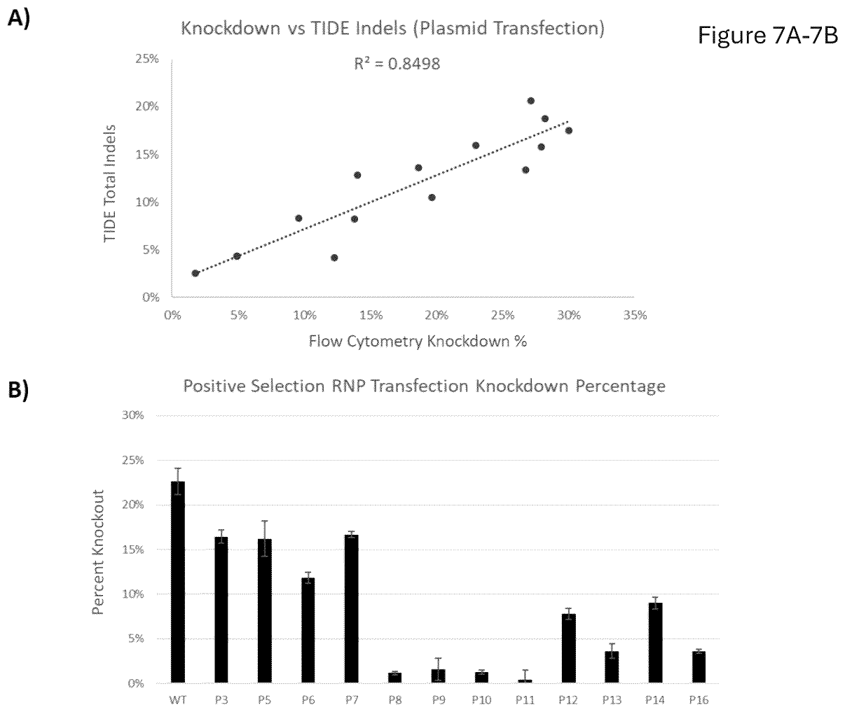

To test the nuclease activity of SaCas9 variants in cells, we transfected px441 plasmid, expressing gRNA variants using a U6 promoter, and SaCas9 using a cytomegalovirus (CMV) promoter. We extracted genomic DNA after 72 hours, amplified the GFP target via PCR, and performed sanger-based trace decomposition analysis (TIDE) 34 to generate total editing rates (upper bar graph) and indel heatmaps (FIG. 3A). The wildtype gRNA scaffold outperformed most positive selection variants tested except for P5 and P6. Indels were concentrated between −3 deletions and +1 insertions for all scaffolds regardless of activity level. Next, we tested the suitability of flow cytometry GFP fluorescent knockout to measure gene editing efficiency 7 days post transfection as previously described 18. When comparing TIDE and knockout data, TIDE derived indel frequencies were highly correlated (R\hat{ }=0.84) with GFP fluorescence knockout efficiencies (FIG. 7A). We also found that GFP knockout efficiencies from positive selection variants did not surpass those of the wildtype scaffold (FIG. 7B). By contrast some mixed selection scaffolds enhanced GFP knockout editing efficiency up to 139% of wildtype gRNA scaffold (bottom of FIG. 3B). Mixed selection scaffolds also performed well following plasmid transfection, with several variants significantly enhancing SaCas9 activity (top of FIG. 3B). Next, we evaluated if gRNA variants could improve the editing efficiency of endogenous genomic targets that are resistant to editing by the wildtype SaCas9 gRNA scaffold.

SaBLADE-Derived gRNAs Significantly Improve Editing at Difficult Genomic Targets.

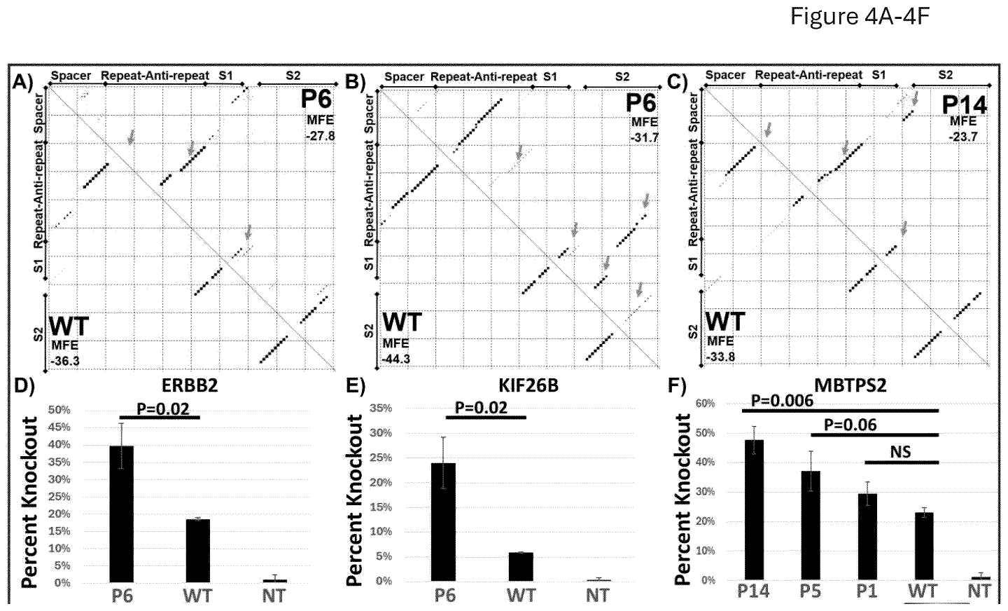

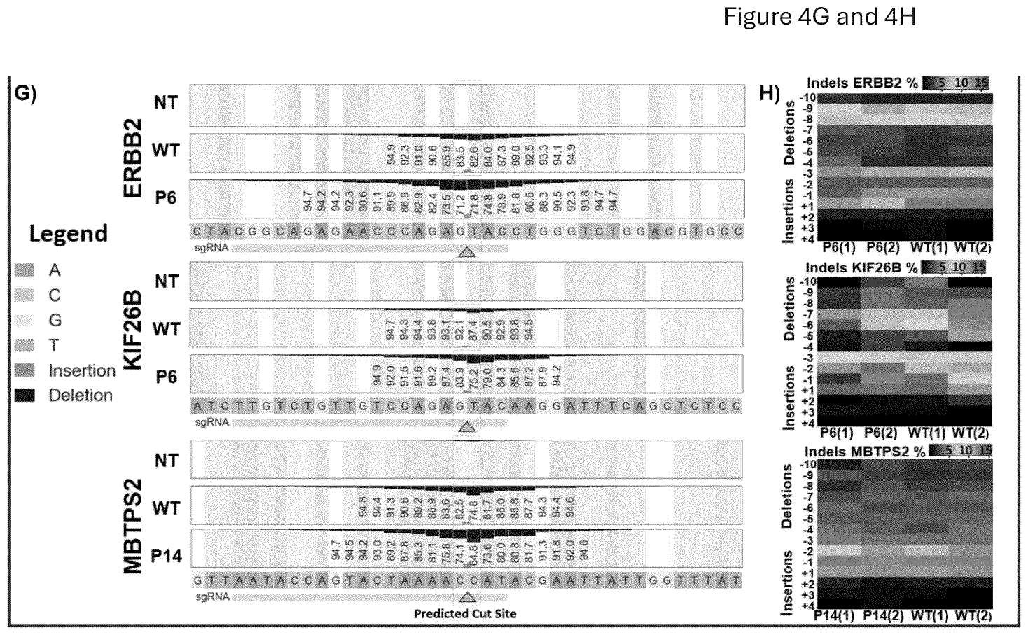

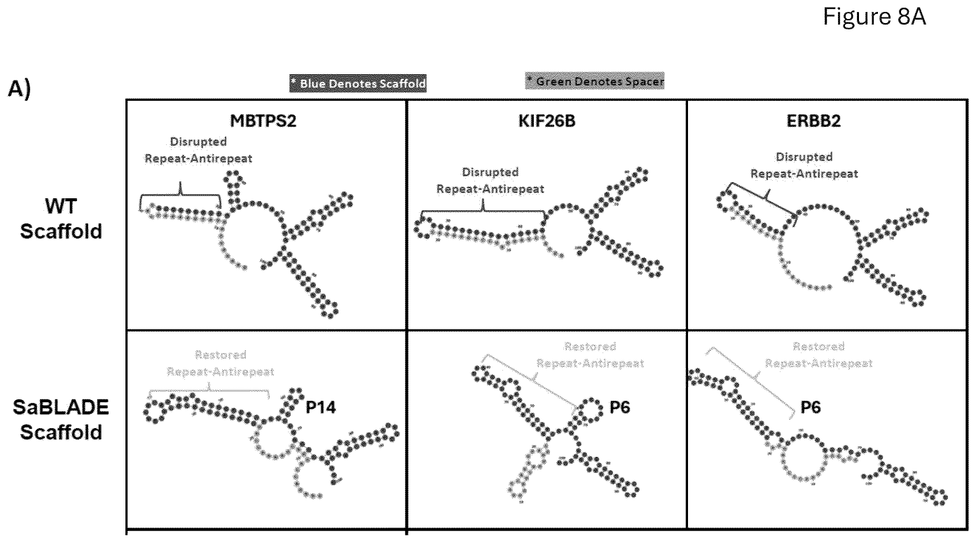

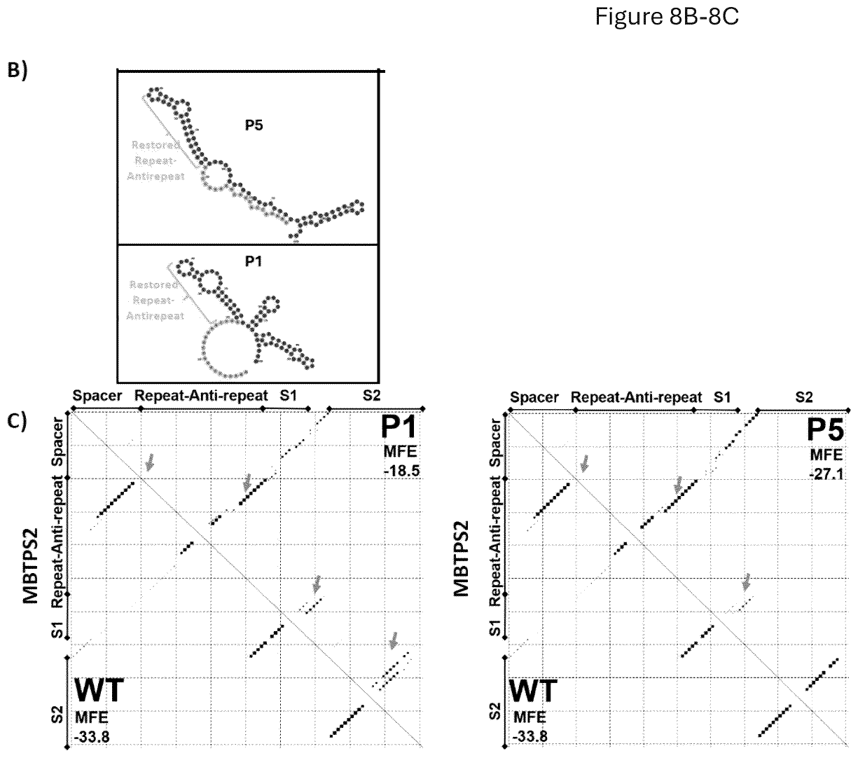

To identify gene targets with low editing efficiency using the wildtype SaCas9 gRNA, we performed an NCBI blast search for nucleotide patterns which disrupted canonical guide RNA scaffold motifs^{1,8,10,14,15,33}. We identified spacers targeting ERBB2, KIF26B, and MBTPS2 complementary to the repeat-anti-repeat region of the gRNA³⁵⁻⁴⁰. We used RNAfold RNA minimum free energy (MFE) model to visualize these interactions, with wild-type scaffold forming base pairs between the spacer and the repeat-anti-repeat, disrupting its canonical self-pairing. In contrast, SaBLADE scaffolds mitigated this interaction, preserving the native repeat-anti-repeat pairing and overall structural integrity (FIG. 8A-B). To observe a more comprehensive picture of secondary structure, we compared the dotplots of SaBLADE (top right triangle) to wildtype (lower left triangle) scaffolds for ERBB2 (4A), KIF26B (4B) and MBTPS2 (4C, 8C-D), with arrows denoting structural changes between variant and wildtype scaffolds. Key structural gRNA elements from the literature are annotated for interpretability²⁶. We observed important structural changes in all cases (FIG. 4A-C, and FIG. 8C,D), including the shortening stem loop 1 back to its canonical length in all cases, and the regaining of repeat anti-repeat canonical folding for ERBB2 and MBTPS2. KIF26B, despite having an MFE with restored repeat/anti-repeat structure (FIG. 8A), only had minimal improvement in this structure.

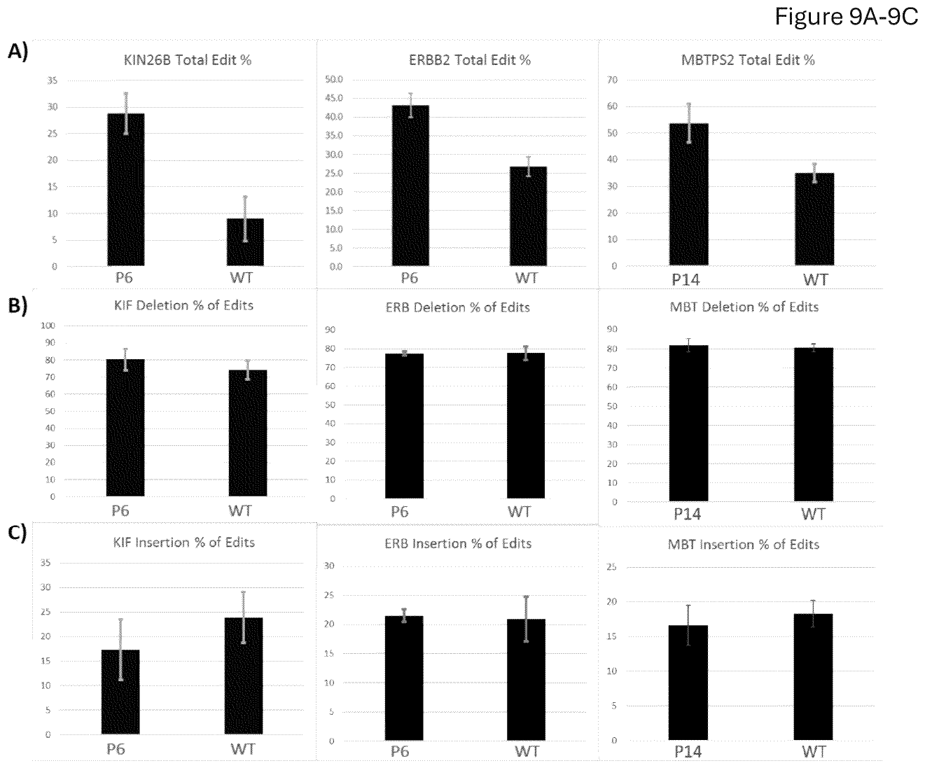

We transfected 0.1 ug of plasmid containing variant and wildtype gRNAs paired to these targets into HEK293 cells in 96 well plates. Sanger Sequencing Trace Decomposition (TIDE) was used to evaluate editing in ERBB2 (FIG. 4D), KIF26B (FIG. 4E) and MBTPS2 (FIG. 4F). Variant scaffolds significantly improved indel percentages by 206%, 412%, and 214% respectively (FIG. 4D-F). We further characterized these results using CRISPResso2⁴¹ to analyze paired end next generation sequencing data. To determine if this improvement in editing efficiency was maintained at higher doses of gRNA, we evaluated a higher plasmid dose (0.15 ug/well). This experiment maintained or improved editing at all targets using variant SaBLADE gRNAs with improvements over wildtype gRNA-based editing remaining statistically significant (FIG. 9A). The distribution of base deletions formed a bell curve around the predicted cut site for all guide RNA scaffolds and all genomic targets. (FIG. 4G). Quantification of the contribution of deletions and insertion events to total editing events showed no significant differences between scaffolds grouped by target (FIG. 9B-C). For each given target, wildtype and SaBLADE variant scaffolds also shared similar, target specific distribution of total indel size, with ERBB2 indel sizes mostly falling between −9 deletion and +1 insertion, KIF26B indel sizes between −7 deletion and +1 insertion, and MBTPS2 indel sizes between −5 deletion and +1 insertion (FIG. 4H). Therefore, SaBLADE scaffolds had similar indel distribution and indel size in cells to wildtype, while significantly improving activity at difficult to edit human genome targets.

### DISCUSSION

We find SaBLADE evolution on a ˜1E14 initial library of SaCas9 gRNA variants uncovered an arsenal of SaCas9 gRNA scaffolds that improve editing efficiency and discern structure-function relationships in the gRNA scaffold region of SaCas9. We find that gRNA scaffold variants yield the greatest improvement in editing efficacy at difficult target sequences, maximizing activity rescue where it is most needed. The positive and mixed selection strategies we describe, add an approach to evolve gRNAs for any Cas9 ortholog possessing asymmetric post-cleaving target dissociation⁴²⁻⁴⁴. With current libraries of natural CRISPR operons reaching hundreds of thousands 45, the number of Cas9 orthologs to be explored is constantly growing.

In-vitro evolution allows for testing of over 1E14 initial scaffold variants. These numbers are 7 to 10 orders of magnitude higher than what is currently feasible to explore with bacterial⁵³ and mammalian⁵⁴ high throughput screens. One can however envision pipelines that combine in-vitro evolution, bacterial evolution and mammalian screens, as the latter two present the advantage of testing protein variants simultaneously in addition to gRNAs. For instance, in-vitro evolution gRNA libraries leading to hundreds of functional enriched hits, would be ideal to pre-diversify libraries for Phage Assisted Continuous Evolution (PACE)⁵⁵. Furthermore, screens that combine gRNA variants with protein variants using mammalian lentiviral screens⁵⁴, represent an exciting orthogonal approach to explore co-evolutionary relationships between Cas proteins and gRNAs.

While SaBLADE scaffold evolution targeting a GFP site yielded variants that improve the editing activity of SaCas9 at genomic targets, we were unable to test most scaffolds generated from selection. Performing high throughput testing of variants at multiple DNA target sites will be essential to generate data of the scale necessary to train machine learning and language models. Furthermore, this study evolved scaffolds to one GFP target, which were successful at editing alternate targets. Therefore, one can envision BLADE evolution at new targets will generate scaffold variants further specialized for the explored substrates. In-vitro directed evolution methods, while still in their infancy, hold great promise to help accelerate the development of gene editors at unprecedented scales.

### REFERENCES

1. Jinek M, Chylinski K, Fonfara I, et al. A Programmable Dual-RNA-Guided DNA Endonuclease in Adaptive Bacterial Immunity. Science 2012; 337(6096):816-821; doi: 10.1126/science.1225829.
2. Villiger L, Joung J, Koblan L, et al. CRISPR technologies for genome, epigenome and transcriptome editing. Nat Rev Mol Cell Biol 2024; 25(6):464-487; doi: 10.1038/s41580-023-00697-6.
3. Yao R, Liu D, Jia X, et al. CRISPR-Cas9/Cas12a biotechnology and application in bacteria. Synth Syst Biotechnol 2018; 3(3):135-149; doi: 10.1016/j.synbio.2018.09.004.
4. Sullenger B A. RGEN Editing of RNA and DNA: The Long and Winding Road from Catalytic RNAs to CRISPR to the Clinic. Cell 2020; 181(5):955-960; doi: 10.1016/j.cell.2020.04.050.
5. Ran F A, Cong L, Yan W X, et al. In vivo genome editing using Staphylococcus aureus Cas9. Nature 2015; 520(7546):186-191; doi: 10.1038/nature14299.
6. Xiang X, Corsi G I, Anthon C, et al. Enhancing CRISPR-Cas9 gRNA efficiency prediction by data integration and deep learning. Nat Commun 2021; 12(1):3238; doi: 10.1038/s41467-021-23576-0.
7. Yang Z-X, Fu Y-W, Zhao J-J, et al. Superior Fidelity and Distinct Editing Outcomes of SaCas9 Compared with SpCas9 in Genome Editing. Genom, Proteom Bioinform 2022; 21(6):1206-1220; doi: 10.1016/j.gpb.2022.12.003.
8. Chari R, Yeo N C, Chavez A, et al. sgRNA Scorer 2.0: A Species-Independent Model To Predict CRISPR/Cas9 Activity. ACS Synth Biol 2017; 6(5):902-904; doi: 10.1021/acssynbio.6b00343.
9. Moreno-Mateos M A, Vejnar C E, Beaudoin J-D, et al. CRISPRscan: designing highly efficient sgRNAs for CRISPR-Cas9 targeting in vivo. Nat Methods 2015; 12(10):982-988; doi: 10.1038/nmeth.3543.
10. Labun K, Montague T G, Krause M, et al. CHOPCHOP v3: expanding the CRISPR web toolbox beyond genome editing. Nucleic Acids Res 2019; 47(W1):W171-W174; doi: 10.1093/nar/gkz365.
11. Hanna R E, Doench J G. Design and analysis of CRISPR-Cas experiments. Nat Biotechnol 2020; 38(7):813-823; doi: 10.1038/s41587-020-0490-7.
12. Hall B, Cho A, Limaye A, et al. Genome Editing in Mice Using CRISPR/Cas9 Technology. Curr Protoc Cell Biol 2018; 81(1):e57; doi: 10.1002/cpcb.57.
13. Xu K, Segal D J, Zhang Z. Editorial: Precise Genome Editing Techniques and Applications. Front Genet 2020; 11:412; doi: 10.3389/fgene.2020.00412.
14. Thyme S B, Akhmetova L, Montague T G, et al. Internal guide RNA interactions interfere with Cas9-mediated cleavage. Nat Commun 2016; 7(1):11750; doi: 10.1038/ncomms11750.
15. Okafor I C, Ha T. Single Molecule FRET Analysis of CRISPR Cas9 Single Guide RNA Folding Dynamics. J Phys Chem B 2023; 127(1):45-51; doi: 10.1021/acs.jpcb.2c05428.
16. Xu X, Duan D, Chen S-J. CRISPR-Cas9 cleavage efficiency correlates strongly with target-sgRNA folding stability: from physical mechanism to off-target assessment. Sci Rep-uk 2017; 7(1):143; doi: 10.1038/s41598-017-00180-1.
17. Riesenberg S, Helmbrecht N, Kanis P, et al. Improved gRNA secondary structures allow editing of target sites resistant to CRISPR-Cas9 cleavage. Nat Commun 2022; 13(1):489; doi: 10.1038/s41467-022-28137-7.
18. Bush K, Corsi G I, Yan A C, et al. Utilizing directed evolution to interrogate and optimize CRISPR/Cas guide RNA scaffolds. Cell Chem Biol 2023; doi: 10.1016/j.chembiol.2023.06.007.
19. Ryczek M, Pluta M, Blaszczyk L, et al. Overview of Methods for Large-Scale RNA Synthesis. Appl Sci 2022; 12(3):1543; doi: 10.3390/app12031543.
20. Reese C B. Oligo- and poly-nucleotides: 50 years of chemical synthesis. Org Biomol Chem 2005; 3(21):3851-3868; doi: 10.1039/b510458k.
21. Flamme M, McKenzie L K, Sarac I, et al. Chemical methods for the modification of RNA. Methods 2019; 161:64-82; doi: 10.1016/j.ymeth.2019.03.018.
22. Nimjee S M, White R R, Becker R C, et al. Aptamers as Therapeutics. Annu Rev Pharmacol Toxicol 2017; 57(1):61-79; doi: 10.1146/annurev-pharmtox-010716-104558.
23. Stoltenburg R, Reinemann C, Strehlitz B. SELEX-A (r)evolutionary method to generate high-affinity nucleic acid ligands. Biomol Eng 2007; 24(4):381-403; doi: 10.1016/j.bioeng.2007.06.001.
24. Wilbanks B, Pearson K, Maher L J. A non-rational approach to optimized targeting of CRISPR-Cas9 complexes. Cell Chem Biol 2023; 30(8):855-857; doi: 10.1016/j.chembiol.2023.07.012.
25. Zhang S, Zhang Q, Hou X, et al. Dynamics of Staphylococcus aureus Cas9 in DNA target Association and Dissociation. EMBO Rep 2020; 21(10):e50184; doi: 10.15252/embr.202050184.
26. Nishimasu H, Cong L, Yan W X, et al. Crystal Structure of Staphylococcus aureus Cas9. Cell 2015; 162(5):1113-1126; doi: 10.1016/j.cell.2015.08.007.
27. Zhang Y, Nishiyama T, Li H, et al. A consolidated AAV system for single-cut CRISPR correction of a common Duchenne muscular dystrophy mutation. Mol Ther Methods Clin Dev 2021; 22:122-132; doi: 10.1016/j.omtm.2021.05.014.
28. Wang Y, Wang B, Xie H, et al. Efficient Human Genome Editing Using SaCas9 Ribonucleoprotein Complexes. Biotechnol J 2019; 14(7):1800689; doi: 10.1002/biot.201800689.
29. Sievers F, Wilm A, Dineen D, et al. Fast, scalable generation of high-quality protein multiple sequence alignments using Clustal Omega. Mol Syst Biol 2011; 7(1):MSB201175; doi: 10.1038/msb.2011.75.
30. Madeira F, Madhusoodanan N, Lee J, et al. The EMBL-EBI Job Dispatcher sequence analysis tools framework in 2024. Nucleic acids Res 2024; 52(W1):W521-W525; doi: 10.1093/nar/gkae241.
31. Lorenz R, Bernhart S H, Siederdissen C H Z, et al. ViennaRNA Package 2.0. Algorithms Mol Biol: AMB 2011; 6(1):26; doi: 10.1186/1748-7188-6-26.
32. Yourik P, Fuchs R T, Mabuchi M, et al. Staphylococcus aureus Cas9 is a multiple-turnover enzyme. Rna 2019; 25(1):35-44; doi: 10.1261/rna.067355.118.
33. Labun K, Montague T G, Gagnon J A, et al. CHOPCHOP v2: a web tool for the next generation of CRISPR genome engineering. Nucleic Acids Res 2016; 44(Web Server issue):W272-W276; doi: 10.1093/nar/gkw398.
34. Brinkman E K, Chen T, Amendola M, et al. Easy quantitative assessment of genome editing by sequence trace decomposition. Nucleic Acids Res 2014; 42(22):e168-e168; doi: 10.1093/nar/gku936.
35. Valtorta E, Martino C, Sartore-Bianchi A, et al. Assessment of a HER2 scoring system for colorectal cancer: results from a validation study. Mod Pathol 2015; 28(11):1481-1491; doi: 10.1038/modpathol.2015.98.
36. Lu M, Wang T, He M, et al. Tumor suppressor role of miR-3622b-5p in ERBB2-positive cancer. Oncotarget 2017; 8(14):23008-23019; doi: 10.18632/oncotarget.14968.
37. Yamamura Y, Iwata Y, Furuichi K, et al. Kif26b contributes to the progression of interstitial fibrosis via migration and myofibroblast differentiation in renal fibroblast. FASEB J 2022; 36(11):e22606; doi: 10.1096/fj.202200355r.
38. Li H, Shen S, Chen X, et al. miR-450b-5p loss mediated KIF26B activation promoted hepatocellular carcinoma progression by activating PI3K/AKT pathway. Cancer Cell Int 2019; 19(1):205; doi: 10.1186/s12935-019-0923-x.
39. Tibbo A J, Hartley A, Vasan R, et al. MBTPS2 acts as a regulator of lipogenesis and cholesterol synthesis through SREBP signalling in prostate cancer. Br J Cancer 2023; 128(11):1991-1999; doi: 10.1038/s41416-023-02237-7.
40. Caengprasath N, Theerapanon T, Porntaveetus T, et al. MBTPS2, a membrane bound protease, underlying several distinct skin and bone disorders. J Transl Med 2021; 19(1):114; doi: 10.1186/s12967-021-02779-5.
41. Clement K, Rees H, Canver M C, et al. CRISPResso2 provides accurate and rapid genome editing sequence analysis. Nat Biotechnol 2019; 37(3):224-226; doi: 10.1038/s41587-019-0032-3.
42. Wang S, Mao H, Hou L, et al. Compact SchCas9 Recognizes the Simple NNGR PAM. Adv Sci (Weinh, Baden-Wurttemberg, Ger) 2021; 9(4):e2104789; doi: 10.1002/advs.202104789.
43. Hu Z, Zhang C, Wang S, et al. Discovery and engineering of small SlugCas9 with broad targeting range and high specificity and activity. Nucleic Acids Res 2021; 49(7):4008-4019; doi: 10.1093/nar/gkab148.
44. Wang S, Tao C, Mao H, et al. Identification of SaCas9 orthologs containing a conserved serine residue that determines simple NNGG PAM recognition. PLOS Biol 2022; 20(11):e3001897; doi: 10.1371/journal.pbio.3001897.
45. Ruffolo J A, Nayfach S, Gallagher J, et al. Design of highly functional genome editors by modeling the universe of CRISPR-Cas sequences. bioRxiv 2024; 2024.04.22.590591; doi: 10.1101/2024.04.22.590591.
46. Xie H, Ge X, Yang F, et al. High-fidelity SaCas9 identified by directional screening in human cells. PLOS Biol 2020; 18(7):e3000747; doi: 10.1371/journal.pbio.3000747.
47. Tan Y, Chu A H Y, Bao S, et al. Rationally engineered Staphylococcus aureus Cas9 nucleases with high genome-wide specificity. Proc Natl Acad Sci 2019; 116(42):20969-20976; doi: 10.1073/pnas.1906843116.
48. Yuen C T L, Thean D G L, Chan B K C, et al. High-fidelity KKH variant of Staphylococcus aureus Cas9 nucleases with improved base mismatch discrimination. Nucleic Acids Res 2022; 50(3):1650-1660; doi: 10.1093/nar/gkab1291.
49. Luan B, Xu G, Feng M, et al. Combined Computational-Experimental Approach to Explore the Molecular Mechanism of SaCas9 with a Broadened DNA Targeting Range. J Am Chem Soc 2019; 141(16):6545-6552; doi: 10.1021/jacs.8b13144.
50. Thean D G L, Chu H Y, Fong J H C, et al. Machine learning-coupled combinatorial mutagenesis enables resource-efficient engineering of CRISPR-Cas9 genome editor activities. Nat Commun 2022; 13(1):2219; doi: 10.1038/s41467-022-29874-5.
51. Schmidt M J, Gupta A, Bednarski C, et al. Improved CRISPR genome editing using small highly active and specific engineered RNA-guided nucleases. Nat Commun 2021; 12(1):4219; doi: 10.1038/s41467-021-24454-5.
52. Nguyen E, Poli M, Durrant M G, et al. Sequence modeling and design from molecular to genome scale with Evo. Science 2024; 386(6723):eado9336; doi: 10.1126/science.ado9336.
53. Miller S M, Wang T, Liu D R. Phage-assisted continuous and non-continuous evolution. Nat Protoc 2020; 15(12):4101-4127; doi: 10.1038/s41596-020-00410-3.
54. Wang T, Lander E S, Sabatini D M. Viral Packaging and Cell Culture for CRISPR-Based Screens. Cold Spring Harb Protoc 2016; 2016(3):pdb.prot090811; doi: 10.1101/pdb.prot090811.
55. Esvelt K M, Carlson J C, Liu D R. A system for the continuous directed evolution of biomolecules. Nature 2011; 472(7344):499-503; doi: 10.1038/nature09929.

### Example 2: BLADE Directed Evolution of SaCas9 gRNAs

CRISPR-based editing is inefficient at over two thirds of genetic targets. A primary cause is RNA misfolding that can occur between the spacer and scaffold regions of the gRNA, which hinders the formation of functional Cas9 ribonucleoprotein complexes (RNPs). Here, we uncover hundreds of highly efficient gRNA variant scaffolds for Staphylococcus aureus (Sa)Cas9 utilizing an innovative BLADE (Binding and Ligand Activation Driven Enrichment) methodology provided in Example 1, which leverages asymmetric product dissociation over rounds of evolution. SaBLADE-derived gRNA scaffolds contain 7-42% of nucleotide variation relative to wildtype. gRNA variants are able to improve gene editing efficiency at all targets tested, and they achieve their highest levels of editing improvement (>400%) at the most challenging DNA target sites for the wildtype SaCas9 gRNA. This arsenal of SaBLADE-derived gRNA variants showcases the power and flexibility of combinatorial chemistry and directed evolution to enable efficient gene editing at challenging, or previously intractable, genomic sites.

### High Throughput Activity Assay

### Materials and Methods

Overview: Activity assays bead based (see Example 1), and gel shift (method 2 and 3) were performed with Initial scaffold gRNA library (method 1). Then multiplex activity gel shift assay (method 6), was performed with Scaffold gRNA library (method 4), and target library (method 5) for multiple targets.
1. Initial Scaffold Pooled gRNA Library Generation

For our proof-of-concept study we used a 150-gRNA scaffold variant library+Wildtype scaffold, including 134 new variants reported herein and 16 variants reported in WO2022197727A9. This library was synthesized with a 5′ phosphorylated end. Given the common final 23 nucleotides present in all gRNA variants, we were able to use a reverse complement primer to initiate a Klenow reaction, resulting in a dsDNA scaffold library. We proceeded to ligate this library using T4 ligase to the annealed Target A GFP sequence obtained from primers 5′ CCGGTGGTGCAGATGAACTT 3′ (SEQ ID NO: 350) and 5′ AAGTTCATCTGCACCACCGG 3′ (SEQ ID NO: 351). This was then amplified using forward primer 5′ GGGGATAATACGACTCACTATAGCCGGTGGTGCAGATGAACTT 3′ (SEQ ID NO: 352) containing the T7 promoter and reverse primer 5′ AAAATCTCGCCAACAAGTTGACG 3′ (SEQ ID NO: 136), allowing for its subsequent T7 transcription into gRNAs, which were polyacrylamide gel purified, and ethanol precipitated to remove salts. This pool was analyzed using a Nanodrop 3000 to obtain its concentration and ensure high final purity measurements.

2. saBLADE Bead-Based Activity Assay

For one round positive selection, assay conditions were the same as those of a single round of SaCAS9 Directed Evolution. We began with RNP formation between the bulk library of transcribed gRNAs and SaCas9 protein in equimolar ratio (0.2 nmol) for 10 minutes at room temperature. Meanwhile, the DNA target was amplified from a GFP mutant sequence containing the target sequence 5′GGCGAGGGCGATGCCACCTA3′ (SEQ ID NO: 138) followed immediately by the PAM sequence 5′ CGGAAT 3′ (SEQ ID NO: 139) amplified using forward primer 5′ GTGAGCAAGGGCGAGGAGCTG3′ (SEQ ID NO: 140) and reverse primer 5′ TACTTGTACAGCTCGTCCATGC 3′ (SEQ ID NO: 141), with the reverse primer bearing 5′ biotin (PAM proximal Positive Selection). This target was pre-incubated with biotinylated beads for 1 hour and beads were added to the RNP mixture after 3 washes with 1 ml NEB Buffer 3.1. Total DNA targets within the beads were added in a 10-fold molar deficit relative to RNP concentration, totaling 0.02 nmol.

Phenol/chloroform/isoamyl alcohol 25:24:1 (Invitrogen 15593049) was added to the sample, thoroughly vortexed for 1 minute and spun at 21,000×G to separate phases, and the aqueous phase containing RNA was retained. Ethanol precipitation was performed using 10% volume of 3M sodium acetate, 3 ul of linear acrylamide (Thermo Fisher AM9520) and 3 volumes of 100% ethanol, followed by incubation at −80 degrees for 10 minutes. A 30 minute 21000×G centrifuge spin was followed by two 70% ethanol washes followed by pellet drying and reconstitution. This sample was subsequently reverse transcribed using MMLV RT and an SaCas9 reverse primer 5′ AAAATCTCGCCAACAAGTTGACG 3′ (SEQ ID NO: 136) per manufacturer protocol (Thermo Fisher 28025013) PCR was carried out to reamplify the enriched DNA library using 5′ GGGGATAATACGACTCACTATAGGCGAGGGCGATGCCACCTA 3′ (SEQ ID NO: 146) and 5′ AAAATCTCGCCAACAAGTTGACG 3′ (SEQ ID NO: 136). Enriched gRNA obtained was compared using log fold change relative to the starting transcribed gRNA library, measured through the reverse transcription of the library using reverse primer 5′ AAAATCTCGCCAACAAGTTGACG 3′ (SEQ ID NO: 136) and PCR amplified to obtain the enriched DNA library using SEQ ID NO: 146 and SEQ ID NO: 136.

3. saBLADE Gel Shift Activity Assay (Single Target)

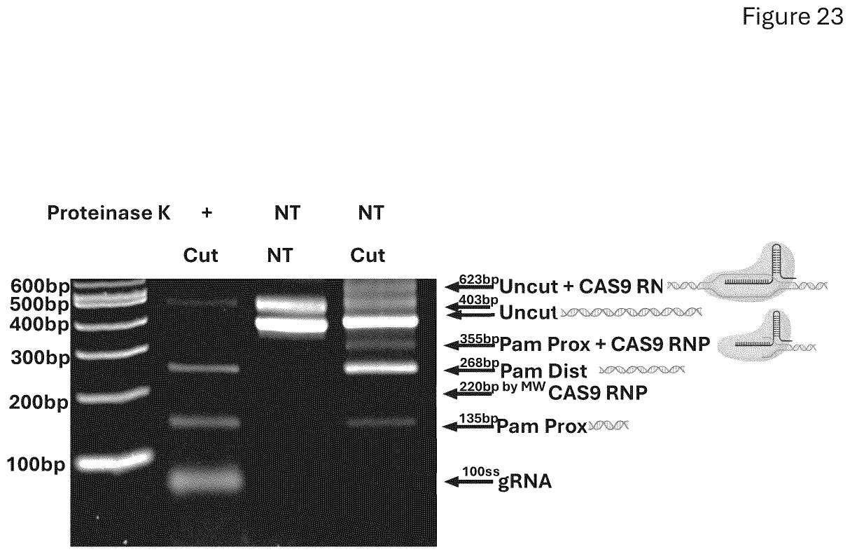

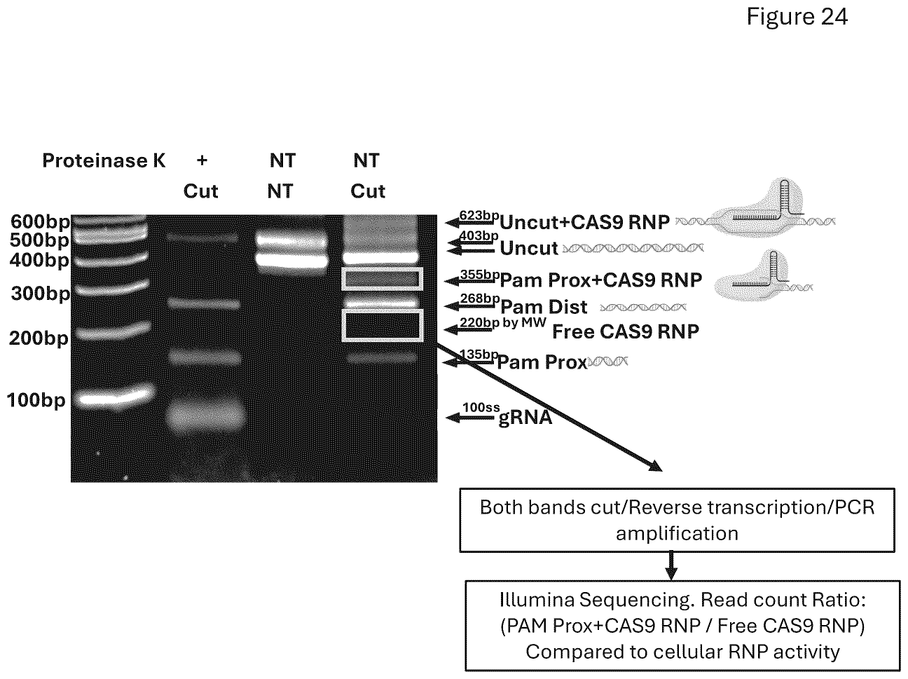

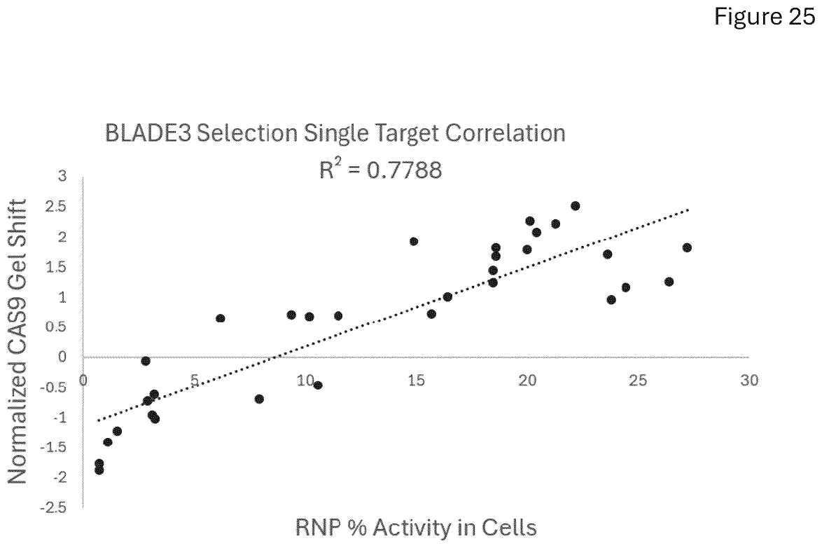

Cas9 protein and gRNA were added together at a 1:1 molar ratio using 200 ng of pooled gRNA described above and 5 pmol of saCAS9 protein. After 10 minutes of incubation, 1.35 ug of target DNA was added and incubated for 10-minutes at 37 degrees. The reaction was added to a Purple Loading Dye lacking SDS (NEB Cat #B7025S) with supplemented 20 mM MgCl2, and run in an unstained native 4% agarose gel for 30 minutes at 4 degrees Celsius. Prior to being cast, the gel was supplemented to a final 20 mM MgCl2 concentration to provide excess magnesium, negating EDTA chelation present in the native gel. Running buffer was also set to contain a final 20 mM MgCl2 concentration and was chilled to 4 degrees prior to running the gel. The gels are shown in FIG. 23. The PAM proximal band, gel shifted by Cas9˜350 bp equivalent, and the free RNP (˜220 bp equivalent), were extracted (See FIG. 24), crushed and soaked in TE buffer overnight to allow for diffusion of complexes outside the gel. The samples were then phenol chloroform extracted and ethanol precipitated into a low volume. We proceeded to reverse transcription using the universal reverse transcription primer 5′AAAATCTCGCCAACAAGTTGACG 3′ (SEQ ID NO: 136). The sample was then amplified using forward primer 5′CCGGTGGTGCAGATGAACTT3′ and reverse primer 5′AAAATCTCGCCAACAAGTTGACG 3′ (SEQ ID NO: 136) and sent for high throughput sequencing. A ratio was obtained between the PAM proximal Cas9 bound fraction collected normalized to free RNPs. We carried out standard pre-processing including low quality score filtering, read ends trimming, forward and reverse read alignment and paired read joining. Using read counts tallied by scaffold, we calculated the Log Fold Change (LFC) between the Pam Proximal cut band and the free RNP cut band. We plotted this ratio against our existing cell activity RNP based lipofection data for GFP (see SEQ ID NOs: 292-345, these sequences are composed of the GFP target “gCCGGTGGTGCAGATGAACTT” (SEQ ID NO: 225) followed by a gRNA scaffold as provided in SEQ ID NO 1-134 also see FIG. 25).

3. Scaffold Pooled gRNA Library for Multiple Target Generation

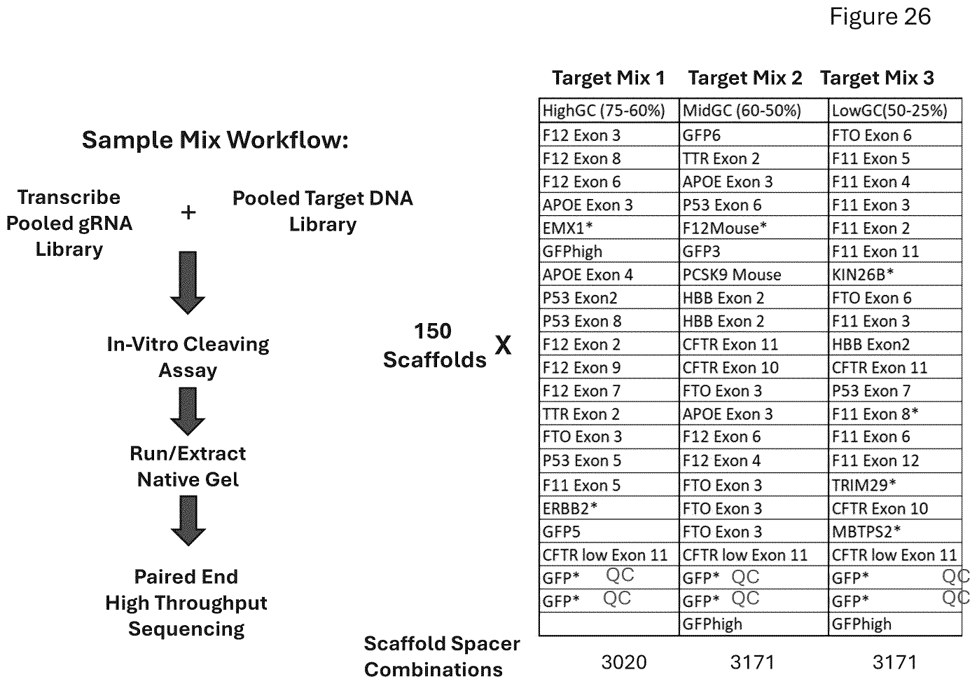

We combined the sequences of our 150-scaffold library to 57 spacer targets, divided by GC content into 3 libraries, generating 8607 combinations. Spacer targets were derived from genomic regions with clinical relevance (Provided in SEQ ID NOs: 162-182, 204-225, and 248-269). These were synthesized as one pooled library which from 5′ to 3′ included a 22 base adaptor sequence for amplification, the T7 polymerase promoter (18 bases), followed by the sgRNA culminating in TTTT. Given the common final 23 nucleotides present in all gRNA variants, we were able to use a reverse complement primer to initiate a Klenow reaction, resulting in a dsDNA scaffold library. A common reverse primer 5′ AAAATCTCGCCAACAAGTTGACG 3 (SEQ ID NO: 136) was used separately combining it to one of three adaptor sequences were used to selectively PCR amplify this library into three groups consisting of high gc content gRNAs+controls (SEQ ID NOs: 162-182 and 183-203), medium gc content gRNAs+controls (SEQ ID NOs: 204-225 and 226-247) and low gc content gRNAs+ controls (SEQ ID NOs: 248-269 and 270-291). See FIG. 26. Each library was T7 transcribed into gRNAs, which were then polyacrylamide gel purified, and ethanol precipitated to remove salts. This pool was analyzed using a Nanodrop 3000 to obtain its concentration and ensure high final purity measurements.

### 4. Pooled Target Library for Multiple Targets

We synthesized three pooled target sequences divided by GC content, all including a common adapter 1 “GCATGTCGTCAAGCAACAC” (SEQ ID NO: 353) flanking 60 nucleotides upstream that did not include the PAM, followed by 40 nucleotides downstream of the saCas9 PAM, including the PAM, and followed by a common adapter 2 “GCTGTGTATGTCCAAGTGTG” (SEQ ID NO: 354) (target region sequences are provided in SEQ ID NOs: 183-203, 226-247, and 270-291, which are the DNA target sequences plus a PAM.). We then PCR amplified this using an 189 nucleotide long forward Ultramer primer (IDT) of sequence 5′ AAGTATGTCGCAGAGCTGCAGCTGGAACGGCTGAAGAAAGATGGCGAGGTGAGAG GGTCAATTAATAGGTTCAAGACAAGCGACTACGTCAAAGAAGCCAAGCAGCTGCTG AAAGTGCAGAAGGCTTACCACCAGCTGGATCAGAGCTTCATCGATACTTATATCGAC CTGCATGTCGTCAAGCAACAC 3′ (SEQ ID NO: 355) and a 100 nucleotide long reverse Ultramer primer of sequence 5′ ACAACGCCCTGAATGACCTGAACAACCTGGTCATCACCAGGGATGAAAACGAGAAA CTGGAATACTATGAGAAGTTCCAGCACACTTGGACATACACAGC 3′ (SEQ ID NO: 356) to generate sequences that when cut could generate fragments suitable for gel shift extraction based purification.

5. saBLADE Gel Shift Activity Assay for Multiple Targets

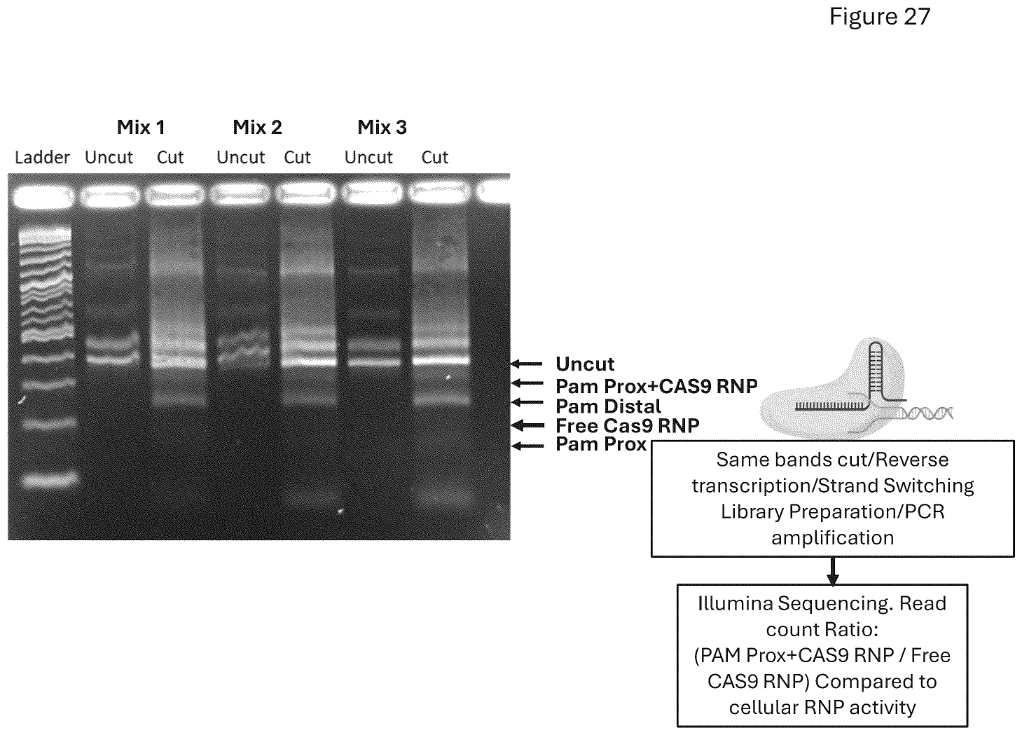

We incubated 200 ng of gRNA pool and 5 pmol of saCas9 protein for 10 minutes at 37 degrees. 1.35 ug of the corresponding target DNA library from method 5 was added and incubated for 10-minutes at 37 degrees. We ran this reaction with Purple Loading Dye lacking SDS (NEB Cat #B7025S) dye with 20 mM added MgCl2, in a pre-chilled unstained 4% Agarose gel at 4 degrees for 30 minutes at 30 Volts to avoid denaturing conditions. We then incubated the gel in Sybr gold 20× in 1×TAE, for 5 minutes prior to imaging. See FIG. 27. The PAM proximal band gel shifted by Cas9˜350 bp equivalent, and the free RNP (˜220 bp equivalent), were extracted, crushed and soaked in TE buffer overnight to allow for diffusion of complexes outside the gel. The samples were then treated with DNAse for 30 minutes, followed by Proteinase K prior to phenol chloroform extraction and ethanol precipitation. We proceeded to reverse transcription using the universal reverse transcription primer 5′AAAATCTCGCCAACAAGTTGACG 3′ (SEQ ID NO: 136) and MMLV reverse transcriptase, which adds non templated nucleotides after the RNA template 5′ end, allowing for template switching oligos produce full length cDNAs. We used locked nucleic acid oligonucleotide to act as a strand switching oligonucleotide. For strand switching, we used the Takara SMARTer small RNA Illumina library preparation kit (Cat 635029). We proceeded to send both gel extractions for high throughput paired-end next generation sequencing. See FIG. 27.

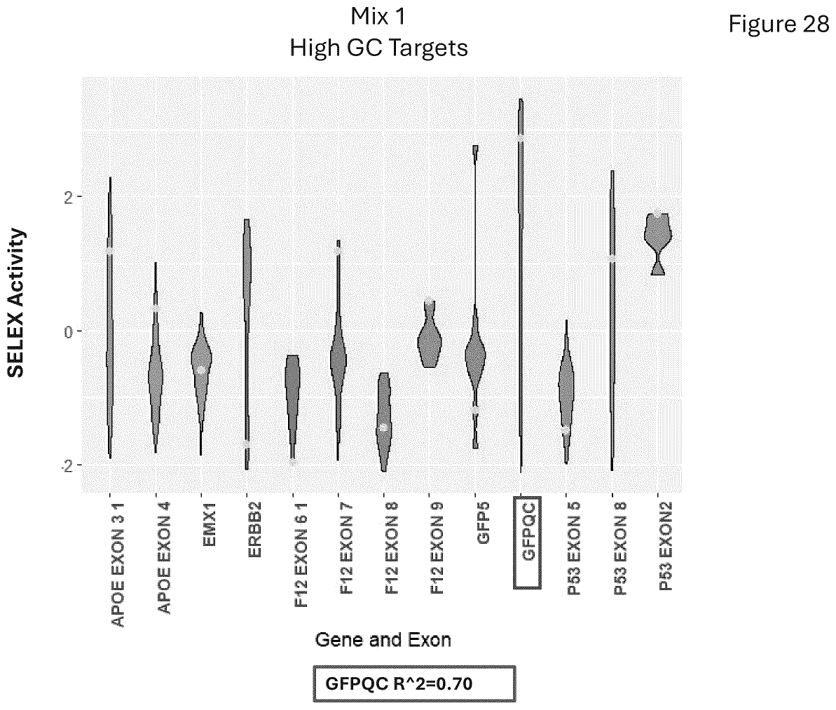

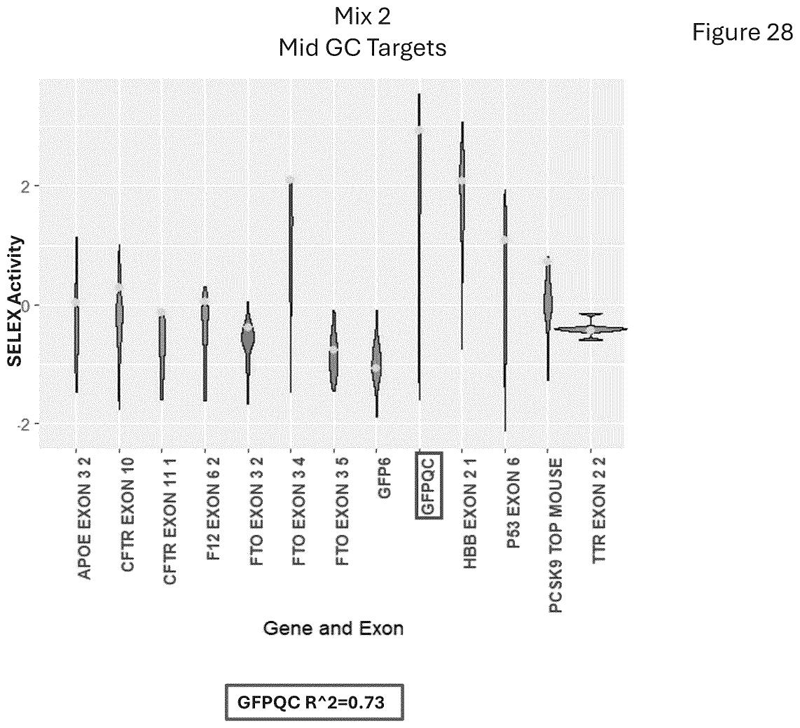

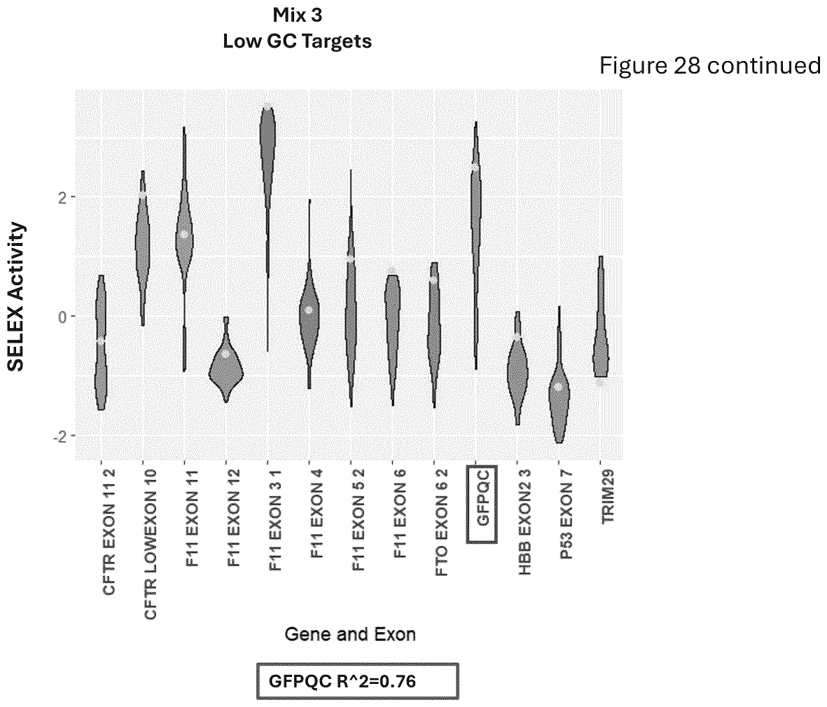

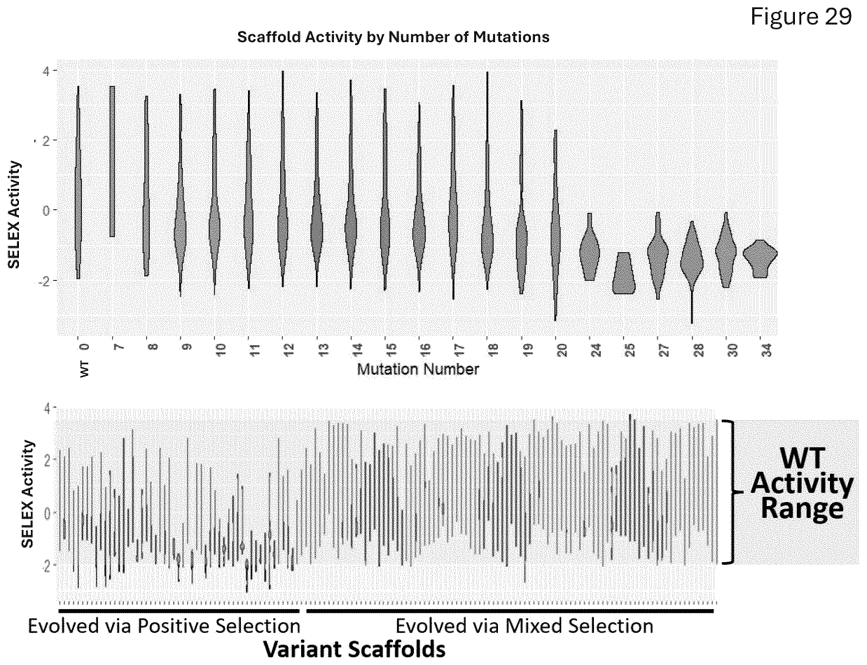

We carried out standard pre-processing including low quality score filtering, read ends trimming, forward and reverse read alignment and paired read joining. We calculated the Log Fold Change (LFC) between the Pam Prox cut band and the free RNP cut band. We plotted this ratio against our existing in cell activity data for GFP (see SEQ ID NOs: 292-345, these sequences are composed of GFP target “gCCGGTGGTGCAGATGAACTT” (SEQ ID NO: 225) followed by a gRNA scaffold as listed in SEQ ID NOs: 1-134, also see FIGS. 28 and 29).

This activity assay can rapidly inform the activity of variants obtained by saBLADE-SELEX in high throughput, given our assay correlations to in cell activity, additionally produced band measurements using next generation sequencing have a strong potential as high throughput characterization parameters for asymmetric target release gene editors.

### Example 3: Prime Editing Selection Methods

### Prime Editing Guide RNA Library Generation

Libraries were generated by annealing 1.5 nmol of synthesized single-stranded template library (5′-AAAAGAGCACGAGATGGCAGANNNNNNNNNNNNNNNNNNNNNNNNNNNNNNNNN NNNNNNNNNNNNNNNNNNNNNNNNNNNNNNNNNNNNNNNNNNNTCACGTGCTCA GTCTGGGCC-3′) (SEQ ID NO: 346) to 1 nmol of the forward primer (GGTAATACGACTCACTATAGGGCCCAGACTGAGCACGTGA) (SEQ ID NO: 347) in 10 mM Tris-HCl, pH 8.0, with 10 mM MgCl2 at 95° C. for 5 minutes. Libraries and primers were purchased through Integrated DNA Technologies (IDT; Coralville, IA). The annealed oligonucleotides were extended to full length with Exo(−) Klenow (New England Biolabs (NEB)) according to the manufacturer's protocol, phenol chloroform extracted, and concentrated and desalted with Amicon 10 kDa Ultra-0.5 mL columns (Millipore-Sigma) using 10 mM Tris pH 7.5 with 0.1 mM EDTA for column washes. The DNA libraries were then transcribed in vitro using T7 RNA polymerase (NEB) following the manufacturer's protocol using 250 pmol of DNA and 2 mM NTP mix (ThermoFisher). Transcribed libraries were treated with DNAse I (NEB) and purified using 10% polyacrylamide gel electrophoresis (PAGE; 19:1 acrylamide:bis-acrylamide (Biorad) in 1×TBE with 7 M urea). Excised RNA was eluted overnight in TE (10 mM Tris-Cl, 1 mM EDTA, pH 8) and ethanol precipitated.

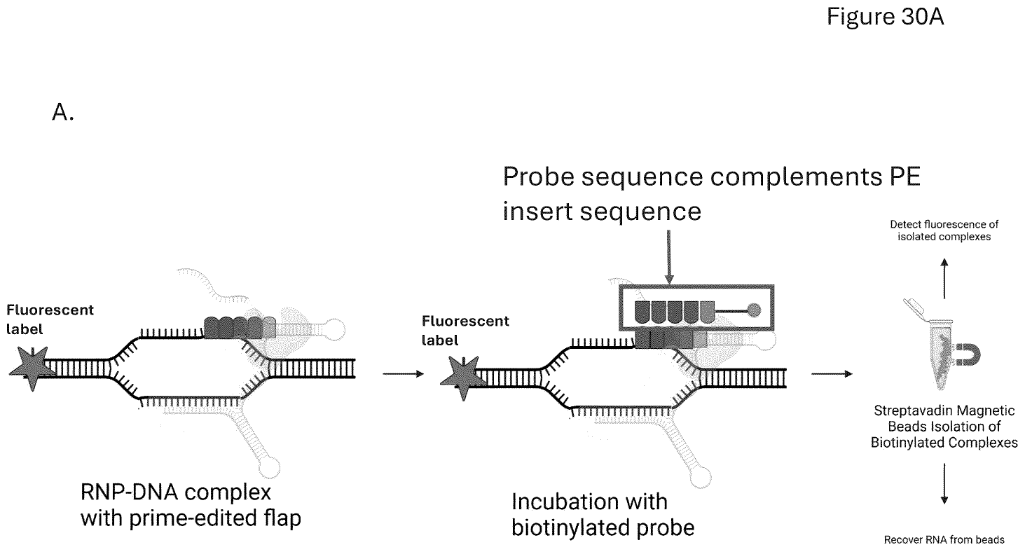

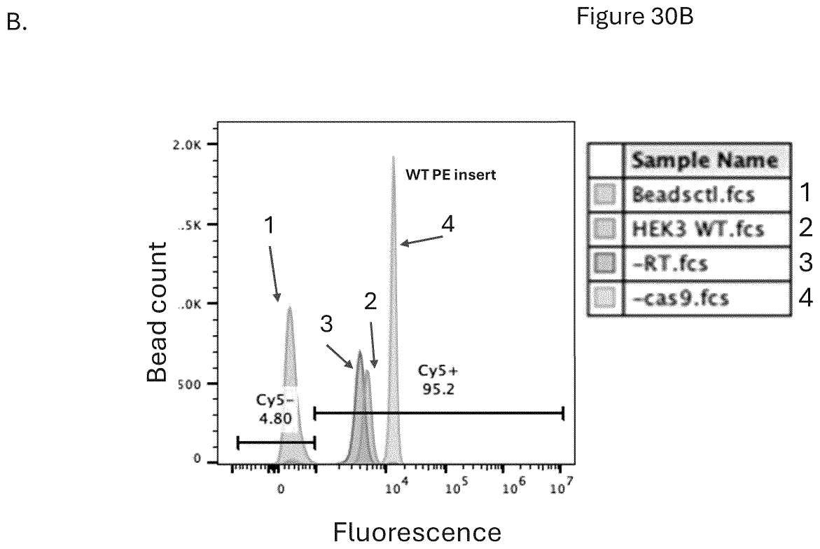

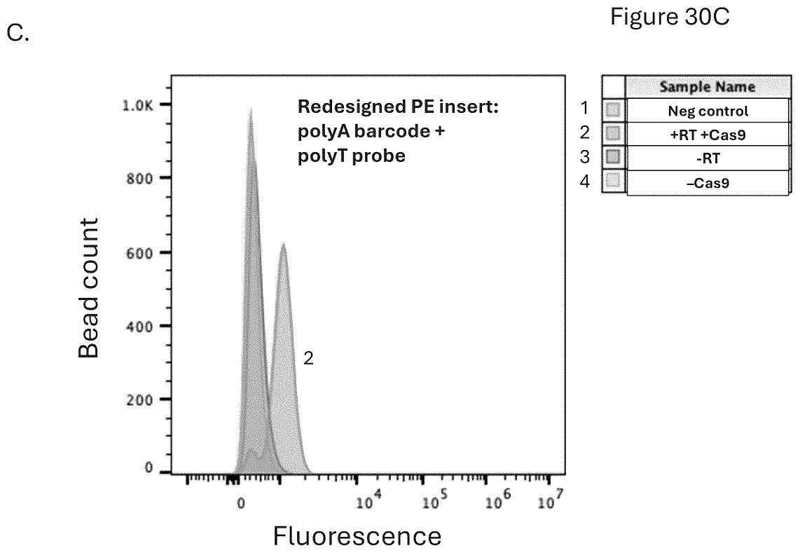

Selection Strategy 1: Biotinylated polyT Probe Matching Prime Edited polyA Barcode (FIG. 30)

During library generation, a modified single-stranded template library was used (5′-AAAAGAGCACGAAAAAAAAAAAGATGGCAGANNNNNNNNNNNNNNNNNNNNNNN NNNNNNNNNNNNNNNNNNNNNNNNNNNNNNNNNNNNNNNNNNNNNNNNNNNNNT CACGTGCTCAGTCTGGGCC-3′) (SEQ ID NO: 348) instead of the single-stranded template library described above, in which the bolded 10 As were inserted into the 3′ extended region of the pegRNA in order to facilitate inclusion of a polyA barcode in the prime edited sequence.

Each round of the selection was performed as follows: RNPs were formed by incubating transcribed prime editing guide RNA (pegRNA) libraries with H840A spCas9 nickase (IDT) at an equimolar ratio of 0.25 nmol in 1× cleavage buffer supplemented with dNTPs (20 mM HEPES-K, pH 7.5; 100 mM KCl; 5% glycerol; 0.2 mM EDTA, pH 8.0; 3 mM MgCl2; 0.5 mM dNTP mix; 5 mM DTT) for 10 minutes at room temperature. 40 pmol of target DNA substrate and 400 units of SuperScript III reverse transcriptase (ThermoFisher Scientific) were then added, and reactions were carried out at 37° C. for 1 hour. Unincorporated nucleotides were removed with an Amicon 10 kDa Ultra-0.5 mL column using 1× cleavage buffer without dNTPs for washes. One pmol of a biotinylated Oligo(dT) (Promega) was mixed with 2 μL of Streptavidin MyOne Cl Dynabeads (ThermoFisher) in NEB Buffer r3.1TE (NEB Buffer r3.1 supplemented with 0.005% Tween-20 (Sigma) and 10 mM EDTA) and incubated at 37° C. for 15 minutes with rotation. Probes that were bound to the magnetic beads were sequestered and washed three times in NEB Buffer r3.1TE. Probe-bead complexes were then mixed with the Amicon purified prime editing reactions, incubated at 37° C. for 15 minutes with rotation, and sequestered and washed three times in NEB Buffer r3.1TE. Target DNA was degraded with DNAse I treatment at 37° C. for 30 minutes, and bead-bound pegRNA variants were purified via phenol chloroform extraction followed by Amicon 10 kDa Ultra-0.5 mL column concentration. pegRNA libraries were then reverse transcribed using MMLV Reverse Transcriptase (Roche) and PCR amplified to generate a new DNA library. DNA libraries were transcribed and purified as described above, and this process was repeated for five rounds of selection.

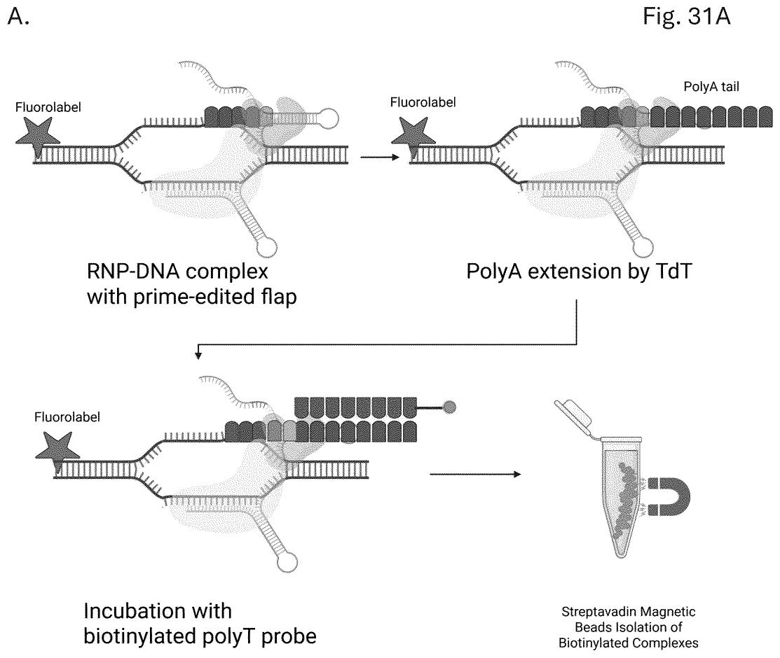

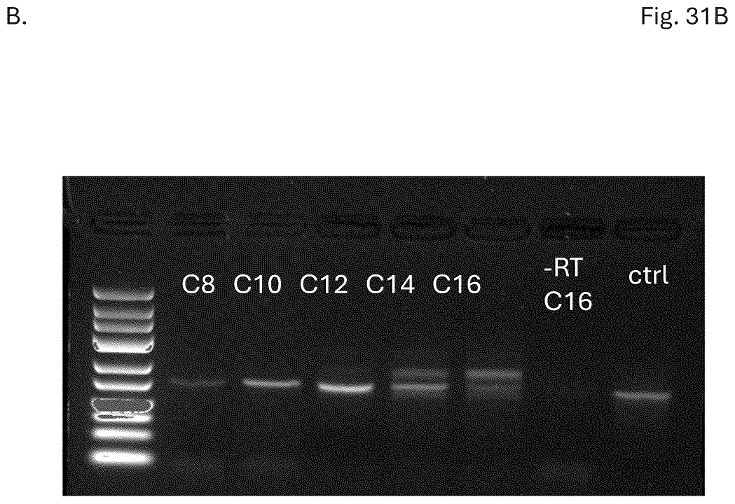

Selection Strategy 2: Biotinylated polyT Probe Matching Terminal Transferase (TdT) Generated polyA Barcode (FIG. 31)

Each round of the selection was performed as follows: RNPs were formed by incubating transcribed prime editing guide RNA (pegRNA) libraries with H840A spCas9 nickase (IDT) at an equimolar ratio of 0.25 nmol in 1× cleavage buffer supplemented with dNTPs (20 mM HEPES-K, pH 7.5; 100 mM KCl; 5% glycerol; 0.2 mM EDTA, pH 8.0; 3 mM MgCl2; 0.5 mM dNTP mix; 5 mM DTT) for 10 minutes at room temperature. 40 pmol of target DNA substrate and 400 units of SuperScript III reverse transcriptase (ThermoFisher Scientific) were then added, and reactions were carried out at 37° C. for 1 hour. Unincorporated nucleotides were removed with an Amicon 10 kDa Ultra-0.5 mL column using 1× cleavage buffer without dNTPs for washes. Subsequently, polyA barcodes were generated on the 3′ ends of edited DNA by adding 100 units of recombinant E. coli terminal deoxynucleotidyl transferase (TdT; Millipore-Sigma), 5× reaction buffer to a final concentration of 1×, 5 mM CoCl2, and 1 mM dATP to the reaction and incubating at 37° C. for 30 minutes. One pmol of a biotinylated Oligo(dT) (Promega) was mixed with 2 L of Streptavidin MyOne C1 Dynabeads (ThermoFisher) in NEB Buffer r3.1TE (NEB Buffer r3.1 supplemented with 0.005% Tween-20 (Sigma) and 10 mM EDTA) and incubated at 37° C. for 15 minutes with rotation. Probes that were bound to the magnetic beads were sequestered and washed three times in NEB Buffer r3.1TE. Probe-bead complexes were then mixed with the TdT-barcoded prime editing reactions, incubated at 37° C. for 15 minutes with rotation, and sequestered and washed three times in NEB Buffer r3.1TE. Target DNA was degraded with DNAse I treatment at 37° C. for 30 minutes, and bead-bound pegRNA variants were purified via phenol chloroform extraction followed by Amicon 10 kDa Ultra-0.5 mL column concentration. pegRNA libraries were then reverse transcribed using MMLV Reverse Transcriptase (Roche) and PCR amplified to generate a new DNA library. DNA libraries were transcribed and purified as described above, and this process was repeated for five rounds of selection.

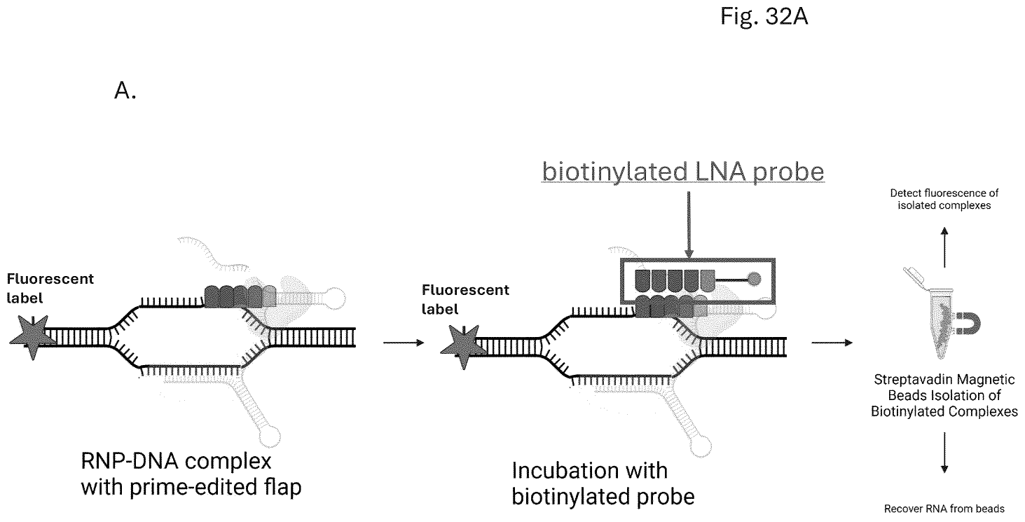

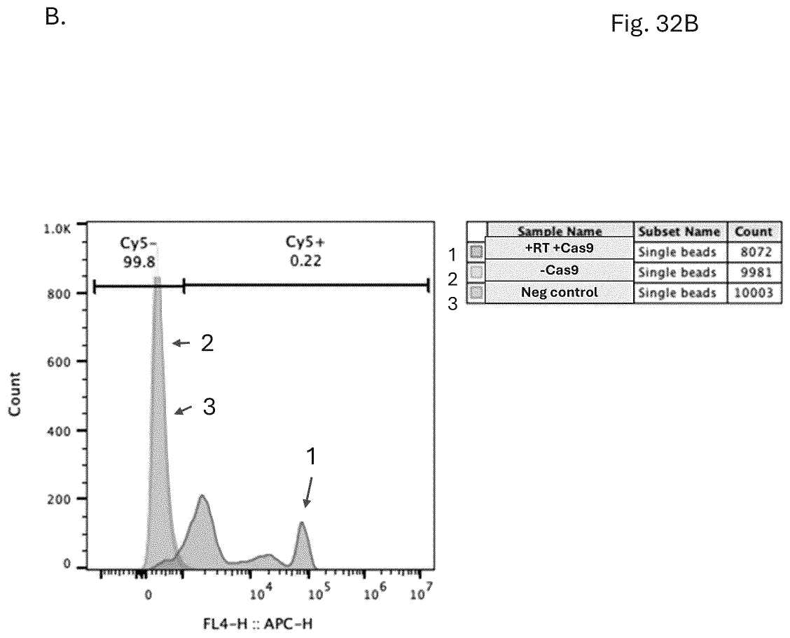

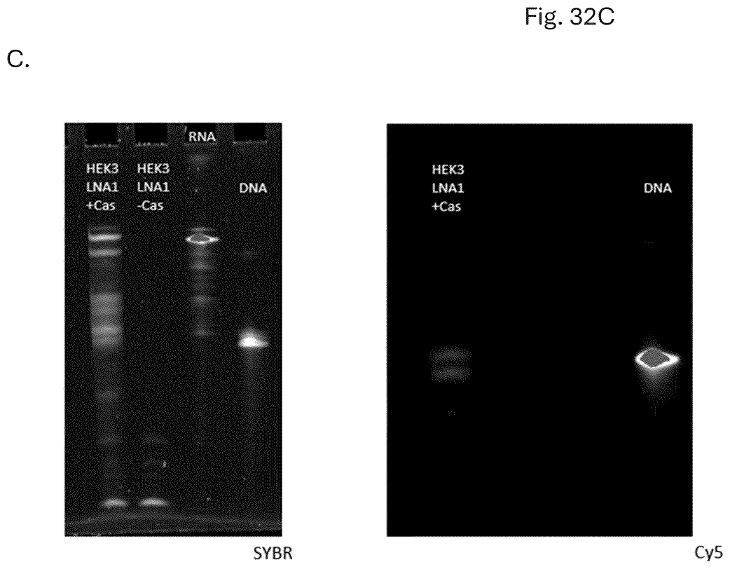

### Selection Strategy 3: Biotinylated Locked Nucleic Acid (LNA) Probe Matching Prime Edited Sequence (FIG. 32)

Each round of the selection was performed as follows: RNPs were formed by incubating transcribed prime editing guide RNA (pegRNA) libraries with H840A spCas9 nickase (IDT) at an equimolar ratio of 0.25 nmol in 1× cleavage buffer supplemented with dNTPs (20 mM HEPES-K, pH 7.5; 100 mM KCl; 5% glycerol; 0.2 mM EDTA, pH 8.0; 3 mM MgCl2; 0.5 mM dNTP mix; 5 mM DTT) for 10 minutes at room temperature. 40 pmol of target DNA substrate and 400 units of SuperScript III reverse transcriptase (ThermoFisher Scientific) were then added, and reactions were carried out at 37° C. for 1 hour. Unincorporated nucleotides were removed with an Amicon 10 kDa Ultra-0.5 mL column using 1× cleavage buffer without dNTPs for washes. One pmol of a biotinylated single-stranded locked nucleic acid (LNA) oligonucleotide (5′-TG+CCAT+C+T+CGTG+CT-3′ (SEQ ID NO: 349), where bases with a + in front of them designate LNA bases) purchased through IDT was mixed with 2 μL of Streptavidin MyOne C1 Dynabeads (ThermoFisher) in NEB Buffer r3.1TE (NEB Buffer r3.1 supplemented with 0.005% Tween-20 (Sigma) and 10 mM EDTA) and incubated at 37° C. for 15 minutes with rotation. LNA probes that were bound to the magnetic beads were sequestered and washed three times in NEB Buffer r3.1TE. Probe-bead complexes were then mixed with the Amicon purified prime editing reactions, incubated at 37° C. for 15 minutes with rotation, and sequestered and washed three times in NEB Buffer r3.1TE. Target DNA was degraded with DNAse I treatment at 37° C. for 30 minutes, and bead-bound pegRNA variants were purified via phenol chloroform extraction followed by Amicon 10 kDa Ultra-0.5 mL column concentration. pegRNA libraries were then reverse transcribed using MMLV Reverse Transcriptase (Roche) and PCR amplified to generate a new DNA library. DNA libraries were transcribed and purified as described above, and this process was repeated for five rounds of selection.

1. A composition comprising a gRNA scaffold comprising a sequence selected from the group consisting of SEQ ID NOs: 1-134 or a sequence having at least 98% identity to at least one of SEQ ID NO: 1-134.

2. The composition of claim 1, further comprising a gRNA target region.

3. The composition of claim 2, wherein the gRNA target region is about 20 nucleotides in length and is proximate to a protospacer adjacent motif (PAM).

4. (canceled)

5. The composition of claim 1, further comprising a Cas protein capable of forming a ribonucleoprotein complex with the gRNA scaffold.

6. The composition of claim 5, wherein the Cas protein is a Staphylococcus aureus Cas9 protein or a variant thereof capable of forming a ribonucleoprotein complex with at least one of the gRNA scaffolds in the composition.

7. (canceled)

8. A construct comprising the composition of claim 1, wherein the construct comprises a first polynucleotide encoding a Cas9 protein operably linked to a promoter to allow expression of the Cas9 protein and a second polynucleotide operably linked to a promoter, wherein the second polynucleotide comprises a sequence capable of encoding the gRNA scaffold.

9. (canceled)

10. (canceled)

11. A method of using the composition of claim 1 for gene editing in a cell comprising introducing the composition into the cell to allow genetic editing of the cell producing an edited cell and selecting the edited cell comprising an edited target.

12. A method of generating a guide nucleic acid (gRNA) capable of binding a Cas protein, the method comprising:
a. generating a RNP complex pool by combining a Cas protein with a gRNA having a conserved target region and a randomized scaffold region;
b. introducing a target DNA bound to or capable of binding to an affinity reagent, wherein the DNA comprises a PAM site and sequence complementary to the conserved target region of the gRNA,
c. mixing the RNP complex with the affinity reagent and target DNA to generate an RNP-DNA-affinity reagent mixture,
d. separating an RNP-DNA-affinity reagent complex from the DNA-affinity reagent mixture, and
e. harvesting the gRNA from the RNP-DNA-affinity reagent complex.

13. The method of claim 12, wherein the gRNA is capable of directing the Cas protein to perform site-specific cleavage of a targeted double-stranded DNA proximate to a PAM site.

14. The method of claim 12, further comprising at least one of:
i. sequencing the gRNA,
ii. reverse transcribing and cloning the gRNA into a vector;
iii. reverse transcribing and amplifying the gRNA.

15. (canceled)

16. (canceled)

17. The method of claim 12, further comprising repeating steps (a)-(e) multiple rounds.

18. The method of claim 12, wherein the target DNA is labeled on the 5′ end proximal to the PAM site with the affinity reagent or wherein the target DNA is labeled on the 5′ end distal to the PAM site with the affinity reagent.

19.-21. (canceled)

22. A method of enriching recovery of edited sequences after prime editing comprising:
(a) generating a ribonucleoprotein (RNP)-DNA complex by contacting at least one prime editing guide RNA (pegRNA) with a target DNA and a prime editing protein complex comprising a Cas9 nickase and a reverse transcriptase (RT), wherein the pegRNA comprises a 5′ region and a 3′ region complementary to the target DNA and an intended edited region to be incorporated into the target DNA the reverse transcript;
(b) incubating the RNP-DNA complex to allow the Cas nickase to generate a nick in the target DNA and the RT to incorporate the complement of the intended edit into the nicked strand of the target DNA to generate a single-strand DNA incorporated edit;
(c) introducing a nucleic acid probe sequence complementary to the single-strand DNA incorporated edit to bind to the edited DNA, wherein the probe is labeled or is capable of binding to a label; and
(d) separating the edited DNA by selecting for the label and thus enriching for recovery of edited sequences.

23. The method of claim 22, wherein the probe further comprises a homopolynucleotide.

24. The method of claim 23, wherein the homopolynucleotide is a polyadenine or polycytosine.

25.-28. (canceled)

29. The method of claim 22, wherein the probe comprises at least one locked nucleic acid.

30. The method of claim 22, wherein the pegRNA is a pool of pegRNA comprising at least 10 pegRNAs designed to edit the same target DNA and including the intended edited region.

31. (canceled)

32. The method of claim 22, wherein the pegRNA is capable of binding to a modified or engineered Cas protein.

33. The method of claim 22, wherein the Cas9 protein or modified Cas9 protein is a Streptococcus pyogenes or Staphylococcus aureus Cas9 protein.

34. (canceled)

35. The method of claim 22, further comprising recovering the pegRNA from the separated edited DNA.


SEQUENCE LISTING (415 sequences)

SEQ ID NO: 1 - RNA, 80 nt, synthetic construct
  modified_base 2..5: mod_base = OTHER; note = thymine
  modified_base 11: mod_base = OTHER; note = thymine
  modified_base 13: mod_base = OTHER; note = thymine
  modified_base 18: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 46: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttaggactctggaataagaacctcctaaaacaagacaagatgtcgcgttcatcacgtcaacttgttggcgagatttt

SEQ ID NO: 2 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 41..42: mod_base = OTHER; note = thymine
  modified_base 44: mod_base = OTHER; note = thymine
  modified_base 47: mod_base = OTHER; note = thymine
  modified_base 49: mod_base = OTHER; note = thymine
  modified_base 51..52: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttcgtactctggagacggaatctacgaaaacaaagcattatgctgtgttcagcccgtcaacttgttggcgagatttt

SEQ ID NO: 3 - RNA, 80 nt, synthetic construct
  modified_base 2..4: mod_base = OTHER; note = thymine
  modified_base 8: mod_base = OTHER; note = thymine
  modified_base 11: mod_base = OTHER; note = thymine
  modified_base 26: mod_base = OTHER; note = thymine
  modified_base 28..29: mod_base = OTHER; note = thymine
  modified_base 44: mod_base = OTHER; note = thymine
  modified_base 46: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtttgagtactcgggccaccgaacctattcaaacaagacaaaatgtcgcgttcatcacgtcaacttgttggcgagatttt

SEQ ID NO: 4 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 20: mod_base = OTHER; note = thymine
  modified_base 24: mod_base = OTHER; note = thymine
  modified_base 26: mod_base = OTHER; note = thymine
  modified_base 44: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttgtgatacccagccagctggatctaccacaacaacgcaaaatgcggggttcgtcacgtcaacttgttggcgagatttt

SEQ ID NO: 5 - RNA, 80 nt, synthetic construct
  modified_base 2: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 16: mod_base = OTHER; note = thymine
  modified_base 18: mod_base = OTHER; note = thymine
  modified_base 26: mod_base = OTHER; note = thymine
  modified_base 28..29: mod_base = OTHER; note = thymine
  modified_base 40: mod_base = OTHER; note = thymine
  modified_base 44: mod_base = OTHER; note = thymine
  modified_base 46: mod_base = OTHER; note = thymine
  modified_base 49: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtcgtagtactctcgtgtgagaacctattacgacaagactacgtgtcgtgttcatcacgtcaacttgttggcgagatttt

SEQ ID NO: 6 - RNA, 80 nt, synthetic construct
  modified_base 2..3: mod_base = OTHER; note = thymine
  modified_base 5: mod_base = OTHER; note = thymine
  modified_base 7..8: mod_base = OTHER; note = thymine
  modified_base 13: mod_base = OTHER; note = thymine
  modified_base 15: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 41: mod_base = OTHER; note = thymine
  modified_base 44: mod_base = OTHER; note = thymine
  modified_base 47: mod_base = OTHER; note = thymine
  modified_base 49: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttctattaccctgtaaagcggatctagtagaacaaagcataatgctgtagtcatgacgtcaacttgttggcgagatttt

SEQ ID NO: 7 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 13..14: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 31: mod_base = OTHER; note = thymine
  modified_base 37: mod_base = OTHER; note = thymine
  modified_base 40: mod_base = OTHER; note = thymine
  modified_base 44: mod_base = OTHER; note = thymine
  modified_base 49: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttatactactcttgaaagggaatctagtataacagtgctgaatgcagtgttcatgccgtcaacttgttggcgagatttt

SEQ ID NO: 8 - RNA, 80 nt, synthetic construct
  modified_base 2..4: mod_base = OTHER; note = thymine
  modified_base 8: mod_base = OTHER; note = thymine
  modified_base 11: mod_base = OTHER; note = thymine
  modified_base 15: mod_base = OTHER; note = thymine
  modified_base 18..19: mod_base = OTHER; note = thymine
  modified_base 26: mod_base = OTHER; note = thymine
  modified_base 29: mod_base = OTHER; note = thymine
  modified_base 40: mod_base = OTHER; note = thymine
  modified_base 42: mod_base = OTHER; note = thymine
  modified_base 44: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtttcagtactccgtagttggaacctactgaaacaaggctatatgccggggtcataacgtcaacttgttggcgagatttt

SEQ ID NO: 9 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 18: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 40: mod_base = OTHER; note = thymine
  modified_base 44: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagtactgtggactcacaatctactaaaacaacgctgaatgcggcattcatcccgtcaacttgttggcgagatttt

SEQ ID NO: 10 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 47: mod_base = OTHER; note = thymine
  modified_base 49: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttgtagtactctgcaaacagaatctactacaacagagcaaaatgctgtggacatcacgtcaacttgttggcgagatttt

SEQ ID NO: 11 - RNA, 80 nt, synthetic construct
  modified_base 2..4: mod_base = OTHER; note = thymine
  modified_base 8: mod_base = OTHER; note = thymine
  modified_base 11: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 46: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtttcagtactcacggaacagaatctactgaaacaagacaagatgtcgcattcatcacgtcaacttgttggcgagatttt

SEQ ID NO: 12 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 14: mod_base = OTHER; note = thymine
  modified_base 24: mod_base = OTHER; note = thymine
  modified_base 28..29: mod_base = OTHER; note = thymine
  modified_base 44: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttgtagtgctcgtgcaaccgaatccattacaacaacgcaaaatgcggcggtcatcacgtcaacttgttggcgagatttt

SEQ ID NO: 13 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 14: mod_base = OTHER; note = thymine
  modified_base 19: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 40: mod_base = OTHER; note = thymine
  modified_base 44: mod_base = OTHER; note = thymine
  modified_base 49: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttctagtactcgtggaatggaatctactagaacaacgctgaatgcggtgctcatcccgtcaacttgttggcgagatttt

SEQ ID NO: 14 - RNA, 80 nt, synthetic construct
  modified_base 2..4: mod_base = OTHER; note = thymine
  modified_base 8: mod_base = OTHER; note = thymine
  modified_base 11: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 37: mod_base = OTHER; note = thymine
  modified_base 44: mod_base = OTHER; note = thymine
  modified_base 49: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtttcagtactcgagccaccgaatctactgaaacagtgcgaaatgcagtcttcatcacgtcaacttgttggcgagatttt

SEQ ID NO: 15 - RNA, 80 nt, synthetic construct
  modified_base 2..5: mod_base = OTHER; note = thymine
  modified_base 8: mod_base = OTHER; note = thymine
  modified_base 13..15: mod_base = OTHER; note = thymine
  modified_base 22: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 37: mod_base = OTHER; note = thymine
  modified_base 42: mod_base = OTHER; note = thymine
  modified_base 44: mod_base = OTHER; note = thymine
  modified_base 49..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagtacactttaacgagtatctactaaaacaatgcggtatgcagttttcatcacgtcaacttgttggcgagatttt

SEQ ID NO: 16 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 15: mod_base = OTHER; note = thymine
  modified_base 26: mod_base = OTHER; note = thymine
  modified_base 29: mod_base = OTHER; note = thymine
  modified_base 37: mod_base = OTHER; note = thymine
  modified_base 44: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttactactctgtgaacggaacctagtaaaacaatgcacgatgcagcgttcatcccgtcaacttgttggcgagatttt

SEQ ID NO: 17 - RNA, 80 nt, synthetic construct
  modified_base 3..4: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 17: mod_base = OTHER; note = thymine
  modified_base 20..21: mod_base = OTHER; note = thymine
  modified_base 23..24: mod_base = OTHER; note = thymine
  modified_base 29: mod_base = OTHER; note = thymine
  modified_base 32..33: mod_base = OTHER; note = thymine
  modified_base 39: mod_base = OTHER; note = thymine
  modified_base 41: mod_base = OTHER; note = thymine
  modified_base 44: mod_base = OTHER; note = thymine
  modified_base 47: mod_base = OTHER; note = thymine
  modified_base 51: mod_base = OTHER; note = thymine
  modified_base 53..54: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
cgttgggtactctcaatcgttcttccactggttcaacgtctagtcctgggtcttgcccgtcaacttgttggcgagatttt

SEQ ID NO: 18 - RNA, 80 nt, synthetic construct
  modified_base 3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 26: mod_base = OTHER; note = thymine
  modified_base 29: mod_base = OTHER; note = thymine
  modified_base 41: mod_base = OTHER; note = thymine
  modified_base 44: mod_base = OTHER; note = thymine
  modified_base 47: mod_base = OTHER; note = thymine
  modified_base 49: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gctgtagtactctggaaacagaacctgctacagcaaagcatgatgctgtgttcatgccgtcaacttgttggcgagatttt

SEQ ID NO: 19 - RNA, 80 nt, synthetic construct
  modified_base 4..5: mod_base = OTHER; note = thymine
  modified_base 13: mod_base = OTHER; note = thymine
  modified_base 17: mod_base = OTHER; note = thymine
  modified_base 24: mod_base = OTHER; note = thymine
  modified_base 30..31: mod_base = OTHER; note = thymine
  modified_base 38: mod_base = OTHER; note = thymine
  modified_base 44: mod_base = OTHER; note = thymine
  modified_base 51..53: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
ggcttgggacggtggatgcggactccccgttaaggagtggaactggcgactttgcaccgtcaacttgttggcgagatttt

SEQ ID NO: 20 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 16: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 40: mod_base = OTHER; note = thymine
  modified_base 44: mod_base = OTHER; note = thymine
  modified_base 49: mod_base = OTHER; note = thymine
  modified_base 53: mod_base = OTHER; note = thymine
  modified_base 55..56: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagtaatgcggtcccgcaatctactaaaacaacgctgaatgcggtgggtattccgtcaacttgttggcgagatttt

SEQ ID NO: 21 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 24: mod_base = OTHER; note = thymine
  modified_base 29: mod_base = OTHER; note = thymine
  modified_base 40..44: mod_base = OTHER; note = thymine
  modified_base 49: mod_base = OTHER; note = thymine
  modified_base 55..56: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagtgctcgggcaaccgaatccactaaaacaacgctttttgcggtgggcattacgtcaacttgttggcgagatttt

SEQ ID NO: 22 - RNA, 80 nt, synthetic construct
  modified_base 2..6: mod_base = OTHER; note = thymine
  modified_base 8: mod_base = OTHER; note = thymine
  modified_base 24: mod_base = OTHER; note = thymine
  modified_base 26: mod_base = OTHER; note = thymine
  modified_base 39: mod_base = OTHER; note = thymine
  modified_base 44: mod_base = OTHER; note = thymine
  modified_base 51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
ctttttgtaaccgggaaaaaggatctaccaaaagaacgtgacatgcggggtgcatcccgtcaacttgttggcgagatttt

SEQ ID NO: 23 - RNA, 80 nt, synthetic construct
  modified_base 2: mod_base = OTHER; note = thymine
  modified_base 4: mod_base = OTHER; note = thymine
  modified_base 6..10: mod_base = OTHER; note = thymine
  modified_base 13: mod_base = OTHER; note = thymine
  modified_base 17: mod_base = OTHER; note = thymine
  modified_base 26: mod_base = OTHER; note = thymine
  modified_base 29: mod_base = OTHER; note = thymine
  modified_base 35: mod_base = OTHER; note = thymine
  modified_base 48..49: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtgtctttttgctaaatcccgccactagtaaaactgagcacagggcattggtgataccgtcaacttgttggcgagatttt

SEQ ID NO: 24 - RNA, 80 nt, synthetic construct
  modified_base 2..4: mod_base = OTHER; note = thymine
  modified_base 15: mod_base = OTHER; note = thymine
  modified_base 24..27: mod_base = OTHER; note = thymine
  modified_base 29: mod_base = OTHER; note = thymine
  modified_base 44: mod_base = OTHER; note = thymine
  modified_base 49: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtttcagaacgccgtcagcggagttttctgaaacaaggcgaaatgccgtgggcatcccgtcaacttgttggcgagatttt

SEQ ID NO: 25 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 40: mod_base = OTHER; note = thymine
  modified_base 42: mod_base = OTHER; note = thymine
  modified_base 44: mod_base = OTHER; note = thymine
  modified_base 49: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagtactctagaaaaagagtctgctaaaacgaggctatatgccgtgggcatcccgtcaacttgttggcgagatttt

SEQ ID NO: 26 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 26: mod_base = OTHER; note = thymine
  modified_base 29: mod_base = OTHER; note = thymine
  modified_base 40..42: mod_base = OTHER; note = thymine
  modified_base 44: mod_base = OTHER; note = thymine
  modified_base 49: mod_base = OTHER; note = thymine
  modified_base 52..53: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagtactctggaaacaggacctactaaaacaacgctttatgcggtggttatgacgtcaacttgttggcgagatttt

SEQ ID NO: 27 - RNA, 80 nt, synthetic construct
  modified_base 2..5: mod_base = OTHER; note = thymine
  modified_base 8..9: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 31: mod_base = OTHER; note = thymine
  modified_base 34: mod_base = OTHER; note = thymine
  modified_base 36..40: mod_base = OTHER; note = thymine
  modified_base 44..46: mod_base = OTHER; note = thymine
  modified_base 49: mod_base = OTHER; note = thymine
  modified_base 52..53: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttcgttctctggaaacagaatcaacgataatatttttggatttcgtggttaccccgtcaacttgttggcgagatttt

SEQ ID NO: 28 - RNA, 80 nt, synthetic construct
  modified_base 2..3: mod_base = OTHER; note = thymine
  modified_base 7: mod_base = OTHER; note = thymine
  modified_base 11: mod_base = OTHER; note = thymine
  modified_base 23: mod_base = OTHER; note = thymine
  modified_base 26: mod_base = OTHER; note = thymine
  modified_base 29: mod_base = OTHER; note = thymine
  modified_base 35: mod_base = OTHER; note = thymine
  modified_base 40..42: mod_base = OTHER; note = thymine
  modified_base 44..45: mod_base = OTHER; note = thymine
  modified_base 49: mod_base = OTHER; note = thymine
  modified_base 53: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
attgcatggctaaggggacagatcgtcctgaaactagggtttgttcggtgactcccacgtcaacttgttggcgagatttt

SEQ ID NO: 29 - RNA, 80 nt, synthetic construct
  modified_base 2..5: mod_base = OTHER; note = thymine
  modified_base 13: mod_base = OTHER; note = thymine
  modified_base 20: mod_base = OTHER; note = thymine
  modified_base 24: mod_base = OTHER; note = thymine
  modified_base 26..27: mod_base = OTHER; note = thymine
  modified_base 29: mod_base = OTHER; note = thymine
  modified_base 34: mod_base = OTHER; note = thymine
  modified_base 44: mod_base = OTHER; note = thymine
  modified_base 46: mod_base = OTHER; note = thymine
  modified_base 51..53: mod_base = OTHER; note = thymine
  modified_base 56: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
attttaggaacctgggccatggatcttctaaaataagacaggatgtcgcgtttagtccgtcaacttgttggcgagatttt

SEQ ID NO: 30 - RNA, 80 nt, synthetic construct
  modified_base 3..5: mod_base = OTHER; note = thymine
  modified_base 8: mod_base = OTHER; note = thymine
  modified_base 11..13: mod_base = OTHER; note = thymine
  modified_base 19: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 47: mod_base = OTHER; note = thymine
  modified_base 49: mod_base = OTHER; note = thymine
  modified_base 51: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gctttagtactttagaaatagaacctactaaagcagagcagaatgctgtgtgcaagacgtcaacttgttggcgagatttt

SEQ ID NO: 31 - RNA, 80 nt, synthetic construct
  modified_base 2..3: mod_base = OTHER; note = thymine
  modified_base 8: mod_base = OTHER; note = thymine
  modified_base 13: mod_base = OTHER; note = thymine
  modified_base 18: mod_base = OTHER; note = thymine
  modified_base 26..27: mod_base = OTHER; note = thymine
  modified_base 29: mod_base = OTHER; note = thymine
  modified_base 44..46: mod_base = OTHER; note = thymine
  modified_base 48: mod_base = OTHER; note = thymine
  modified_base 51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttagagtacactggactaagccccttgtcgaagacgcgagactttctggtgcgtcccgtcaacttgttggcgagatttt

SEQ ID NO: 32 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 41: mod_base = OTHER; note = thymine
  modified_base 44: mod_base = OTHER; note = thymine
  modified_base 49: mod_base = OTHER; note = thymine
  modified_base 52..53: mod_base = OTHER; note = thymine
  modified_base 56: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttgtactactctgcaaaaggaatctagtgcaacaaggcataatgccgtggttagtacgtcaacttgttggcgagatttt

SEQ ID NO: 33 - RNA, 80 nt, synthetic construct
  modified_base 2..4: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 29..30: mod_base = OTHER; note = thymine
  modified_base 44: mod_base = OTHER; note = thymine
  modified_base 49: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtttacgtactctgaagacagaatctacttaaacaaggccacatgccgtagtcataacgtcaacttgttggcgagatttt

SEQ ID NO: 34 - RNA, 80 nt, synthetic construct
  modified_base 2..4: mod_base = OTHER; note = thymine
  modified_base 8: mod_base = OTHER; note = thymine
  modified_base 23..25: mod_base = OTHER; note = thymine
  modified_base 27: mod_base = OTHER; note = thymine
  modified_base 32..33: mod_base = OTHER; note = thymine
  modified_base 40: mod_base = OTHER; note = thymine
  modified_base 44: mod_base = OTHER; note = thymine
  modified_base 48: mod_base = OTHER; note = thymine
  modified_base 51..52: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtttgagtgcgagggaaccggatttgtcaaattcgaggctgcctgcatggttagggccgtcaacttgttggcgagatttt

SEQ ID NO: 35 - RNA, 80 nt, synthetic construct
  modified_base 2..5: mod_base = OTHER; note = thymine
  modified_base 8..9: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 29: mod_base = OTHER; note = thymine
  modified_base 44: mod_base = OTHER; note = thymine
  modified_base 52: mod_base = OTHER; note = thymine
  modified_base 55: mod_base = OTHER; note = thymine
  modified_base 57: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagttctctgggaccagaatcagctaaaacaaggcaaaatgccggggtgatgtcgtcaacttgttggcgagatttt

SEQ ID NO: 36 - RNA, 80 nt, synthetic construct
  modified_base 2..5: mod_base = OTHER; note = thymine
  modified_base 8: mod_base = OTHER; note = thymine
  modified_base 10..11: mod_base = OTHER; note = thymine
  modified_base 13: mod_base = OTHER; note = thymine
  modified_base 16: mod_base = OTHER; note = thymine
  modified_base 19: mod_base = OTHER; note = thymine
  modified_base 24: mod_base = OTHER; note = thymine
  modified_base 40: mod_base = OTHER; note = thymine
  modified_base 43..44: mod_base = OTHER; note = thymine
  modified_base 49: mod_base = OTHER; note = thymine
  modified_base 52..53: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttcgtgttctggtagtagaatccacgaaaacaacgctagttgcggtggttatcacgtcaacttgttggcgagatttt

SEQ ID NO: 37 - RNA, 80 nt, synthetic construct
  modified_base 1: mod_base = OTHER; note = thymine
  modified_base 4..5: mod_base = OTHER; note = thymine
  modified_base 10: mod_base = OTHER; note = thymine
  modified_base 13: mod_base = OTHER; note = thymine
  modified_base 19: mod_base = OTHER; note = thymine
  modified_base 24: mod_base = OTHER; note = thymine
  modified_base 29: mod_base = OTHER; note = thymine
  modified_base 32: mod_base = OTHER; note = thymine
  modified_base 37: mod_base = OTHER; note = thymine
  modified_base 42..44: mod_base = OTHER; note = thymine
  modified_base 46: mod_base = OTHER; note = thymine
  modified_base 48..49: mod_base = OTHER; note = thymine
  modified_base 52: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
tgcttgggatgctcggcatagaatcgactcgtaggatgcgatttgtattcctgcccgcgtcaacttgttggcgagatttt

SEQ ID NO: 38 - RNA, 80 nt, synthetic construct
  modified_base 1: mod_base = OTHER; note = thymine
  modified_base 3..4: mod_base = OTHER; note = thymine
  modified_base 7..8: mod_base = OTHER; note = thymine
  modified_base 11: mod_base = OTHER; note = thymine
  modified_base 13: mod_base = OTHER; note = thymine
  modified_base 18: mod_base = OTHER; note = thymine
  modified_base 20: mod_base = OTHER; note = thymine
  modified_base 24: mod_base = OTHER; note = thymine
  modified_base 31: mod_base = OTHER; note = thymine
  modified_base 44: mod_base = OTHER; note = thymine
  modified_base 48..49: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
tgttgattcctctggaatatgcatcgagaatggcaaaccaaaatgcattcttcctgacgtcaacttgttggcgagatttt

SEQ ID NO: 39 - RNA, 80 nt, synthetic construct
  modified_base 2..4: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 26: mod_base = OTHER; note = thymine
  modified_base 28..29: mod_base = OTHER; note = thymine
  modified_base 34: mod_base = OTHER; note = thymine
  modified_base 40: mod_base = OTHER; note = thymine
  modified_base 44: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
atttcagtaatctggaaacagaagctgttgaaataaggctaaatgccgcgatgataacgtcaacttgttggcgagatttt

SEQ ID NO: 40 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13..14: mod_base = OTHER; note = thymine
  modified_base 17: mod_base = OTHER; note = thymine
  modified_base 20: mod_base = OTHER; note = thymine
  modified_base 24: mod_base = OTHER; note = thymine
  modified_base 26: mod_base = OTHER; note = thymine
  modified_base 28: mod_base = OTHER; note = thymine
  modified_base 34: mod_base = OTHER; note = thymine
  modified_base 44: mod_base = OTHER; note = thymine
  modified_base 49: mod_base = OTHER; note = thymine
  modified_base 52..53: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
attttggtactcttgatagtgaatctatcaaaataaggcaaaatgccgtggttatcccgtcaacttgttggcgagatttt

SEQ ID NO: 41 - RNA, 80 nt, synthetic construct
  modified_base 2..4: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 41: mod_base = OTHER; note = thymine
  modified_base 44: mod_base = OTHER; note = thymine
  modified_base 49: mod_base = OTHER; note = thymine
  modified_base 52: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
ctttcagtactctggagacagaatctactaaaagaaggcgtaatgccgtggtcaacacgtcaacttgttggcgagatttt

SEQ ID NO: 42 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 22: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 41: mod_base = OTHER; note = thymine
  modified_base 44: mod_base = OTHER; note = thymine
  modified_base 52..53: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
cttttagtactctgggaacagtctctactaaaagaaggcataatgccgaggttatgccgtcaacttgttggcgagatttt

SEQ ID NO: 43 - RNA, 80 nt, synthetic construct
  modified_base 2..5: mod_base = OTHER; note = thymine
  modified_base 8: mod_base = OTHER; note = thymine
  modified_base 12..14: mod_base = OTHER; note = thymine
  modified_base 24: mod_base = OTHER; note = thymine
  modified_base 29: mod_base = OTHER; note = thymine
  modified_base 32: mod_base = OTHER; note = thymine
  modified_base 35: mod_base = OTHER; note = thymine
  modified_base 51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
cttttagtgcgtttgcaaaggaatcaagtactagtggccaagcagcagagtacgtcacgtcaacttgttggcgagatttt

SEQ ID NO: 44 - RNA, 80 nt, synthetic construct
  modified_base 4: mod_base = OTHER; note = thymine
  modified_base 8: mod_base = OTHER; note = thymine
  modified_base 13: mod_base = OTHER; note = thymine
  modified_base 16: mod_base = OTHER; note = thymine
  modified_base 23: mod_base = OTHER; note = thymine
  modified_base 26: mod_base = OTHER; note = thymine
  modified_base 29: mod_base = OTHER; note = thymine
  modified_base 35: mod_base = OTHER; note = thymine
  modified_base 37: mod_base = OTHER; note = thymine
  modified_base 41..42: mod_base = OTHER; note = thymine
  modified_base 44: mod_base = OTHER; note = thymine
  modified_base 46: mod_base = OTHER; note = thymine
  modified_base 49: mod_base = OTHER; note = thymine
  modified_base 54..55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gcatgggtgacctggtgagagatagtcgtaaagctgtggattatgtcgtggggttcccgtcaacttgttggcgagatttt

SEQ ID NO: 45 - RNA, 80 nt, synthetic construct
  modified_base 4..5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 19: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 31..33: mod_base = OTHER; note = thymine
  modified_base 44: mod_base = OTHER; note = thymine
  modified_base 49: mod_base = OTHER; note = thymine
  modified_base 52..55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gcgttagtgctgtggacatagaatctcctgtttcagcgcaaaatggggtgcttttgccgtcaacttgttggcgagatttt

SEQ ID NO: 46 - RNA, 80 nt, synthetic construct
  modified_base 2: mod_base = OTHER; note = thymine
  modified_base 4: mod_base = OTHER; note = thymine
  modified_base 9: mod_base = OTHER; note = thymine
  modified_base 18: mod_base = OTHER; note = thymine
  modified_base 23: mod_base = OTHER; note = thymine
  modified_base 25: mod_base = OTHER; note = thymine
  modified_base 32: mod_base = OTHER; note = thymine
  modified_base 38: mod_base = OTHER; note = thymine
  modified_base 42..44: mod_base = OTHER; note = thymine
  modified_base 52: mod_base = OTHER; note = thymine
  modified_base 54..56: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtctcaactcgcagggataggatgtaagcgatccacgtaaatttgccgacatctttgcgtcaacttgttggcgagatttt

SEQ ID NO: 47 - RNA, 80 nt, synthetic construct
  modified_base 2: mod_base = OTHER; note = thymine
  modified_base 6: mod_base = OTHER; note = thymine
  modified_base 11: mod_base = OTHER; note = thymine
  modified_base 14: mod_base = OTHER; note = thymine
  modified_base 19..20: mod_base = OTHER; note = thymine
  modified_base 22..23: mod_base = OTHER; note = thymine
  modified_base 28: mod_base = OTHER; note = thymine
  modified_base 48..49: mod_base = OTHER; note = thymine
  modified_base 51..53: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtgcgtggaatactgggattgttccgatgaaaaaaagggacaacgcattgtttctcacgtcaacttgttggcgagatttt

SEQ ID NO: 48 - RNA, 80 nt, synthetic construct
  modified_base 2..3: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 26: mod_base = OTHER; note = thymine
  modified_base 29: mod_base = OTHER; note = thymine
  modified_base 31: mod_base = OTHER; note = thymine
  modified_base 42: mod_base = OTHER; note = thymine
  modified_base 44: mod_base = OTHER; note = thymine
  modified_base 51..53: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttagagtactctgggaacagaacctactataacaaggcgatatgccgcgtttataccgtcaacttgttggcgagatttt

SEQ ID NO: 49 - RNA, 80 nt, synthetic construct
  modified_base 2..3: mod_base = OTHER; note = thymine
  modified_base 6: mod_base = OTHER; note = thymine
  modified_base 11: mod_base = OTHER; note = thymine
  modified_base 30: mod_base = OTHER; note = thymine
  modified_base 35: mod_base = OTHER; note = thymine
  modified_base 39: mod_base = OTHER; note = thymine
  modified_base 42: mod_base = OTHER; note = thymine
  modified_base 47: mod_base = OTHER; note = thymine
  modified_base 49: mod_base = OTHER; note = thymine
  modified_base 51: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttagtggcctggggaaaaaggcgcagcctagactgggtgatccgatgtgtggggcgcgtcaacttgttggcgagatttt

SEQ ID NO: 50 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 26..29: mod_base = OTHER; note = thymine
  modified_base 31: mod_base = OTHER; note = thymine
  modified_base 41: mod_base = OTHER; note = thymine
  modified_base 44: mod_base = OTHER; note = thymine
  modified_base 49: mod_base = OTHER; note = thymine
  modified_base 52: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttatactactctggaaacggaaccttttataacaaggcataatgccgtggtcaaaccgtcaacttgttggcgagatttt

SEQ ID NO: 51 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 17: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 31: mod_base = OTHER; note = thymine
  modified_base 40: mod_base = OTHER; note = thymine
  modified_base 44: mod_base = OTHER; note = thymine
  modified_base 49: mod_base = OTHER; note = thymine
  modified_base 52..53: mod_base = OTHER; note = thymine
  modified_base 56: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttatagtactccgaatacggaatctagtataacaaggctaaatgccgtggttgctgcgtcaacttgttggcgagatttt

SEQ ID NO: 52 - RNA, 80 nt, synthetic construct
  modified_base 2..3: mod_base = OTHER; note = thymine
  modified_base 5: mod_base = OTHER; note = thymine
  modified_base 11: mod_base = OTHER; note = thymine
  modified_base 13: mod_base = OTHER; note = thymine
  modified_base 18: mod_base = OTHER; note = thymine
  modified_base 24: mod_base = OTHER; note = thymine
  modified_base 26..27: mod_base = OTHER; note = thymine
  modified_base 31: mod_base = OTHER; note = thymine
  modified_base 38: mod_base = OTHER; note = thymine
  modified_base 44: mod_base = OTHER; note = thymine
  modified_base 47: mod_base = OTHER; note = thymine
  modified_base 51: mod_base = OTHER; note = thymine
  modified_base 53: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttatgggactctgggatcagactcttccataacaaatcggaatgatgcatctatcccgtcaacttgttggcgagatttt

SEQ ID NO: 53 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 15: mod_base = OTHER; note = thymine
  modified_base 23: mod_base = OTHER; note = thymine
  modified_base 26: mod_base = OTHER; note = thymine
  modified_base 29: mod_base = OTHER; note = thymine
  modified_base 37: mod_base = OTHER; note = thymine
  modified_base 44: mod_base = OTHER; note = thymine
  modified_base 46: mod_base = OTHER; note = thymine
  modified_base 49: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttgtagtactctgtaaaaaggtcctggtaccagaatgcagagtgtcgtcttgctcacgtcaacttgttggcgagatttt

SEQ ID NO: 54 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11..12: mod_base = OTHER; note = thymine
  modified_base 20: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 49: mod_base = OTHER; note = thymine
  modified_base 53: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttgtagtacttgggaaactgaatctgctacaacaacgcaaaatgcggtgagtaccccgtcaacttgttggcgagatttt

SEQ ID NO: 55 - RNA, 80 nt, synthetic construct
  modified_base 2..3: mod_base = OTHER; note = thymine
  modified_base 5..6: mod_base = OTHER; note = thymine
  modified_base 8: mod_base = OTHER; note = thymine
  modified_base 10..11: mod_base = OTHER; note = thymine
  modified_base 13: mod_base = OTHER; note = thymine
  modified_base 26: mod_base = OTHER; note = thymine
  modified_base 40: mod_base = OTHER; note = thymine
  modified_base 42: mod_base = OTHER; note = thymine
  modified_base 44: mod_base = OTHER; note = thymine
  modified_base 49: mod_base = OTHER; note = thymine
  modified_base 52..53: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttgttgtattctggcgacagaggctacaacaacaaggctgtgtgccgtggttaaaccgtcaacttgttggcgagatttt

SEQ ID NO: 56 - RNA, 80 nt, synthetic construct
  modified_base 2..3: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 16: mod_base = OTHER; note = thymine
  modified_base 24: mod_base = OTHER; note = thymine
  modified_base 26: mod_base = OTHER; note = thymine
  modified_base 30..31: mod_base = OTHER; note = thymine
  modified_base 44: mod_base = OTHER; note = thymine
  modified_base 47: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttaacgtactctgctgacagaatctacgttaacaaagcgaagtgctgcgttcatcacgtcaacttgttggcgagatttt

SEQ ID NO: 57 - RNA, 80 nt, synthetic construct
  modified_base 2..3: mod_base = OTHER; note = thymine
  modified_base 5: mod_base = OTHER; note = thymine
  modified_base 11: mod_base = OTHER; note = thymine
  modified_base 13: mod_base = OTHER; note = thymine
  modified_base 15: mod_base = OTHER; note = thymine
  modified_base 19..20: mod_base = OTHER; note = thymine
  modified_base 24: mod_base = OTHER; note = thymine
  modified_base 26..27: mod_base = OTHER; note = thymine
  modified_base 44: mod_base = OTHER; note = thymine
  modified_base 49: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttgtccgactctataaattgaatcttggacaacaacgcaaaatgcggtgctcatcccgtcaacttgttggcgagatttt

SEQ ID NO: 58 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 15: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 40: mod_base = OTHER; note = thymine
  modified_base 42: mod_base = OTHER; note = thymine
  modified_base 44: mod_base = OTHER; note = thymine
  modified_base 49: mod_base = OTHER; note = thymine
  modified_base 52..53: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagtactctgtcgccagaatctactaaaacaacgctatatgcggtggttatcacgtcaacttgttggcgagatttt

SEQ ID NO: 59 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26..27: mod_base = OTHER; note = thymine
  modified_base 29: mod_base = OTHER; note = thymine
  modified_base 37: mod_base = OTHER; note = thymine
  modified_base 42: mod_base = OTHER; note = thymine
  modified_base 44: mod_base = OTHER; note = thymine
  modified_base 49: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
cttttagtactctggaaacggaatcttctaaaagaatgcgatatgcagtgttcatgccgtcaacttgttggcgagatttt

SEQ ID NO: 60 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 26: mod_base = OTHER; note = thymine
  modified_base 28: mod_base = OTHER; note = thymine
  modified_base 39: mod_base = OTHER; note = thymine
  modified_base 44: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttctggtactctggcaccagaacctatcagaacaaggtagaataccgggctcatcccgtcaacttgttggcgagatttt

SEQ ID NO: 61 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 16: mod_base = OTHER; note = thymine
  modified_base 23..24: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 49: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
cttttagtactccgctaacggattctactaaaagaacgcagaatgcggtgagcataccgtcaacttgttggcgagatttt

SEQ ID NO: 62 - RNA, 80 nt, synthetic construct
  modified_base 2..5: mod_base = OTHER; note = thymine
  modified_base 11: mod_base = OTHER; note = thymine
  modified_base 13: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 47: mod_base = OTHER; note = thymine
  modified_base 49: mod_base = OTHER; note = thymine
  modified_base 51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagcactctggaaacggaacctgctaaaacaaagcagaatgctgtgtccataacgtcaacttgttggcgagatttt

SEQ ID NO: 63 - RNA, 80 nt, synthetic construct
  modified_base 2..3: mod_base = OTHER; note = thymine
  modified_base 5: mod_base = OTHER; note = thymine
  modified_base 11: mod_base = OTHER; note = thymine
  modified_base 13: mod_base = OTHER; note = thymine
  modified_base 26..27: mod_base = OTHER; note = thymine
  modified_base 29: mod_base = OTHER; note = thymine
  modified_base 44: mod_base = OTHER; note = thymine
  modified_base 49: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttgtaggactctgaacacagaaccttctacaacaagccaaaatggcgtgctcatagcgtcaacttgttggcgagatttt

SEQ ID NO: 64 - RNA, 81 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 25: mod_base = OTHER; note = thymine
  modified_base 27: mod_base = OTHER; note = thymine
  modified_base 30: mod_base = OTHER; note = thymine
  modified_base 43: mod_base = OTHER; note = thymine
  modified_base 45: mod_base = OTHER; note = thymine
  modified_base 50..53: mod_base = OTHER; note = thymine
  modified_base 56: mod_base = OTHER; note = thymine
  modified_base 61: mod_base = OTHER; note = thymine
  modified_base 66..67: mod_base = OTHER; note = thymine
  modified_base 69..70: mod_base = OTHER; note = thymine
  modified_base 78..81: mod_base = OTHER; note = thymine
  misc_feature 1..81: note = gRNA
  source 1..81: mol_type = other RNA
gttgtagtactcggaaaaacggaatctactacaacaacgcagtatgcggttttcgtcacgtcaacttgttggcgagatttt

SEQ ID NO: 65 - RNA, 80 nt, synthetic construct
  modified_base 2..4: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 47: mod_base = OTHER; note = thymine
  modified_base 49: mod_base = OTHER; note = thymine
  modified_base 51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtttcaggactctcaacagggaatctcctgaaacaaagcacaatgctgtgtccatgccgtcaacttgttggcgagatttt

SEQ ID NO: 66 - RNA, 80 nt, synthetic construct
  modified_base 2..5: mod_base = OTHER; note = thymine
  modified_base 9: mod_base = OTHER; note = thymine
  modified_base 11: mod_base = OTHER; note = thymine
  modified_base 15: mod_base = OTHER; note = thymine
  modified_base 24: mod_base = OTHER; note = thymine
  modified_base 27: mod_base = OTHER; note = thymine
  modified_base 29: mod_base = OTHER; note = thymine
  modified_base 41: mod_base = OTHER; note = thymine
  modified_base 44: mod_base = OTHER; note = thymine
  modified_base 47: mod_base = OTHER; note = thymine
  modified_base 49: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttaggtctcggtaaaaagaatcatctaaaacagagcataatgctgtggtcatcccgtcaacttgttggcgagatttt

SEQ ID NO: 67 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 13..14: mod_base = OTHER; note = thymine
  modified_base 19: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 47: mod_base = OTHER; note = thymine
  modified_base 49: mod_base = OTHER; note = thymine
  modified_base 51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttgtagtactcttaaagtagaatctactacaacaaagcaagatgctgtgtgcatcccgtcaacttgttggcgagatttt

SEQ ID NO: 68 - RNA, 80 nt, synthetic construct
  modified_base 2..4: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 49: mod_base = OTHER; note = thymine
  modified_base 52..53: mod_base = OTHER; note = thymine
  modified_base 56: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtttcagtactgtggaaacacaatctactgaaacaacgcaaaatgcggtgcttagtccgtcaacttgttggcgagatttt

SEQ ID NO: 69 - RNA, 80 nt, synthetic construct
  modified_base 2..5: mod_base = OTHER; note = thymine
  modified_base 8: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 37: mod_base = OTHER; note = thymine
  modified_base 44: mod_base = OTHER; note = thymine
  modified_base 49: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagtacgctggaaacagcatctactaaaacaatgcaaagtgcagtgctcatcccgtcaacttgttggcgagatttt

SEQ ID NO: 70 - RNA, 80 nt, synthetic construct
  modified_base 2..4: mod_base = OTHER; note = thymine
  modified_base 11: mod_base = OTHER; note = thymine
  modified_base 13: mod_base = OTHER; note = thymine
  modified_base 26: mod_base = OTHER; note = thymine
  modified_base 29: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtttcagcactctgggaaaagaacctgctgaaacaacgcaaaacgcggagttcgtcacgtcaacttgttggcgagatttt

SEQ ID NO: 71 - RNA, 80 nt, synthetic construct
  modified_base 2..5: mod_base = OTHER; note = thymine
  modified_base 11: mod_base = OTHER; note = thymine
  modified_base 13: mod_base = OTHER; note = thymine
  modified_base 15: mod_base = OTHER; note = thymine
  modified_base 24: mod_base = OTHER; note = thymine
  modified_base 26..27: mod_base = OTHER; note = thymine
  modified_base 29: mod_base = OTHER; note = thymine
  modified_base 44: mod_base = OTHER; note = thymine
  modified_base 49..50: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttaggactctctaaggagaatcttctaaaacaacgcggaatgcggttctcatcccgtcaacttgttggcgagatttt

SEQ ID NO: 72 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 34: mod_base = OTHER; note = thymine
  modified_base 40: mod_base = OTHER; note = thymine
  modified_base 44: mod_base = OTHER; note = thymine
  modified_base 51..52: mod_base = OTHER; note = thymine
  modified_base 56: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagtactctgggaacagaatctactaaaataacgctgaatgcggggttcgctacgtcaacttgttggcgagatttt

SEQ ID NO: 73 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 46: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagtactctgaaaacggaacctactaaaacaagacgagatgtcgagctcatgccgtcaacttgttggcgagatttt

SEQ ID NO: 74 - RNA, 80 nt, synthetic construct
  modified_base 2..4: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 16: mod_base = OTHER; note = thymine
  modified_base 24: mod_base = OTHER; note = thymine
  modified_base 26: mod_base = OTHER; note = thymine
  modified_base 28..29: mod_base = OTHER; note = thymine
  modified_base 42: mod_base = OTHER; note = thymine
  modified_base 44: mod_base = OTHER; note = thymine
  modified_base 51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtttcagtactctggtaacagaatctattgaaacaaggcaatatgccgggtgcatcccgtcaacttgttggcgagatttt

SEQ ID NO: 75 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11..13: mod_base = OTHER; note = thymine
  modified_base 18: mod_base = OTHER; note = thymine
  modified_base 26: mod_base = OTHER; note = thymine
  modified_base 28..29: mod_base = OTHER; note = thymine
  modified_base 44: mod_base = OTHER; note = thymine
  modified_base 47: mod_base = OTHER; note = thymine
  modified_base 49: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttgtagtactttgaaatcagaacctattacaacaaagcagaatgctgtgttcgtcacgtcaacttgttggcgagatttt

SEQ ID NO: 76 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 41: mod_base = OTHER; note = thymine
  modified_base 44: mod_base = OTHER; note = thymine
  modified_base 46: mod_base = OTHER; note = thymine
  modified_base 51..52: mod_base = OTHER; note = thymine
  modified_base 55..56: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttctagtactctcgaaacagaatctactagaacaagacatgatgtcgcgttcattccgtcaacttgttggcgagatttt

SEQ ID NO: 77 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13..14: mod_base = OTHER; note = thymine
  modified_base 23..24: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagtactcttgaaggagattctactaaaacaacgcaaaatgcggcgtgcatcacgtcaacttgttggcgagatttt

SEQ ID NO: 78 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13..14: mod_base = OTHER; note = thymine
  modified_base 18: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 37: mod_base = OTHER; note = thymine
  modified_base 41: mod_base = OTHER; note = thymine
  modified_base 44: mod_base = OTHER; note = thymine
  modified_base 49: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagtactcttgaatcagaatctactaaaacagtgcctaatgcagtggtcatcccgtcaacttgttggcgagatttt

SEQ ID NO: 79 - RNA, 80 nt, synthetic construct
  modified_base 2..5: mod_base = OTHER; note = thymine
  modified_base 11: mod_base = OTHER; note = thymine
  modified_base 20: mod_base = OTHER; note = thymine
  modified_base 24: mod_base = OTHER; note = thymine
  modified_base 29: mod_base = OTHER; note = thymine
  modified_base 37: mod_base = OTHER; note = thymine
  modified_base 44: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagcgctcaggaaactgaatccactaaaacaatgcaaaatgcagcgttcatcccgtcaacttgttggcgagatttt

SEQ ID NO: 80 - RNA, 80 nt, synthetic construct
  modified_base 2..3: mod_base = OTHER; note = thymine
  modified_base 5..6: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 19: mod_base = OTHER; note = thymine
  modified_base 26: mod_base = OTHER; note = thymine
  modified_base 40..42: mod_base = OTHER; note = thymine
  modified_base 44: mod_base = OTHER; note = thymine
  modified_base 49: mod_base = OTHER; note = thymine
  modified_base 51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttgttgtactctagaagtagaacctacaacaacaaggctttatgccgtgtgcatcacgtcaacttgttggcgagatttt

SEQ ID NO: 81 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 19: mod_base = OTHER; note = thymine
  modified_base 24: mod_base = OTHER; note = thymine
  modified_base 26: mod_base = OTHER; note = thymine
  modified_base 28..29: mod_base = OTHER; note = thymine
  modified_base 44: mod_base = OTHER; note = thymine
  modified_base 46: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttaatactgcagaaatgcaatctattaaaacaagacaaaatgtcgggctcatcacgtcaacttgttggcgagatttt

SEQ ID NO: 82 - RNA, 80 nt, synthetic construct
  modified_base 2..4: mod_base = OTHER; note = thymine
  modified_base 7..8: mod_base = OTHER; note = thymine
  modified_base 11: mod_base = OTHER; note = thymine
  modified_base 13: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 49: mod_base = OTHER; note = thymine
  modified_base 51..53: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtttcattactctggaaacaggacctaatgaaacaacgcaaaatgcggtgtttatgccgtcaacttgttggcgagatttt

SEQ ID NO: 83 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 14: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 46: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagtactcctgacacgggatctgctaaaacaagacaaaatgtcgcgttcatcacgtcaacttgttggcgagatttt

SEQ ID NO: 84 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 20: mod_base = OTHER; note = thymine
  modified_base 26: mod_base = OTHER; note = thymine
  modified_base 29: mod_base = OTHER; note = thymine
  modified_base 40: mod_base = OTHER; note = thymine
  modified_base 42: mod_base = OTHER; note = thymine
  modified_base 44: mod_base = OTHER; note = thymine
  modified_base 49: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttggtactctgcgaaatgaacctactaaaacaaggctgtgtgccgtgttcatcacgtcaacttgttggcgagatttt

SEQ ID NO: 85 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 18: mod_base = OTHER; note = thymine
  modified_base 24: mod_base = OTHER; note = thymine
  modified_base 26: mod_base = OTHER; note = thymine
  modified_base 40: mod_base = OTHER; note = thymine
  modified_base 44: mod_base = OTHER; note = thymine
  modified_base 49: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttggtactctggaatcggaatctaccaaaacaaggctgaatgccgtcttcatcacgtcaacttgttggcgagatttt

SEQ ID NO: 86 - RNA, 80 nt, synthetic construct
  modified_base 2..3: mod_base = OTHER; note = thymine
  modified_base 5: mod_base = OTHER; note = thymine
  modified_base 11: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 41: mod_base = OTHER; note = thymine
  modified_base 44: mod_base = OTHER; note = thymine
  modified_base 49: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttctagcactccgggaacggaatctgctaaaacaacgcataatgcggtgttcatcccgtcaacttgttggcgagatttt

SEQ ID NO: 87 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 10: mod_base = OTHER; note = thymine
  modified_base 13: mod_base = OTHER; note = thymine
  modified_base 24..26: mod_base = OTHER; note = thymine
  modified_base 29: mod_base = OTHER; note = thymine
  modified_base 31: mod_base = OTHER; note = thymine
  modified_base 40: mod_base = OTHER; note = thymine
  modified_base 44: mod_base = OTHER; note = thymine
  modified_base 51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttatgctatgctggacacagcgtttagtataacaacgctaaatgcgggatgcatcccgtcaacttgttggcgagatttt

SEQ ID NO: 88 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 15: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 41: mod_base = OTHER; note = thymine
  modified_base 44: mod_base = OTHER; note = thymine
  modified_base 49..52: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttctagtactcagtaaaaagaatctactagaacaacgcataatgcggttttcaccccgtcaacttgttggcgagatttt

SEQ ID NO: 89 - RNA, 80 nt, synthetic construct
  modified_base 2..4: mod_base = OTHER; note = thymine
  modified_base 8: mod_base = OTHER; note = thymine
  modified_base 12..13: mod_base = OTHER; note = thymine
  modified_base 24: mod_base = OTHER; note = thymine
  modified_base 26: mod_base = OTHER; note = thymine
  modified_base 30: mod_base = OTHER; note = thymine
  modified_base 40: mod_base = OTHER; note = thymine
  modified_base 42: mod_base = OTHER; note = thymine
  modified_base 44: mod_base = OTHER; note = thymine
  modified_base 47: mod_base = OTHER; note = thymine
  modified_base 49: mod_base = OTHER; note = thymine
  modified_base 51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtttaggtaccttcgaaagaggatctacctaaacaaagctatgtgctgtgtgcatcacgtcaacttgttggcgagatttt

SEQ ID NO: 90 - RNA, 81 nt, synthetic construct
  modified_base 2..5: mod_base = OTHER; note = thymine
  modified_base 8: mod_base = OTHER; note = thymine
  modified_base 13: mod_base = OTHER; note = thymine
  modified_base 25: mod_base = OTHER; note = thymine
  modified_base 27: mod_base = OTHER; note = thymine
  modified_base 30: mod_base = OTHER; note = thymine
  modified_base 41: mod_base = OTHER; note = thymine
  modified_base 45: mod_base = OTHER; note = thymine
  modified_base 48: mod_base = OTHER; note = thymine
  modified_base 52..53: mod_base = OTHER; note = thymine
  modified_base 61: mod_base = OTHER; note = thymine
  modified_base 66..67: mod_base = OTHER; note = thymine
  modified_base 69..70: mod_base = OTHER; note = thymine
  modified_base 78..81: mod_base = OTHER; note = thymine
  misc_feature 1..81: note = gRNA
  source 1..81: mol_type = other RNA
gttttagtaccctgaaaccgaggatctactaaaacagagctacgtgctgagttcaacccgtcaacttgttggcgagatttt

SEQ ID NO: 91 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagtactctggcaacagaatctactaaaacaacgcaaaatgcggggctcatcccgtcaacttgttggcgagatttt

SEQ ID NO: 92 - RNA, 80 nt, synthetic construct
  modified_base 2..5: mod_base = OTHER; note = thymine
  modified_base 8: mod_base = OTHER; note = thymine
  modified_base 10: mod_base = OTHER; note = thymine
  modified_base 13: mod_base = OTHER; note = thymine
  modified_base 25..26: mod_base = OTHER; note = thymine
  modified_base 29: mod_base = OTHER; note = thymine
  modified_base 40..41: mod_base = OTHER; note = thymine
  modified_base 44: mod_base = OTHER; note = thymine
  modified_base 47: mod_base = OTHER; note = thymine
  modified_base 49: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagtatcctggaaacagaacttactaaaacaaagcttcatgctgtgctcatgccgtcaacttgttggcgagatttt

SEQ ID NO: 93 - RNA, 81 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 15: mod_base = OTHER; note = thymine
  modified_base 20: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 45: mod_base = OTHER; note = thymine
  modified_base 47: mod_base = OTHER; note = thymine
  modified_base 52..53: mod_base = OTHER; note = thymine
  modified_base 56: mod_base = OTHER; note = thymine
  modified_base 61: mod_base = OTHER; note = thymine
  modified_base 66..67: mod_base = OTHER; note = thymine
  modified_base 69..70: mod_base = OTHER; note = thymine
  modified_base 78..81: mod_base = OTHER; note = thymine
  misc_feature 1..81: note = gRNA
  source 1..81: mol_type = other RNA
gttttagtactcagtaaaatgaatctactaaaacaagacaaagatgtcgcgttcatcacgtcaacttgttggcgagatttt

SEQ ID NO: 94 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 41: mod_base = OTHER; note = thymine
  modified_base 44: mod_base = OTHER; note = thymine
  modified_base 50..51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttgtagtactctggcaacagaatctactgcaacaacgcatgatgcgggttccatgccgtcaacttgttggcgagatttt

SEQ ID NO: 95 - RNA, 80 nt, synthetic construct
  modified_base 2..4: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 15..16: mod_base = OTHER; note = thymine
  modified_base 24: mod_base = OTHER; note = thymine
  modified_base 26: mod_base = OTHER; note = thymine
  modified_base 37: mod_base = OTHER; note = thymine
  modified_base 41: mod_base = OTHER; note = thymine
  modified_base 44: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtttcggtactctgttaacaggatctaccgaaacaatgcgtaatgcagcgttcatcacgtcaacttgttggcgagatttt

SEQ ID NO: 96 - RNA, 80 nt, synthetic construct
  modified_base 2..4: mod_base = OTHER; note = thymine
  modified_base 8: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 47: mod_base = OTHER; note = thymine
  modified_base 49: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
atttgagtaccctggaaacaggatctactcaaacaaagcaaaatgctgtggtcatcacgtcaacttgttggcgagatttt

SEQ ID NO: 97 - RNA, 80 nt, synthetic construct
  modified_base 2: mod_base = OTHER; note = thymine
  modified_base 4..6: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 40: mod_base = OTHER; note = thymine
  modified_base 44: mod_base = OTHER; note = thymine
  modified_base 47: mod_base = OTHER; note = thymine
  modified_base 49: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtctttgtactctgggaacagaatctacgaagacaaagctgaatgctgtcttcatcacgtcaacttgttggcgagatttt

SEQ ID NO: 98 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 28..29: mod_base = OTHER; note = thymine
  modified_base 40: mod_base = OTHER; note = thymine
  modified_base 43..44: mod_base = OTHER; note = thymine
  modified_base 49: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttgtaatactgtgaaggcacaatctattacaacaacgctgattgcggtggtcataccgtcaacttgttggcgagatttt

SEQ ID NO: 99 - RNA, 80 nt, synthetic construct
  modified_base 2..4: mod_base = OTHER; note = thymine
  modified_base 8: mod_base = OTHER; note = thymine
  modified_base 11: mod_base = OTHER; note = thymine
  modified_base 14: mod_base = OTHER; note = thymine
  modified_base 18..19: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtttgactactcctgaattggaatctactcaaacaacgcaaaatgcggggctcatcccgtcaacttgttggcgagatttt

SEQ ID NO: 100 - RNA, 80 nt, synthetic construct
  modified_base 3: mod_base = OTHER; note = thymine
  modified_base 5: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26..27: mod_base = OTHER; note = thymine
  modified_base 29: mod_base = OTHER; note = thymine
  modified_base 40: mod_base = OTHER; note = thymine
  modified_base 49: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gctgtagaactctgaagccagaatcttctgcagcaacgctaaaggcggtgttcatcacgtcaacttgttggcgagatttt

SEQ ID NO: 101 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttgtagtactcgggaaaccgaatctgctacaacagcgcacaacgcggcgttcatcccgtcaacttgttggcgagatttt

SEQ ID NO: 102 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 37: mod_base = OTHER; note = thymine
  modified_base 41: mod_base = OTHER; note = thymine
  modified_base 44: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttgtagtactgtggcgacacaatctactacaacaatgcataatgcaggattcgtcgcgtcaacttgttggcgagatttt

SEQ ID NO: 103 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 31: mod_base = OTHER; note = thymine
  modified_base 38: mod_base = OTHER; note = thymine
  modified_base 41..42: mod_base = OTHER; note = thymine
  modified_base 44: mod_base = OTHER; note = thymine
  modified_base 49: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttatagtactccgcagacggaatctactataacaagtcattatggcgtagtcatcccgtcaacttgttggcgagatttt

SEQ ID NO: 104 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 23: mod_base = OTHER; note = thymine
  modified_base 26: mod_base = OTHER; note = thymine
  modified_base 29: mod_base = OTHER; note = thymine
  modified_base 40: mod_base = OTHER; note = thymine
  modified_base 44: mod_base = OTHER; note = thymine
  modified_base 47: mod_base = OTHER; note = thymine
  modified_base 51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttctagtactctggaaacggatcctgctagaacaaagctaaatgctgcgtccatagcgtcaacttgttggcgagatttt

SEQ ID NO: 105 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 18: mod_base = OTHER; note = thymine
  modified_base 20: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagtactcgggaatctgaaactactaaaacaacgcaaaatgcggcgtccatcccgtcaacttgttggcgagatttt

SEQ ID NO: 106 - RNA, 80 nt, synthetic construct
  modified_base 2..5: mod_base = OTHER; note = thymine
  modified_base 8: mod_base = OTHER; note = thymine
  modified_base 12..13: mod_base = OTHER; note = thymine
  modified_base 18: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 49: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagtaacttggactcaggatctactaaaacaacgcagaatgcggtgttcctcccgtcaacttgttggcgagatttt

SEQ ID NO: 107 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 18: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 37: mod_base = OTHER; note = thymine
  modified_base 42: mod_base = OTHER; note = thymine
  modified_base 44: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttggtactccgcaatcggaatctactaaaacaatgcaatgtgcagcgttcatcccgtcaacttgttggcgagatttt

SEQ ID NO: 108 - RNA, 80 nt, synthetic construct
  modified_base 2..4: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 15..16: mod_base = OTHER; note = thymine
  modified_base 24: mod_base = OTHER; note = thymine
  modified_base 29: mod_base = OTHER; note = thymine
  modified_base 41: mod_base = OTHER; note = thymine
  modified_base 44: mod_base = OTHER; note = thymine
  modified_base 46: mod_base = OTHER; note = thymine
  modified_base 49: mod_base = OTHER; note = thymine
  modified_base 51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtttgagtgctctgttagcggaatccgctcaaacaagacatgatgtcgtgtccatcccgtcaacttgttggcgagatttt

SEQ ID NO: 109 - RNA, 80 nt, synthetic construct
  modified_base 2: mod_base = OTHER; note = thymine
  modified_base 4..5: mod_base = OTHER; note = thymine
  modified_base 8..9: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 42: mod_base = OTHER; note = thymine
  modified_base 44: mod_base = OTHER; note = thymine
  modified_base 50: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtcttagttctctggaaacagaatctactaagacaacgcaatatgcgggtatcatcacgtcaacttgttggcgagatttt

SEQ ID NO: 110 - RNA, 80 nt, synthetic construct
  modified_base 2..5: mod_base = OTHER; note = thymine
  modified_base 13: mod_base = OTHER; note = thymine
  modified_base 22: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 47: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagcacactggaaacggtacctgctaaaacaaagcaaaatgctggggtcatcacgtcaacttgttggcgagatttt

SEQ ID NO: 111 - RNA, 80 nt, synthetic construct
  modified_base 1..4: mod_base = OTHER; note = thymine
  modified_base 11: mod_base = OTHER; note = thymine
  modified_base 13: mod_base = OTHER; note = thymine
  modified_base 15: mod_base = OTHER; note = thymine
  modified_base 17..18: mod_base = OTHER; note = thymine
  modified_base 26..27: mod_base = OTHER; note = thymine
  modified_base 29: mod_base = OTHER; note = thymine
  modified_base 37: mod_base = OTHER; note = thymine
  modified_base 44: mod_base = OTHER; note = thymine
  modified_base 49: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
ttttcagaactctgtcttcagaaccttctgaaaaagtgcagcatgcagtactcatcacgtcaacttgttggcgagatttt

SEQ ID NO: 112 - RNA, 80 nt, synthetic construct
  modified_base 2: mod_base = OTHER; note = thymine
  modified_base 8: mod_base = OTHER; note = thymine
  modified_base 11..13: mod_base = OTHER; note = thymine
  modified_base 15: mod_base = OTHER; note = thymine
  modified_base 24: mod_base = OTHER; note = thymine
  modified_base 26: mod_base = OTHER; note = thymine
  modified_base 29..31: mod_base = OTHER; note = thymine
  modified_base 40: mod_base = OTHER; note = thymine
  modified_base 44: mod_base = OTHER; note = thymine
  modified_base 49: mod_base = OTHER; note = thymine
  modified_base 51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
ctcaaagtactttgtaaacagaatctactttgagaacgctaagtgcggtgtccatcccgtcaacttgttggcgagatttt

SEQ ID NO: 113 - RNA, 80 nt, synthetic construct
  modified_base 2..3: mod_base = OTHER; note = thymine
  modified_base 8: mod_base = OTHER; note = thymine
  modified_base 11: mod_base = OTHER; note = thymine
  modified_base 16..17: mod_base = OTHER; note = thymine
  modified_base 20: mod_base = OTHER; note = thymine
  modified_base 24: mod_base = OTHER; note = thymine
  modified_base 26: mod_base = OTHER; note = thymine
  modified_base 28..29: mod_base = OTHER; note = thymine
  modified_base 44: mod_base = OTHER; note = thymine
  modified_base 46: mod_base = OTHER; note = thymine
  modified_base 49: mod_base = OTHER; note = thymine
  modified_base 51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttcgagtactcaggttactgaatctattcgaacaagacaaaatgtcgtgtccatcacgtcaacttgttggcgagatttt

SEQ ID NO: 114 - RNA, 80 nt, synthetic construct
  modified_base 2..3: mod_base = OTHER; note = thymine
  modified_base 5..6: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 26: mod_base = OTHER; note = thymine
  modified_base 42: mod_base = OTHER; note = thymine
  modified_base 44: mod_base = OTHER; note = thymine
  modified_base 46: mod_base = OTHER; note = thymine
  modified_base 49: mod_base = OTHER; note = thymine
  modified_base 51: mod_base = OTHER; note = thymine
  modified_base 55..56: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttgttgtactctagaaacagaacctacagcaacaagaccgtgtgtcgtgtccattacgtcaacttgttggcgagatttt

SEQ ID NO: 115 - RNA, 80 nt, synthetic construct
  modified_base 2..4: mod_base = OTHER; note = thymine
  modified_base 11: mod_base = OTHER; note = thymine
  modified_base 13: mod_base = OTHER; note = thymine
  modified_base 18: mod_base = OTHER; note = thymine
  modified_base 24: mod_base = OTHER; note = thymine
  modified_base 26..27: mod_base = OTHER; note = thymine
  modified_base 29..30: mod_base = OTHER; note = thymine
  modified_base 44: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtttaaggactgtggactcacaatcttcttaaacaacgcaaaatgcggggttcatcacgtcaacttgttggcgagatttt

SEQ ID NO: 116 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 16: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 41: mod_base = OTHER; note = thymine
  modified_base 44: mod_base = OTHER; note = thymine
  modified_base 47: mod_base = OTHER; note = thymine
  modified_base 49: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttactactctggtaacagaatctagtaaaacagagcataatgctgtgttcgtcccgtcaacttgttggcgagatttt

SEQ ID NO: 117 - RNA, 80 nt, synthetic construct
  modified_base 2..4: mod_base = OTHER; note = thymine
  modified_base 8: mod_base = OTHER; note = thymine
  modified_base 13: mod_base = OTHER; note = thymine
  modified_base 16: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 34: mod_base = OTHER; note = thymine
  modified_base 44: mod_base = OTHER; note = thymine
  modified_base 49: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
atttccgtaagctggtagcagcatctactgaaataacgcgaaatgcggtgttcatcccgtcaacttgttggcgagatttt

SEQ ID NO: 118 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 12..13: mod_base = OTHER; note = thymine
  modified_base 26: mod_base = OTHER; note = thymine
  modified_base 31: mod_base = OTHER; note = thymine
  modified_base 37: mod_base = OTHER; note = thymine
  modified_base 44: mod_base = OTHER; note = thymine
  modified_base 49: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttatggtaccttggaaacaagacctacgataacaatgcgaaatgcagtggtcatcacgtcaacttgttggcgagatttt

SEQ ID NO: 119 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 40: mod_base = OTHER; note = thymine
  modified_base 44: mod_base = OTHER; note = thymine
  modified_base 49: mod_base = OTHER; note = thymine
  modified_base 55..56: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttgtagtactctcgaaacggaatctactgcaacaacgctaaatgcggtgaccattacgtcaacttgttggcgagatttt

SEQ ID NO: 120 - RNA, 80 nt, synthetic construct
  modified_base 2..5: mod_base = OTHER; note = thymine
  modified_base 11: mod_base = OTHER; note = thymine
  modified_base 13: mod_base = OTHER; note = thymine
  modified_base 15: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttaccactctgtacccagaatctggtaaaacaacgcaaaatgcggcgttcatcacgtcaacttgttggcgagatttt

SEQ ID NO: 121 - RNA, 81 nt, synthetic construct
  modified_base 2..5: mod_base = OTHER; note = thymine
  modified_base 8: mod_base = OTHER; note = thymine
  modified_base 12: mod_base = OTHER; note = thymine
  modified_base 14: mod_base = OTHER; note = thymine
  modified_base 25: mod_base = OTHER; note = thymine
  modified_base 27: mod_base = OTHER; note = thymine
  modified_base 30: mod_base = OTHER; note = thymine
  modified_base 38: mod_base = OTHER; note = thymine
  modified_base 45: mod_base = OTHER; note = thymine
  modified_base 50: mod_base = OTHER; note = thymine
  modified_base 53..54: mod_base = OTHER; note = thymine
  modified_base 56: mod_base = OTHER; note = thymine
  modified_base 61: mod_base = OTHER; note = thymine
  modified_base 66..67: mod_base = OTHER; note = thymine
  modified_base 69..70: mod_base = OTHER; note = thymine
  modified_base 78..81: mod_base = OTHER; note = thymine
  misc_feature 1..81: note = gRNA
  source 1..81: mol_type = other RNA
gttttagtacgtctggacgcagactctactgaaacagtgcaaaatgcagtggttatcccgtcaacttgttggcgagatttt

SEQ ID NO: 122 - RNA, 80 nt, synthetic construct
  modified_base 1..3: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 26: mod_base = OTHER; note = thymine
  modified_base 29..30: mod_base = OTHER; note = thymine
  modified_base 44: mod_base = OTHER; note = thymine
  modified_base 46: mod_base = OTHER; note = thymine
  modified_base 51..52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
tttgaagtactctcgaaacagaagctacttcaaaaagacagaatgtcgcgttcatcacgtcaacttgttggcgagatttt

SEQ ID NO: 123 - RNA, 80 nt, synthetic construct
  modified_base 2..3: mod_base = OTHER; note = thymine
  modified_base 5: mod_base = OTHER; note = thymine
  modified_base 9: mod_base = OTHER; note = thymine
  modified_base 11: mod_base = OTHER; note = thymine
  modified_base 13..14: mod_base = OTHER; note = thymine
  modified_base 24: mod_base = OTHER; note = thymine
  modified_base 29: mod_base = OTHER; note = thymine
  modified_base 31: mod_base = OTHER; note = thymine
  modified_base 37: mod_base = OTHER; note = thymine
  modified_base 44: mod_base = OTHER; note = thymine
  modified_base 49: mod_base = OTHER; note = thymine
  modified_base 51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttatggctctcttgacaaggaatcagctataacaatgcaaaatgcagtgtgcatcacgtcaacttgttggcgagatttt

SEQ ID NO: 124 - RNA, 80 nt, synthetic construct
  modified_base 2..3: mod_base = OTHER; note = thymine
  modified_base 8: mod_base = OTHER; note = thymine
  modified_base 13: mod_base = OTHER; note = thymine
  modified_base 18: mod_base = OTHER; note = thymine
  modified_base 24: mod_base = OTHER; note = thymine
  modified_base 26..27: mod_base = OTHER; note = thymine
  modified_base 31: mod_base = OTHER; note = thymine
  modified_base 44: mod_base = OTHER; note = thymine
  modified_base 51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttgcggtaccctggaatcaggatcttccgtaacaacgcaaactgcggagtgcatcccgtcaacttgttggcgagatttt

SEQ ID NO: 125 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 13..14: mod_base = OTHER; note = thymine
  modified_base 17: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 49: mod_base = OTHER; note = thymine
  modified_base 52: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttgtagtactcttgatgcagaagctactgcaacaacgcaaaatgcggtgctcatagcgtcaacttgttggcgagatttt

SEQ ID NO: 126 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 37: mod_base = OTHER; note = thymine
  modified_base 44: mod_base = OTHER; note = thymine
  modified_base 51..52: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagtactctgaaagcagaatctactaaaacaatgcaacatgcagcgttcaccacgtcaacttgttggcgagatttt

SEQ ID NO: 127 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 44: mod_base = OTHER; note = thymine
  modified_base 49: mod_base = OTHER; note = thymine
  modified_base 51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagtactctgggaacagaatctactaaaacaacgcaacatgcggtatgcatagcgtcaacttgttggcgagatttt

SEQ ID NO: 128 - RNA, 80 nt, synthetic construct
  modified_base 2..5: mod_base = OTHER; note = thymine
  modified_base 8: mod_base = OTHER; note = thymine
  modified_base 12..13: mod_base = OTHER; note = thymine
  modified_base 17..18: mod_base = OTHER; note = thymine
  modified_base 24: mod_base = OTHER; note = thymine
  modified_base 26: mod_base = OTHER; note = thymine
  modified_base 34: mod_base = OTHER; note = thymine
  modified_base 44: mod_base = OTHER; note = thymine
  modified_base 49: mod_base = OTHER; note = thymine
  modified_base 52: mod_base = OTHER; note = thymine
  modified_base 56: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
attttcgtacgttcgattgaacatctacgaaaataacgcaaaatgcggtgatcactccgtcaacttgttggcgagatttt

SEQ ID NO: 129 - RNA, 80 nt, synthetic construct
  modified_base 2..3: mod_base = OTHER; note = thymine
  modified_base 5: mod_base = OTHER; note = thymine
  modified_base 8: mod_base = OTHER; note = thymine
  modified_base 11: mod_base = OTHER; note = thymine
  modified_base 18: mod_base = OTHER; note = thymine
  modified_base 20: mod_base = OTHER; note = thymine
  modified_base 24: mod_base = OTHER; note = thymine
  modified_base 26: mod_base = OTHER; note = thymine
  modified_base 28..29: mod_base = OTHER; note = thymine
  modified_base 44: mod_base = OTHER; note = thymine
  modified_base 47: mod_base = OTHER; note = thymine
  modified_base 52: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttctaatactcaaggatatgaatctattagaacagagcaaaatgctggggtcaaaccgtcaacttgttggcgagatttt

SEQ ID NO: 130 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 40: mod_base = OTHER; note = thymine
  modified_base 44: mod_base = OTHER; note = thymine
  modified_base 51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagtactctgaaaacagaatctactagaacaacgctgaatgcggggtacatcccgtcaacttgttggcgagatttt

SEQ ID NO: 131 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 20: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 40: mod_base = OTHER; note = thymine
  modified_base 44: mod_base = OTHER; note = thymine
  modified_base 47: mod_base = OTHER; note = thymine
  modified_base 49: mod_base = OTHER; note = thymine
  modified_base 52..53: mod_base = OTHER; note = thymine
  modified_base 56: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagtactctggaaactgaatctactaaaacaaagctgcgtgctgtagttaatgcgtcaacttgttggcgagatttt

SEQ ID NO: 132 - RNA, 80 nt, synthetic construct
  modified_base 2..5: mod_base = OTHER; note = thymine
  modified_base 8: mod_base = OTHER; note = thymine
  modified_base 13: mod_base = OTHER; note = thymine
  modified_base 16: mod_base = OTHER; note = thymine
  modified_base 22: mod_base = OTHER; note = thymine
  modified_base 24: mod_base = OTHER; note = thymine
  modified_base 26: mod_base = OTHER; note = thymine
  modified_base 40: mod_base = OTHER; note = thymine
  modified_base 44: mod_base = OTHER; note = thymine
  modified_base 46: mod_base = OTHER; note = thymine
  modified_base 49: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttggtacactagtgacagtatctaccaaaacaagactgcgtgtcgtgggcatcccgtcaacttgttggcgagatttt

SEQ ID NO: 133 - RNA, 80 nt, synthetic construct
  modified_base 2..4: mod_base = OTHER; note = thymine
  modified_base 8: mod_base = OTHER; note = thymine
  modified_base 12: mod_base = OTHER; note = thymine
  modified_base 16: mod_base = OTHER; note = thymine
  modified_base 18: mod_base = OTHER; note = thymine
  modified_base 26: mod_base = OTHER; note = thymine
  modified_base 28..29: mod_base = OTHER; note = thymine
  modified_base 44: mod_base = OTHER; note = thymine
  modified_base 49: mod_base = OTHER; note = thymine
  modified_base 51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gtttcagtacgtgcatatgcacacctattgaaacaaggcaaaatgccgtgtgcatcccgtcaacttgttggcgagatttt

SEQ ID NO: 134 - RNA, 80 nt, synthetic construct
  modified_base 2..5: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  modified_base 19: mod_base = OTHER; note = thymine
  modified_base 26: mod_base = OTHER; note = thymine
  modified_base 29: mod_base = OTHER; note = thymine
  modified_base 37: mod_base = OTHER; note = thymine
  modified_base 40: mod_base = OTHER; note = thymine
  modified_base 44: mod_base = OTHER; note = thymine
  modified_base 49: mod_base = OTHER; note = thymine
  modified_base 51: mod_base = OTHER; note = thymine
  modified_base 55: mod_base = OTHER; note = thymine
  modified_base 60: mod_base = OTHER; note = thymine
  modified_base 65..66: mod_base = OTHER; note = thymine
  modified_base 68..69: mod_base = OTHER; note = thymine
  modified_base 77..80: mod_base = OTHER; note = thymine
  misc_feature 1..80: note = gRNA
  source 1..80: mol_type = other RNA
gttttagtaatctcgaaatagaacctactaaaacaatgctaaatgcagtgtgcatcacgtcaacttgttggcgagatttt

SEQ ID NO: 135 - DNA, 42 bp, synthetic construct
  source 1..42: mol_type = other DNA
ggggataatacgactcactataggcgagggcgatgccaccta

SEQ ID NO: 136 - DNA, 23 bp, synthetic construct
  source 1..23: mol_type = other DNA
aaaatctcgccaacaagttgacg

SEQ ID NO: 137 - DNA, 94 bp, synthetic construct
  variation 25: note = n is 14%A,14%C,58%G,14%T
  variation 26: note = n is 58%A,14%C,14%G,14%T
  variation 27: note = n is 14%A,14%C,14%G,58%T
  variation 28: note = n is 58%A,14%C,14%G,14%T
  variation 29: note = n is 14%A,14%C,14%G,58%T
  variation 30: note = n is 58%A,14%C,14%G,14%T
  variation 31: note = n is 58%A,14%C,14%G,14%T
  variation 32: note = n is 14%A,58%C,14%G,14%T
  variation 33: note = n is 58%A,14%C,14%G,14%T
  variation 34: note = n is 14%A,14%C,58%G,14%T
  variation 35: note = n is 14%A,14%C,58%G,14%T
  variation 36: note = n is 14%A,58%C,14%G,14%T
  variation 37: note = n is 14%A,14%C,58%G,14%T
  variation 38: note = n is 14%A,14%C,58%G,14%T
  variation 39: note = n is 14%A,14%C,14%G,58%T
  variation 40: note = n is 14%A,58%C,14%G,14%T
  variation 41: note = n is 58%A,14%C,14%G,14%T
  variation 42: note = n is 14%A,14%C,14%G,58%T
  variation 43: note = n is 14%A,14%C,14%G,58%T
  variation 44: note = n is 14%A,14%C,14%G,58%T
  variation 45: note = n is 14%A,58%C,14%G,14%T
  variation 46: note = n is 14%A,58%C,14%G,14%T
  variation 47: note = n is 58%A,14%C,14%G,14%T
  variation 48: note = n is 14%A,14%C,58%G,14%T
  variation 49: note = n is 14%A,14%C,14%G,58%T
  variation 50: note = n is 14%A,14%C,14%G,58%T
  variation 51: note = n is 14%A,14%C,14%G,58%T
  variation 52: note = n is 58%A,14%C,14%G,14%T
  variation 53: note = n is 14%A,14%C,58%G,14%T
  variation 54: note = n is 14%A,14%C,58%G,14%T
  variation 55: note = n is 14%A,14%C,14%G,58%T
  variation 56: note = n is 58%A,14%C,14%G,14%T
  variation 57: note = n is 14%A,58%C,14%G,14%T
  variation 58: note = n is 14%A,14%C,58%G,14%T
  variation 59: note = n is 58%A,14%C,14%G,14%T
  variation 60: note = n is 14%A,58%C,14%G,14%T
  variation 61: note = n is 14%A,14%C,58%G,14%T
  variation 62: note = n is 14%A,14%C,58%G,14%T
  variation 63: note = n is 58%A,14%C,14%G,14%T
  variation 64: note = n is 14%A,14%C,14%G,58%T
  variation 65: note = n is 14%A,58%C,14%G,14%T
  variation 66: note = n is 14%A,14%C,58%G,14%T
  variation 67: note = n is 58%A,14%C,14%G,14%T
  variation 68: note = n is 14%A,14%C,14%G,58%T
  variation 69: note = n is 58%A,14%C,14%G,14%T
  variation 70: note = n is 14%A,58%C,14%G,14%T
  variation 71: note = n is 14%A,14%C,14%G,58%T
  variation 72: note = n is 58%A,14%C,14%G,14%T
  variation 73: note = n is 58%A,14%C,14%G,14%T
  variation 74: note = n is 58%A,14%C,14%G,14%T
  variation 75: note = n is 14%A,58%C,14%G,14%T
  source 1..94: mol_type = other DNA
aaaatctcgccaacaagttgacgcnnnnnnnnnnnnnnnnnnnnnnnnnnnnnnnnnnnnnnnnnnnnnnnnnnntaggtggcatcgccctcgc

SEQ ID NO: 138 - DNA, 20 bp, synthetic construct
  source 1..20: mol_type = other DNA
ggcgagggcgatgccaccta

SEQ ID NO: 139 - skipped

SEQ ID NO: 140 - DNA, 21 bp, synthetic construct
  source 1..21: mol_type = other DNA
gtgagcaagggcgaggagctg

SEQ ID NO: 141 - DNA, 22 bp, synthetic construct
  source 1..22: mol_type = other DNA
tacttgtacagctcgtccatgc

SEQ ID NO: 142 - DNA, 26 bp, synthetic construct
  source 1..26: mol_type = other DNA
ggcgagggcgatgccacctacggaat

SEQ ID NO: 143 - DNA, 21 bp, synthetic construct
  source 1..21: mol_type = other DNA
gtgagcaagggcgaggagctg

SEQ ID NO: 144 - DNA, 22 bp, synthetic construct
  source 1..22: mol_type = other DNA
tacttgtacagctcgtccatgc

SEQ ID NO: 145 - DNA, 23 bp, synthetic construct
  source 1..23: mol_type = other DNA
aaaatctcgccaacaagttgacg

SEQ ID NO: 146 - DNA, 42 bp, synthetic construct; identical to SEQ ID NO: 135
  source 1..42: mol_type = other DNA

SEQ ID NO: 147 - DNA, 23 bp, synthetic construct
  source 1..23: mol_type = other DNA
aaaatctcgccaacaagttgacg

SEQ ID NO: 148 - DNA, 38 bp, synthetic construct
  source 1..38: mol_type = other DNA
gaccacggtctcacaccgccggtggtgcagatgaactt

SEQ ID NO: 149 - DNA, 42 bp, synthetic construct
  source 1..42: mol_type = other DNA
gggttcctgcggccgcaaaaaaatctcgccaacaagttgacg

SEQ ID NO: 150 - DNA, 58 bp, synthetic construct
  source 1..58: mol_type = other DNA
acactctttccctacacgacgctcttccgatctggggataatacgactcactataggc

SEQ ID NO: 151 - DNA, 55 bp, synthetic construct
  source 1..55: mol_type = other DNA
gactggagttcagacgtgtgctcttccgatctaaaatctcgccaacaagttgacg

SEQ ID NO: 152 - DNA, 21 bp, synthetic construct
  source 1..21: mol_type = other DNA
aagggcgaggagctgttcacc

SEQ ID NO: 153 - DNA, 23 bp, synthetic construct
  source 1..23: mol_type = other DNA
cgtcctccttgaagtcgatgccc

SEQ ID NO: 154 - DNA, 21 bp, synthetic construct
  source 1..21: mol_type = other DNA
aagggcgaggagctgttcacc

SEQ ID NO: 155 - DNA, 23 bp, synthetic construct
  source 1..23: mol_type = other DNA
cgtcctccttgaagtcgatgccc

SEQ ID NO: 156 - DNA, 22 bp, synthetic construct
  source 1..22: mol_type = other DNA
gagaaccccgagtacttgacac

SEQ ID NO: 157 - DNA, 21 bp, synthetic construct
  source 1..21: mol_type = other DNA
ctcttgatgccagcagaagtc

SEQ ID NO: 158 - DNA, 22 bp, synthetic construct
  source 1..22: mol_type = other DNA
aaaagtcttgaacgaaagctgg

SEQ ID NO: 159 - DNA, 22 bp, synthetic construct
  source 1..22: mol_type = other DNA
caaagactgagaggacaagcct

SEQ ID NO: 160 - DNA, 22 bp, synthetic construct
  source 1..22: mol_type = other DNA
catggggttacctttttcagat

SEQ ID NO: 161 - DNA, 22 bp, synthetic construct
  source 1..22: mol_type = other DNA
ttgaacaggtctgactgagcat

SEQ ID NO: 162 - DNA, 21 bp, synthetic construct
  source 1..21: mol_type = other DNA
gtgagggcctggccggccctt

SEQ ID NO: 163 - DNA, 21 bp, synthetic construct
  source 1..21: mol_type = other DNA
ggcctggccaggaccacgctc

SEQ ID NO: 164 - DNA, 21 bp, synthetic construct
  source 1..21: mol_type = other DNA
gactgccagcggctggccagc

SEQ ID NO: 165 - DNA, 21 bp, synthetic construct
  source 1..21: mol_type = other DNA
ggcccgagctgcgccagcaga

SEQ ID NO: 166 - DNA, 21 bp, synthetic construct
  source 1..21: mol_type = other DNA
ggcctccccaaagcctggcca

SEQ ID NO: 167 - DNA, 21 bp, synthetic construct
  source 1..21: mol_type = other DNA
gacgtcgccgtccagctcgac

SEQ ID NO: 168 - DNA, 21 bp, synthetic construct
  source 1..21: mol_type = other DNA
gggccagagcaccgaggagct

SEQ ID NO: 169 - DNA, 21 bp, synthetic construct
  source 1..21: mol_type = other DNA
gatcctagcgtcgagccccct

SEQ ID NO: 170 - DNA, 21 bp, synthetic construct
  source 1..21: mol_type = other DNA
ggagaccggcgcacagaggaa

SEQ ID NO: 171 - DNA, 21 bp, synthetic construct
  source 1..21: mol_type = other DNA
gtccttgggggcttcccaagg

SEQ ID NO: 172 - DNA, 21 bp, synthetic construct
  source 1..21: mol_type = other DNA
gccacgggcggatgtcgttgt

SEQ ID NO: 173 - DNA, 21 bp, synthetic construct
  source 1..21: mol_type = other DNA
gacaccggagccttctgcgac

SEQ ID NO: 174 - DNA, 21 bp, synthetic construct
  source 1..21: mol_type = other DNA
gtctctacacccagggcaccg

SEQ ID NO: 175 - DNA, 21 bp, synthetic construct
  source 1..21: mol_type = other DNA
gtacggtcccctggccagtga

SEQ ID NO: 176 - DNA, 21 bp, synthetic construct
  source 1..21: mol_type = other DNA
gaagacctgccctgtgcagct

SEQ ID NO: 177 - DNA, 21 bp, synthetic construct
  source 1..21: mol_type = other DNA
gtttcccagcctggagcatcg

SEQ ID NO: 178 - DNA, 21 bp, synthetic construct
  source 1..21: mol_type = other DNA
gcggcagagaacccagagtac

SEQ ID NO: 179 - DNA, 21 bp, synthetic construct
  source 1..21: mol_type = other DNA
gcaacatcctggggcacaagc

SEQ ID NO: 180 - DNA, 21 bp, synthetic construct
  source 1..21: mol_type = other DNA
gccggtggtgcagatgaactt

SEQ ID NO: 181 - DNA, 21 bp, synthetic construct
  source 1..21: mol_type = other DNA
gtcattctgttctcagttttc

SEQ ID NO: 182 - DNA, 21 bp, synthetic construct
  source 1..21: mol_type = other DNA
gccggtggtgcagatgaactt

SEQ ID NO: 183 - DNA, 26 bp, synthetic construct
  source 1..26: mol_type = other DNA
tgagggcctggccggcccttgtgggt

SEQ ID NO: 184 - DNA, 26 bp, synthetic construct
  source 1..26: mol_type = other DNA
gcctggccaggaccacgctctcgggt

SEQ ID NO: 185 - DNA, 26 bp, synthetic construct
  source 1..26: mol_type = other DNA
actgccagcggctggccagccagggt

SEQ ID NO: 186 - DNA, 26 bp, synthetic construct
  source 1..26: mol_type = other DNA
gcccgagctgcgccagcagaccgagt

SEQ ID NO: 187 - DNA, 26 bp, synthetic construct
  source 1..26: mol_type = other DNA
gcctccccaaagcctggccagggagt

SEQ ID NO: 188 - DNA, 26 bp, synthetic construct
  source 1..26: mol_type = other DNA
acgtcgccgtccagctcgaccaggat

SEQ ID NO: 189 - DNA, 26 bp, synthetic construct
  source 1..26: mol_type = other DNA
ggccagagcaccgaggagctgcgggt

SEQ ID NO: 190 - DNA, 26 bp, synthetic construct
  source 1..26: mol_type = other DNA
atcctagcgtcgagccccctctgagt

SEQ ID NO: 191 - DNA, 26 bp, synthetic construct
  source 1..26: mol_type = other DNA
gagaccggcgcacagaggaagagaat

SEQ ID NO: 192 - DNA, 26 bp, synthetic construct
  source 1..26: mol_type = other DNA
tccttgggggcttcccaaggtggaat

SEQ ID NO: 193 - DNA, 26 bp, synthetic construct
  source 1..26: mol_type = other DNA
ccacgggcggatgtcgttgtccgggt

SEQ ID NO: 194 - DNA, 26 bp, synthetic construct
  source 1..26: mol_type = other DNA
acaccggagccttctgcgacgtgggt

SEQ ID NO: 195 - DNA, 26 bp, synthetic construct
  source 1..26: mol_type = other DNA
tctctacacccagggcaccggtgaat

SEQ ID NO: 196 - DNA, 26 bp, synthetic construct
  source 1..26: mol_type = other DNA
tacggtcccctggccagtgaaagggt

SEQ ID NO: 197 - DNA, 26 bp, synthetic construct
  source 1..26: mol_type = other DNA
aagacctgccctgtgcagctgtgggt

SEQ ID NO: 198 - DNA, 26 bp, synthetic construct
  source 1..26: mol_type = other DNA
tttcccagcctggagcatcggtgagt

SEQ ID NO: 199 - DNA, 26 bp, synthetic construct
  source 1..26: mol_type = other DNA
cggcagagaacccagagtacctgggt

SEQ ID NO: 200 - DNA, 26 bp, synthetic construct
  source 1..26: mol_type = other DNA
caacatcctggggcacaagctggagt

SEQ ID NO: 201 - DNA, 26 bp, synthetic construct
  source 1..26: mol_type = other DNA
ccggtggtgcagatgaacttcagggt

SEQ ID NO: 202 - DNA, 26 bp, synthetic construct
  source 1..26: mol_type = other DNA
tcattctgttctcagttttcctggat

SEQ ID NO: 203 - DNA, 26 bp, synthetic construct
  source 1..26: mol_type = other DNA
ccggtggtgcagatgaacttcagggt

SEQ ID NO: 204 - DNA, 21 bp, synthetic construct
  source 1..21: mol_type = other DNA
gctgccgtcctcgatgttgtg

SEQ ID NO: 205 - DNA, 21 bp, synthetic construct
  source 1..21: mol_type = other DNA
gcacctgggagccatttgcct

SEQ ID NO: 206 - DNA, 21 bp, synthetic construct
  source 1..21: mol_type = other DNA
gcaggtcacccaggaactgag

SEQ ID NO: 207 - DNA, 21 bp, synthetic construct
  source 1..21: mol_type = other DNA
ggcccctcctcagcatcttat

SEQ ID NO: 208 - DNA, 21 bp, synthetic construct
  source 1..21: mol_type = other DNA
gagttgtccctgagagtcagg

SEQ ID NO: 209 - DNA, 21 bp, synthetic construct
  source 1..21: mol_type = other DNA
gccggtggtgcagatgaactt

SEQ ID NO: 210 - DNA, 21 bp, synthetic construct
  source 1..21: mol_type = other DNA
ggtcgtgctgcttcatgtggt

SEQ ID NO: 211 - DNA, 21 bp, synthetic construct
  source 1..21: mol_type = other DNA
gagcatcccatggaacctgga

SEQ ID NO: 212 - DNA, 21 bp, synthetic construct
  source 1..21: mol_type = other DNA
gcttgccatgagccttcacct

SEQ ID NO: 213 - DNA, 21 bp, synthetic construct
  source 1..21: mol_type = other DNA
gtcaagggcacctttgccaca

SEQ ID NO: 214 - DNA, 21 bp, synthetic construct
  source 1..21: mol_type = other DNA
gtgggagaactggagccttca

SEQ ID NO: 215 - DNA, 21 bp, synthetic construct
  source 1..21: mol_type = other DNA
ggaactaccttgcctgctcca

SEQ ID NO: 216 - DNA, 21 bp, synthetic construct
  source 1..21: mol_type = other DNA
ggatacactgctggcttctcg

SEQ ID NO: 217 - DNA, 21 bp, synthetic construct
  source 1..21: mol_type = other DNA
gcgcttttgggattacctgcg

SEQ ID NO: 218 - DNA, 21 bp, synthetic construct
  source 1..21: mol_type = other DNA
gagcttctccggtttttccac

SEQ ID NO: 219 - DNA, 21 bp, synthetic construct
  source 1..21: mol_type = other DNA
gctttgatcaggaccagcgat

SEQ ID NO: 220 - DNA, 21 bp, synthetic construct
  source 1..21: mol_type = other DNA
gtgcttatttcgggacctggt

SEQ ID NO: 221 - DNA, 21 bp, synthetic construct
  source 1..21: mol_type = other DNA
gccaatgaggatgcgagatac

SEQ ID NO: 222 - DNA, 21 bp, synthetic construct
  source 1..21: mol_type = other DNA
ggcagcaagttcttccaaagc

SEQ ID NO: 223 - DNA, 21 bp, synthetic construct
  source 1..21: mol_type = other DNA
gacgtcgccgtccagctcgac

SEQ ID NO: 224 - DNA, 21 bp, synthetic construct
  source 1..21: mol_type = other DNA
gtcattctgttctcagttttc

SEQ ID NO: 225 - DNA, 21 bp, synthetic construct
  source 1..21: mol_type = other DNA
gccggtggtgcagatgaactt

SEQ ID NO: 226 - DNA, 26 bp, synthetic construct
  source 1..26: mol_type = other DNA
ctgccgtcctcgatgttgtggcggat

SEQ ID NO: 227 - DNA, 26 bp, synthetic construct
  source 1..26: mol_type = other DNA
cacctgggagccatttgcctctgggt

SEQ ID NO: 228 - DNA, 26 bp, synthetic construct
  source 1..26: mol_type = other DNA
caggtcacccaggaactgaggtgagt

SEQ ID NO: 229 - DNA, 26 bp, synthetic construct
  source 1..26: mol_type = other DNA
gcccctcctcagcatcttatccgagt

SEQ ID NO: 230 - DNA, 26 bp, synthetic construct
  source 1..26: mol_type = other DNA
agttgtccctgagagtcaggaggagt

SEQ ID NO: 231 - DNA, 26 bp, synthetic construct
  source 1..26: mol_type = other DNA
ccggtggtgcagatgaacttcagggt

SEQ ID NO: 232 - DNA, 26 bp, synthetic construct
  source 1..26: mol_type = other DNA
gtcgtgctgcttcatgtggtcggggt

SEQ ID NO: 233 - DNA, 26 bp, synthetic construct
  source 1..26: mol_type = other DNA
agcatcccatggaacctggagcgaat

SEQ ID NO: 234 - DNA, 26 bp, synthetic construct
  source 1..26: mol_type = other DNA
cttgccatgagccttcaccttagggt

SEQ ID NO: 235 - DNA, 26 bp, synthetic construct
  source 1..26: mol_type = other DNA
tcaagggcacctttgccacactgagt

SEQ ID NO: 236 - DNA, 26 bp, synthetic construct
  source 1..26: mol_type = other DNA
tgggagaactggagccttcagagggt

SEQ ID NO: 237 - DNA, 26 bp, synthetic construct
  source 1..26: mol_type = other DNA
gaactaccttgcctgctccagtggat

SEQ ID NO: 238 - DNA, 26 bp, synthetic construct
  source 1..26: mol_type = other DNA
gatacactgctggcttctcggagaat

SEQ ID NO: 239 - DNA, 26 bp, synthetic construct
  source 1..26: mol_type = other DNA
cgcttttgggattacctgcgctgggt

SEQ ID NO: 240 - DNA, 26 bp, synthetic construct
  source 1..26: mol_type = other DNA
agcttctccggtttttccacaagaat

SEQ ID NO: 241 - DNA, 26 bp, synthetic construct
  source 1..26: mol_type = other DNA
ctttgatcaggaccagcgatggggat

SEQ ID NO: 242 - DNA, 26 bp, synthetic construct
  source 1..26: mol_type = other DNA
tgcttatttcgggacctggttaggat

SEQ ID NO: 243 - DNA, 26 bp, synthetic construct
  source 1..26: mol_type = other DNA
ccaatgaggatgcgagataccggagt

SEQ ID NO: 244 - DNA, 26 bp, synthetic construct
  source 1..26: mol_type = other DNA
gcagcaagttcttccaaagcctggat

SEQ ID NO: 245 - DNA, 26 bp, synthetic construct
  source 1..26: mol_type = other DNA
acgtcgccgtccagctcgaccaggat

SEQ ID NO: 246 - DNA, 26 bp, synthetic construct
  source 1..26: mol_type = other DNA
tcattctgttctcagttttcctggat

SEQ ID NO: 247 - DNA, 26 bp, synthetic construct
  source 1..26: mol_type = other DNA
ccggtggtgcagatgaacttcagggt

SEQ ID NO: 248 - DNA, 21 bp, synthetic construct
  source 1..21: mol_type = other DNA
gaacagtgctcaacaggaacc

SEQ ID NO: 249 - DNA, 21 bp, synthetic construct
  source 1..21: mol_type = other DNA
gactataacagctcagttgcc

SEQ ID NO: 250 - DNA, 21 bp, synthetic construct
  source 1..21: mol_type = other DNA
gagtgttacagaaacactgcc

SEQ ID NO: 251 - DNA, 21 bp, synthetic construct
  source 1..21: mol_type = other DNA
gaagcaggtgtccttcaacaa

SEQ ID NO: 252 - DNA, 21 bp, synthetic construct
  source 1..21: mol_type = other DNA
gcttcagtttctggtggtaag

SEQ ID NO: 253 - DNA, 21 bp, synthetic construct
  source 1..21: mol_type = other DNA
gtgtaccaccaaaatcaagcc

SEQ ID NO: 254 - DNA, 21 bp, synthetic construct
  source 1..21: mol_type = other DNA
gttgtctgttgtccagagtac

SEQ ID NO: 255 - DNA, 21 bp, synthetic construct
  source 1..21: mol_type = other DNA
ggtttcaaaactgcaggctca

SEQ ID NO: 256 - DNA, 21 bp, synthetic construct
  source 1..21: mol_type = other DNA
gtttactcttcactttcacgg

SEQ ID NO: 257 - DNA, 21 bp, synthetic construct
  source 1..21: mol_type = other DNA
gagaggttctttgagtccttt

SEQ ID NO: 258 - DNA, 21 bp, synthetic construct
  source 1..21: mol_type = other DNA
gcatctttggtgtttcctatg

SEQ ID NO: 259 - DNA, 21 bp, synthetic construct
  source 1..21: mol_type = other DNA
gctgttacacatgtagttgta

SEQ ID NO: 260 - DNA, 21 bp, synthetic construct
  source 1..21: mol_type = other DNA
gtctccttaaaacatctgaga

SEQ ID NO: 261 - DNA, 21 bp, synthetic construct
  source 1..21: mol_type = other DNA
ggcttcagtagacaaatgtta

SEQ ID NO: 262 - DNA, 21 bp, synthetic construct
  source 1..21: mol_type = other DNA
gaaagaggacacatctttctt

SEQ ID NO: 263 - DNA, 21 bp, synthetic construct
  source 1..21: mol_type = other DNA
gtaccatttaccatttgccta

SEQ ID NO: 264 - DNA, 21 bp, synthetic construct
  source 1..21: mol_type = other DNA
gtcattctgttctcagttttc

SEQ ID NO: 265 - DNA, 21 bp, synthetic construct
  source 1..21: mol_type = other DNA
gaataccagtactaaaaccat

SEQ ID NO: 266 - DNA, 21 bp, synthetic construct
  source 1..21: mol_type = other DNA
gaaattaatatctttcaggac

SEQ ID NO: 267 - DNA, 21 bp, synthetic construct
  source 1..21: mol_type = other DNA
gccggtggtgcagatgaactt

SEQ ID NO: 268 - DNA, 21 bp, synthetic construct
  source 1..21: mol_type = other DNA
gacgtcgccgtccagctcgac

SEQ ID NO: 269 - DNA, 21 bp, synthetic construct
  source 1..21: mol_type = other DNA
gccggtggtgcagatgaactt

SEQ ID NO: 270 - DNA, 26 bp, synthetic construct
  source 1..26: mol_type = other DNA
aacagtgctcaacaggaaccttggat

SEQ ID NO: 271 - DNA, 26 bp, synthetic construct
  source 1..26: mol_type = other DNA
actataacagctcagttgccaagagt

SEQ ID NO: 272 - DNA, 26 bp, synthetic construct
  source 1..26: mol_type = other DNA
agtgttacagaaacactgccaagagt

SEQ ID NO: 273 - DNA, 26 bp, synthetic construct
  source 1..26: mol_type = other DNA
aagcaggtgtccttcaacaactgagt

SEQ ID NO: 274 - DNA, 26 bp, synthetic construct
  source 1..26: mol_type = other DNA
cttcagtttctggtggtaagtagagt

SEQ ID NO: 275 - DNA, 26 bp, synthetic construct
  source 1..26: mol_type = other DNA
tgtaccaccaaaatcaagcccaggat

SEQ ID NO: 276 - DNA, 26 bp, synthetic construct
  source 1..26: mol_type = other DNA
ttgtctgttgtccagagtacaaggat

SEQ ID NO: 277 - DNA, 26 bp, synthetic construct
  source 1..26: mol_type = other DNA
gtttcaaaactgcaggctcaaaggat

SEQ ID NO: 278 - DNA, 26 bp, synthetic construct
  source 1..26: mol_type = other DNA
tttactcttcactttcacggcggaat

SEQ ID NO: 279 - DNA, 26 bp, synthetic construct
  source 1..26: mol_type = other DNA
agaggttctttgagtcctttggggat

SEQ ID NO: 280 - DNA, 26 bp, synthetic construct
  source 1..26: mol_type = other DNA
catctttggtgtttcctatgatgaat

SEQ ID NO: 281 - DNA, 26 bp, synthetic construct
  source 1..26: mol_type = other DNA
ctgttacacatgtagttgtagtggat

SEQ ID NO: 282 - DNA, 26 bp, synthetic construct
  source 1..26: mol_type = other DNA
tctccttaaaacatctgagagtggat

SEQ ID NO: 283 - DNA, 26 bp, synthetic construct
  source 1..26: mol_type = other DNA
gcttcagtagacaaatgttactgaat

SEQ ID NO: 284 - DNA, 26 bp, synthetic construct
  source 1..26: mol_type = other DNA
aaagaggacacatctttctttggggt

SEQ ID NO: 285 - DNA, 26 bp, synthetic construct
  source 1..26: mol_type = other DNA
taccatttaccatttgcctacagaat

SEQ ID NO: 286 - DNA, 26 bp, synthetic construct
  source 1..26: mol_type = other DNA
tcattctgttctcagttttcctggat

SEQ ID NO: 287 - DNA, 26 bp, synthetic construct
  source 1..26: mol_type = other DNA
aataccagtactaaaaccatacgaat

SEQ ID NO: 288 - DNA, 26 bp, synthetic construct
  source 1..26: mol_type = other DNA
aaattaatatctttcaggacaggagt

SEQ ID NO: 289 - DNA, 26 bp, synthetic construct
  source 1..26: mol_type = other DNA
ccggtggtgcagatgaacttcagggt

SEQ ID NO: 290 - DNA, 26 bp, synthetic construct
  source 1..26: mol_type = other DNA
acgtcgccgtccagctcgaccaggat

SEQ ID NO: 291 - DNA, 26 bp, synthetic construct
  source 1..26: mol_type = other DNA
ccggtggtgcagatgaacttcagggt

SEQ ID NO: 292 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..24: mod_base = OTHER; note = thymine
  modified_base 27: mod_base = OTHER; note = thymine
  modified_base 32: mod_base = OTHER; note = thymine
  modified_base 51: mod_base = OTHER; note = thymine
  modified_base 56: mod_base = OTHER; note = thymine
  modified_base 60: mod_base = OTHER; note = thymine
  modified_base 63: mod_base = OTHER; note = thymine
  modified_base 68: mod_base = OTHER; note = thymine
  modified_base 70: mod_base = OTHER; note = thymine
  modified_base 72: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttagtggcctggggaaaaaggcgcagcctagactgggtgatccgatgtgtggggcgcgtcaacttgttggcgaga

SEQ ID NO: 293 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..24: mod_base = OTHER; note = thymine
  modified_base 26: mod_base = OTHER; note = thymine
  modified_base 29: mod_base = OTHER; note = thymine
  modified_base 32: mod_base = OTHER; note = thymine
  modified_base 34: mod_base = OTHER; note = thymine
  modified_base 36: mod_base = OTHER; note = thymine
  modified_base 44: mod_base = OTHER; note = thymine
  modified_base 47: mod_base = OTHER; note = thymine
  modified_base 50: mod_base = OTHER; note = thymine
  modified_base 58: mod_base = OTHER; note = thymine
  modified_base 65: mod_base = OTHER; note = thymine
  modified_base 67: mod_base = OTHER; note = thymine
  modified_base 70: mod_base = OTHER; note = thymine
  modified_base 72..73: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttgtagtactctgtaaaaaggtcctggtaccagaatgcagagtgtcgtcttgctcacgtcaacttgttggcgaga

SEQ ID NO: 294 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 25..26: mod_base = OTHER; note = thymine
  modified_base 29: mod_base = OTHER; note = thymine
  modified_base 32: mod_base = OTHER; note = thymine
  modified_base 34: mod_base = OTHER; note = thymine
  modified_base 40: mod_base = OTHER; note = thymine
  modified_base 45: mod_base = OTHER; note = thymine
  modified_base 47: mod_base = OTHER; note = thymine
  modified_base 50: mod_base = OTHER; note = thymine
  modified_base 52..54: mod_base = OTHER; note = thymine
  modified_base 65: mod_base = OTHER; note = thymine
  modified_base 70: mod_base = OTHER; note = thymine
  modified_base 73..76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgcgttagtgctgtggacatagaatctcctgtttcagcgcaaaatggggtgcttttgccgtcaacttgttggcgaga

SEQ ID NO: 295 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..25: mod_base = OTHER; note = thymine
  modified_base 29: mod_base = OTHER; note = thymine
  modified_base 44..46: mod_base = OTHER; note = thymine
  modified_base 48: mod_base = OTHER; note = thymine
  modified_base 53..54: mod_base = OTHER; note = thymine
  modified_base 61: mod_base = OTHER; note = thymine
  modified_base 65: mod_base = OTHER; note = thymine
  modified_base 69: mod_base = OTHER; note = thymine
  modified_base 72..73: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgtttgagtgcgagggaaccggatttgtcaaattcgaggctgcctgcatggttagggccgtcaacttgttggcgaga

SEQ ID NO: 296 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..22: mod_base = OTHER; note = thymine
  modified_base 24..25: mod_base = OTHER; note = thymine
  modified_base 28..29: mod_base = OTHER; note = thymine
  modified_base 32: mod_base = OTHER; note = thymine
  modified_base 34: mod_base = OTHER; note = thymine
  modified_base 39: mod_base = OTHER; note = thymine
  modified_base 41: mod_base = OTHER; note = thymine
  modified_base 45: mod_base = OTHER; note = thymine
  modified_base 52: mod_base = OTHER; note = thymine
  modified_base 65: mod_base = OTHER; note = thymine
  modified_base 69..70: mod_base = OTHER; note = thymine
  modified_base 72..73: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaactttgttgattcctctggaatatgcatcgagaatggcaaaccaaaatgcattcttcctgacgtcaacttgttggcgaga

SEQ ID NO: 297 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23: mod_base = OTHER; note = thymine
  modified_base 27: mod_base = OTHER; note = thymine
  modified_base 32: mod_base = OTHER; note = thymine
  modified_base 35: mod_base = OTHER; note = thymine
  modified_base 40..41: mod_base = OTHER; note = thymine
  modified_base 43..44: mod_base = OTHER; note = thymine
  modified_base 49: mod_base = OTHER; note = thymine
  modified_base 69..70: mod_base = OTHER; note = thymine
  modified_base 72..74: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgtgcgtggaatactgggattgttccgatgaaaaaaagggacaacgcattgtttctcacgtcaacttgttggcgaga

SEQ ID NO: 298 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..26: mod_base = OTHER; note = thymine
  modified_base 29..30: mod_base = OTHER; note = thymine
  modified_base 32: mod_base = OTHER; note = thymine
  modified_base 34: mod_base = OTHER; note = thymine
  modified_base 45: mod_base = OTHER; note = thymine
  modified_base 52: mod_base = OTHER; note = thymine
  modified_base 55: mod_base = OTHER; note = thymine
  modified_base 57..61: mod_base = OTHER; note = thymine
  modified_base 65..67: mod_base = OTHER; note = thymine
  modified_base 70: mod_base = OTHER; note = thymine
  modified_base 73..74: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttttcgttctctggaaacagaatcaacgataatatttttggatttcgtggttaccccgtcaacttgttggcgaga

SEQ ID NO: 299 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..22: mod_base = OTHER; note = thymine
  modified_base 25..26: mod_base = OTHER; note = thymine
  modified_base 31: mod_base = OTHER; note = thymine
  modified_base 34: mod_base = OTHER; note = thymine
  modified_base 40: mod_base = OTHER; note = thymine
  modified_base 45: mod_base = OTHER; note = thymine
  modified_base 50: mod_base = OTHER; note = thymine
  modified_base 53: mod_base = OTHER; note = thymine
  modified_base 58: mod_base = OTHER; note = thymine
  modified_base 63..65: mod_base = OTHER; note = thymine
  modified_base 67: mod_base = OTHER; note = thymine
  modified_base 69..70: mod_base = OTHER; note = thymine
  modified_base 73: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaactttgcttgggatgctcggcatagaatcgactcgtaggatgcgatttgtattcctgcccgcgtcaacttgttggcgaga

SEQ ID NO: 300 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..24: mod_base = OTHER; note = thymine
  modified_base 29: mod_base = OTHER; note = thymine
  modified_base 34: mod_base = OTHER; note = thymine
  modified_base 39: mod_base = OTHER; note = thymine
  modified_base 47..48: mod_base = OTHER; note = thymine
  modified_base 50: mod_base = OTHER; note = thymine
  modified_base 65..67: mod_base = OTHER; note = thymine
  modified_base 69: mod_base = OTHER; note = thymine
  modified_base 72: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttagagtacactggactaagccccttgtcgaagacgcgagactttctggtgcgtcccgtcaacttgttggcgaga

SEQ ID NO: 301 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..25: mod_base = OTHER; note = thymine
  modified_base 29: mod_base = OTHER; note = thymine
  modified_base 32: mod_base = OTHER; note = thymine
  modified_base 34: mod_base = OTHER; note = thymine
  modified_base 47: mod_base = OTHER; note = thymine
  modified_base 49..50: mod_base = OTHER; note = thymine
  modified_base 55: mod_base = OTHER; note = thymine
  modified_base 61: mod_base = OTHER; note = thymine
  modified_base 65: mod_base = OTHER; note = thymine
  modified_base 73: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttatttcagtaatctggaaacagaagctgttgaaataaggctaaatgccgcgatgataacgtcaacttgttggcgaga

SEQ ID NO: 302 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 25: mod_base = OTHER; note = thymine
  modified_base 29: mod_base = OTHER; note = thymine
  modified_base 34: mod_base = OTHER; note = thymine
  modified_base 37: mod_base = OTHER; note = thymine
  modified_base 44: mod_base = OTHER; note = thymine
  modified_base 47: mod_base = OTHER; note = thymine
  modified_base 50: mod_base = OTHER; note = thymine
  modified_base 56: mod_base = OTHER; note = thymine
  modified_base 58: mod_base = OTHER; note = thymine
  modified_base 62..63: mod_base = OTHER; note = thymine
  modified_base 65: mod_base = OTHER; note = thymine
  modified_base 67: mod_base = OTHER; note = thymine
  modified_base 70: mod_base = OTHER; note = thymine
  modified_base 75..76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgcatgggtgacctggtgagagatagtcgtaaagctgtggattatgtcgtggggttcccgtcaacttgttggcgaga

SEQ ID NO: 303 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..24: mod_base = OTHER; note = thymine
  modified_base 28: mod_base = OTHER; note = thymine
  modified_base 32: mod_base = OTHER; note = thymine
  modified_base 44: mod_base = OTHER; note = thymine
  modified_base 47: mod_base = OTHER; note = thymine
  modified_base 50: mod_base = OTHER; note = thymine
  modified_base 56: mod_base = OTHER; note = thymine
  modified_base 61..63: mod_base = OTHER; note = thymine
  modified_base 65..66: mod_base = OTHER; note = thymine
  modified_base 70: mod_base = OTHER; note = thymine
  modified_base 74: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttattgcatggctaaggggacagatcgtcctgaaactagggtttgttcggtgactcccacgtcaacttgttggcgaga

SEQ ID NO: 304 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 25..26: mod_base = OTHER; note = thymine
  modified_base 34: mod_base = OTHER; note = thymine
  modified_base 38: mod_base = OTHER; note = thymine
  modified_base 45: mod_base = OTHER; note = thymine
  modified_base 51..52: mod_base = OTHER; note = thymine
  modified_base 59: mod_base = OTHER; note = thymine
  modified_base 65: mod_base = OTHER; note = thymine
  modified_base 72..74: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttggcttgggacggtggatgcggactccccgttaaggagtggaactggcgactttgcaccgtcaacttgttggcgaga

SEQ ID NO: 305 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23: mod_base = OTHER; note = thymine
  modified_base 25: mod_base = OTHER; note = thymine
  modified_base 27..31: mod_base = OTHER; note = thymine
  modified_base 34: mod_base = OTHER; note = thymine
  modified_base 38: mod_base = OTHER; note = thymine
  modified_base 47: mod_base = OTHER; note = thymine
  modified_base 50: mod_base = OTHER; note = thymine
  modified_base 56: mod_base = OTHER; note = thymine
  modified_base 69..70: mod_base = OTHER; note = thymine
  modified_base 73: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgtgtctttttgctaaatcccgccactagtaaaactgagcacagggcattggtgataccgtcaacttgttggcgaga

SEQ ID NO: 306 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23: mod_base = OTHER; note = thymine
  modified_base 25: mod_base = OTHER; note = thymine
  modified_base 30: mod_base = OTHER; note = thymine
  modified_base 39: mod_base = OTHER; note = thymine
  modified_base 44: mod_base = OTHER; note = thymine
  modified_base 46: mod_base = OTHER; note = thymine
  modified_base 53: mod_base = OTHER; note = thymine
  modified_base 59: mod_base = OTHER; note = thymine
  modified_base 63..65: mod_base = OTHER; note = thymine
  modified_base 73: mod_base = OTHER; note = thymine
  modified_base 75..77: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgtctcaactcgcagggataggatgtaagcgatccacgtaaatttgccgacatctttgcgtcaacttgttggcgaga

SEQ ID NO: 307 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..26: mod_base = OTHER; note = thymine
  modified_base 29: mod_base = OTHER; note = thymine
  modified_base 32: mod_base = OTHER; note = thymine
  modified_base 34: mod_base = OTHER; note = thymine
  modified_base 43: mod_base = OTHER; note = thymine
  modified_base 45: mod_base = OTHER; note = thymine
  modified_base 47: mod_base = OTHER; note = thymine
  modified_base 50: mod_base = OTHER; note = thymine
  modified_base 62: mod_base = OTHER; note = thymine
  modified_base 65: mod_base = OTHER; note = thymine
  modified_base 73..74: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttcttttagtactctgggaacagtctctactaaaagaaggcataatgccgaggttatgccgtcaacttgttggcgaga

SEQ ID NO: 308 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..26: mod_base = OTHER; note = thymine
  modified_base 29: mod_base = OTHER; note = thymine
  modified_base 33..35: mod_base = OTHER; note = thymine
  modified_base 45: mod_base = OTHER; note = thymine
  modified_base 50: mod_base = OTHER; note = thymine
  modified_base 53: mod_base = OTHER; note = thymine
  modified_base 56: mod_base = OTHER; note = thymine
  modified_base 72: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttcttttagtgcgtttgcaaaggaatcaagtactagtggccaagcagcagagtacgtcacgtcaacttgttggcgaga

SEQ ID NO: 309 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 24..25: mod_base = OTHER; note = thymine
  modified_base 29: mod_base = OTHER; note = thymine
  modified_base 32: mod_base = OTHER; note = thymine
  modified_base 34: mod_base = OTHER; note = thymine
  modified_base 38: mod_base = OTHER; note = thymine
  modified_base 41..42: mod_base = OTHER; note = thymine
  modified_base 44..45: mod_base = OTHER; note = thymine
  modified_base 50: mod_base = OTHER; note = thymine
  modified_base 53..54: mod_base = OTHER; note = thymine
  modified_base 60: mod_base = OTHER; note = thymine
  modified_base 62: mod_base = OTHER; note = thymine
  modified_base 65: mod_base = OTHER; note = thymine
  modified_base 68: mod_base = OTHER; note = thymine
  modified_base 72: mod_base = OTHER; note = thymine
  modified_base 74..75: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttcgttgggtactctcaatcgttcttccactggttcaacgtctagtcctgggtcttgcccgtcaacttgttggcgaga

SEQ ID NO: 310 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..24: mod_base = OTHER; note = thymine
  modified_base 26: mod_base = OTHER; note = thymine
  modified_base 29: mod_base = OTHER; note = thymine
  modified_base 32: mod_base = OTHER; note = thymine
  modified_base 38: mod_base = OTHER; note = thymine
  modified_base 45: mod_base = OTHER; note = thymine
  modified_base 47: mod_base = OTHER; note = thymine
  modified_base 50: mod_base = OTHER; note = thymine
  modified_base 52: mod_base = OTHER; note = thymine
  modified_base 61: mod_base = OTHER; note = thymine
  modified_base 65: mod_base = OTHER; note = thymine
  modified_base 70: mod_base = OTHER; note = thymine
  modified_base 73..74: mod_base = OTHER; note = thymine
  modified_base 77: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttatagtactccgaatacggaatctagtataacaaggctaaatgccgtggttgctgcgtcaacttgttggcgaga

SEQ ID NO: 311 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..26: mod_base = OTHER; note = thymine
  modified_base 29: mod_base = OTHER; note = thymine
  modified_base 31..32: mod_base = OTHER; note = thymine
  modified_base 34: mod_base = OTHER; note = thymine
  modified_base 37: mod_base = OTHER; note = thymine
  modified_base 40: mod_base = OTHER; note = thymine
  modified_base 45: mod_base = OTHER; note = thymine
  modified_base 61: mod_base = OTHER; note = thymine
  modified_base 64..65: mod_base = OTHER; note = thymine
  modified_base 70: mod_base = OTHER; note = thymine
  modified_base 73..74: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttttcgtgttctggtagtagaatccacgaaaacaacgctagttgcggtggttatcacgtcaacttgttggcgaga

SEQ ID NO: 312 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..24: mod_base = OTHER; note = thymine
  modified_base 26..27: mod_base = OTHER; note = thymine
  modified_base 29: mod_base = OTHER; note = thymine
  modified_base 31..32: mod_base = OTHER; note = thymine
  modified_base 34: mod_base = OTHER; note = thymine
  modified_base 47: mod_base = OTHER; note = thymine
  modified_base 61: mod_base = OTHER; note = thymine
  modified_base 63: mod_base = OTHER; note = thymine
  modified_base 65: mod_base = OTHER; note = thymine
  modified_base 70: mod_base = OTHER; note = thymine
  modified_base 73..74: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttgttgtattctggcgacagaggctacaacaacaaggctgtgtgccgtggttaaaccgtcaacttgttggcgaga

SEQ ID NO: 313 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..27: mod_base = OTHER; note = thymine
  modified_base 29: mod_base = OTHER; note = thymine
  modified_base 45: mod_base = OTHER; note = thymine
  modified_base 47: mod_base = OTHER; note = thymine
  modified_base 60: mod_base = OTHER; note = thymine
  modified_base 65: mod_base = OTHER; note = thymine
  modified_base 72: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttctttttgtaaccgggaaaaaggatctaccaaaagaacgtgacatgcggggtgcatcccgtcaacttgttggcgaga

SEQ ID NO: 314 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..24: mod_base = OTHER; note = thymine
  modified_base 26: mod_base = OTHER; note = thymine
  modified_base 29: mod_base = OTHER; note = thymine
  modified_base 32: mod_base = OTHER; note = thymine
  modified_base 34: mod_base = OTHER; note = thymine
  modified_base 45: mod_base = OTHER; note = thymine
  modified_base 47: mod_base = OTHER; note = thymine
  modified_base 50: mod_base = OTHER; note = thymine
  modified_base 62: mod_base = OTHER; note = thymine
  modified_base 65: mod_base = OTHER; note = thymine
  modified_base 70: mod_base = OTHER; note = thymine
  modified_base 73..74: mod_base = OTHER; note = thymine
  modified_base 77: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttgtactactctgcaaaaggaatctagtgcaacaaggcataatgccgtggttagtacgtcaacttgttggcgaga

SEQ ID NO: 315 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..26: mod_base = OTHER; note = thymine
  modified_base 29: mod_base = OTHER; note = thymine
  modified_base 32: mod_base = OTHER; note = thymine
  modified_base 34: mod_base = OTHER; note = thymine
  modified_base 37: mod_base = OTHER; note = thymine
  modified_base 40: mod_base = OTHER; note = thymine
  modified_base 45: mod_base = OTHER; note = thymine
  modified_base 47: mod_base = OTHER; note = thymine
  modified_base 50: mod_base = OTHER; note = thymine
  modified_base 61: mod_base = OTHER; note = thymine
  modified_base 65: mod_base = OTHER; note = thymine
  modified_base 70: mod_base = OTHER; note = thymine
  modified_base 73..74: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttttagtactctcgtagtagaatctgctaaaacaaggctaaatgccgtggttgtcccgtcaacttgttggcgaga

SEQ ID NO: 316 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..26: mod_base = OTHER; note = thymine
  modified_base 34: mod_base = OTHER; note = thymine
  modified_base 41: mod_base = OTHER; note = thymine
  modified_base 45: mod_base = OTHER; note = thymine
  modified_base 47..48: mod_base = OTHER; note = thymine
  modified_base 50: mod_base = OTHER; note = thymine
  modified_base 55: mod_base = OTHER; note = thymine
  modified_base 65: mod_base = OTHER; note = thymine
  modified_base 67: mod_base = OTHER; note = thymine
  modified_base 72..74: mod_base = OTHER; note = thymine
  modified_base 77: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttattttaggaacctgggccatggatcttctaaaataagacaggatgtcgcgtttagtccgtcaacttgttggcgaga

SEQ ID NO: 317 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..25: mod_base = OTHER; note = thymine
  modified_base 29: mod_base = OTHER; note = thymine
  modified_base 32: mod_base = OTHER; note = thymine
  modified_base 34: mod_base = OTHER; note = thymine
  modified_base 45: mod_base = OTHER; note = thymine
  modified_base 47: mod_base = OTHER; note = thymine
  modified_base 50: mod_base = OTHER; note = thymine
  modified_base 62: mod_base = OTHER; note = thymine
  modified_base 65: mod_base = OTHER; note = thymine
  modified_base 70: mod_base = OTHER; note = thymine
  modified_base 73: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttctttcagtactctggagacagaatctactaaaagaaggcgtaatgccgtggtcaacacgtcaacttgttggcgaga

SEQ ID NO: 318 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..24: mod_base = OTHER; note = thymine
  modified_base 26: mod_base = OTHER; note = thymine
  modified_base 32: mod_base = OTHER; note = thymine
  modified_base 34: mod_base = OTHER; note = thymine
  modified_base 39: mod_base = OTHER; note = thymine
  modified_base 45: mod_base = OTHER; note = thymine
  modified_base 47..48: mod_base = OTHER; note = thymine
  modified_base 52: mod_base = OTHER; note = thymine
  modified_base 59: mod_base = OTHER; note = thymine
  modified_base 65: mod_base = OTHER; note = thymine
  modified_base 68: mod_base = OTHER; note = thymine
  modified_base 72: mod_base = OTHER; note = thymine
  modified_base 74: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttatgggactctgggatcagactcttccataacaaatcggaatgatgcatctatcccgtcaacttgttggcgaga

SEQ ID NO: 319 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..24: mod_base = OTHER; note = thymine
  modified_base 29: mod_base = OTHER; note = thymine
  modified_base 32: mod_base = OTHER; note = thymine
  modified_base 34: mod_base = OTHER; note = thymine
  modified_base 47: mod_base = OTHER; note = thymine
  modified_base 50: mod_base = OTHER; note = thymine
  modified_base 52: mod_base = OTHER; note = thymine
  modified_base 63: mod_base = OTHER; note = thymine
  modified_base 65: mod_base = OTHER; note = thymine
  modified_base 72..74: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttagagtactctgggaacagaacctactataacaaggcgatatgccgcgtttataccgtcaacttgttggcgaga

SEQ ID NO: 320 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..24: mod_base = OTHER; note = thymine
  modified_base 26: mod_base = OTHER; note = thymine
  modified_base 29: mod_base = OTHER; note = thymine
  modified_base 32..33: mod_base = OTHER; note = thymine
  modified_base 41: mod_base = OTHER; note = thymine
  modified_base 45: mod_base = OTHER; note = thymine
  modified_base 47: mod_base = OTHER; note = thymine
  modified_base 50: mod_base = OTHER; note = thymine
  modified_base 65: mod_base = OTHER; note = thymine
  modified_base 70: mod_base = OTHER; note = thymine
  modified_base 74: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttgtagtacttgggaaactgaatctgctacaacaacgcaaaatgcggtgagtaccccgtcaacttgttggcgaga

SEQ ID NO: 321 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..26: mod_base = OTHER; note = thymine
  modified_base 29: mod_base = OTHER; note = thymine
  modified_base 32: mod_base = OTHER; note = thymine
  modified_base 37: mod_base = OTHER; note = thymine
  modified_base 45: mod_base = OTHER; note = thymine
  modified_base 47: mod_base = OTHER; note = thymine
  modified_base 50: mod_base = OTHER; note = thymine
  modified_base 61: mod_base = OTHER; note = thymine
  modified_base 65: mod_base = OTHER; note = thymine
  modified_base 70: mod_base = OTHER; note = thymine
  modified_base 74: mod_base = OTHER; note = thymine
  modified_base 76..77: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttttagtaatgcggtcccgcaatctactaaaacaacgctgaatgcggtgggtattccgtcaacttgttggcgaga

SEQ ID NO: 322 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..26: mod_base = OTHER; note = thymine
  modified_base 29..30: mod_base = OTHER; note = thymine
  modified_base 32: mod_base = OTHER; note = thymine
  modified_base 34: mod_base = OTHER; note = thymine
  modified_base 45: mod_base = OTHER; note = thymine
  modified_base 50: mod_base = OTHER; note = thymine
  modified_base 65: mod_base = OTHER; note = thymine
  modified_base 73: mod_base = OTHER; note = thymine
  modified_base 76: mod_base = OTHER; note = thymine
  modified_base 78: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttttagttctctgggaccagaatcagctaaaacaaggcaaaatgccggggtgatgtcgtcaacttgttggcgaga

SEQ ID NO: 323 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..25: mod_base = OTHER; note = thymine
  modified_base 36: mod_base = OTHER; note = thymine
  modified_base 45..48: mod_base = OTHER; note = thymine
  modified_base 50: mod_base = OTHER; note = thymine
  modified_base 65: mod_base = OTHER; note = thymine
  modified_base 70: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgtttcagaacgccgtcagcggagttttctgaaacaaggcgaaatgccgtgggcatcccgtcaacttgttggcgaga

SEQ ID NO: 324 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..26: mod_base = OTHER; note = thymine
  modified_base 29: mod_base = OTHER; note = thymine
  modified_base 32: mod_base = OTHER; note = thymine
  modified_base 45: mod_base = OTHER; note = thymine
  modified_base 50: mod_base = OTHER; note = thymine
  modified_base 61..65: mod_base = OTHER; note = thymine
  modified_base 70: mod_base = OTHER; note = thymine
  modified_base 76..77: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttttagtgctcgggcaaccgaatccactaaaacaacgctttttgcggtgggcattacgtcaacttgttggcgaga

SEQ ID NO: 325 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..26: mod_base = OTHER; note = thymine
  modified_base 29: mod_base = OTHER; note = thymine
  modified_base 32: mod_base = OTHER; note = thymine
  modified_base 34: mod_base = OTHER; note = thymine
  modified_base 45: mod_base = OTHER; note = thymine
  modified_base 47: mod_base = OTHER; note = thymine
  modified_base 50: mod_base = OTHER; note = thymine
  modified_base 61: mod_base = OTHER; note = thymine
  modified_base 63: mod_base = OTHER; note = thymine
  modified_base 65: mod_base = OTHER; note = thymine
  modified_base 70: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttttagtactctagaaaaagagtctgctaaaacgaggctatatgccgtgggcatcccgtcaacttgttggcgaga

SEQ ID NO: 326 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..25: mod_base = OTHER; note = thymine
  modified_base 29: mod_base = OTHER; note = thymine
  modified_base 32: mod_base = OTHER; note = thymine
  modified_base 45: mod_base = OTHER; note = thymine
  modified_base 47: mod_base = OTHER; note = thymine
  modified_base 50: mod_base = OTHER; note = thymine
  modified_base 58: mod_base = OTHER; note = thymine
  modified_base 65: mod_base = OTHER; note = thymine
  modified_base 70: mod_base = OTHER; note = thymine
  modified_base 72..73: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgtttcagtactcgagccaccgaatctactgaaacagtgcgaaatgcagtcttcatcacgtcaacttgttggcgaga

SEQ ID NO: 327 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..26: mod_base = OTHER; note = thymine
  modified_base 29: mod_base = OTHER; note = thymine
  modified_base 32: mod_base = OTHER; note = thymine
  modified_base 34: mod_base = OTHER; note = thymine
  modified_base 47: mod_base = OTHER; note = thymine
  modified_base 50: mod_base = OTHER; note = thymine
  modified_base 61..63: mod_base = OTHER; note = thymine
  modified_base 65: mod_base = OTHER; note = thymine
  modified_base 70: mod_base = OTHER; note = thymine
  modified_base 73..74: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttttagtactctggaaacaggacctactaaaacaacgctttatgcggtggttatgacgtcaacttgttggcgaga

SEQ ID NO: 328 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..24: mod_base = OTHER; note = thymine
  modified_base 26: mod_base = OTHER; note = thymine
  modified_base 29: mod_base = OTHER; note = thymine
  modified_base 32: mod_base = OTHER; note = thymine
  modified_base 34: mod_base = OTHER; note = thymine
  modified_base 47..50: mod_base = OTHER; note = thymine
  modified_base 52: mod_base = OTHER; note = thymine
  modified_base 62: mod_base = OTHER; note = thymine
  modified_base 65: mod_base = OTHER; note = thymine
  modified_base 70: mod_base = OTHER; note = thymine
  modified_base 73: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttatactactctggaaacggaaccttttataacaaggcataatgccgtggtcaaaccgtcaacttgttggcgaga

SEQ ID NO: 329 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23: mod_base = OTHER; note = thymine
  modified_base 26: mod_base = OTHER; note = thymine
  modified_base 29: mod_base = OTHER; note = thymine
  modified_base 32: mod_base = OTHER; note = thymine
  modified_base 34: mod_base = OTHER; note = thymine
  modified_base 37: mod_base = OTHER; note = thymine
  modified_base 39: mod_base = OTHER; note = thymine
  modified_base 47: mod_base = OTHER; note = thymine
  modified_base 49..50: mod_base = OTHER; note = thymine
  modified_base 61: mod_base = OTHER; note = thymine
  modified_base 65: mod_base = OTHER; note = thymine
  modified_base 67: mod_base = OTHER; note = thymine
  modified_base 70: mod_base = OTHER; note = thymine
  modified_base 72..73: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgtcgtagtactctcgtgtgagaacctattacgacaagactacgtgtcgtgttcatcacgtcaacttgttggcgaga

SEQ ID NO: 330 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..24: mod_base = OTHER; note = thymine
  modified_base 26: mod_base = OTHER; note = thymine
  modified_base 28..29: mod_base = OTHER; note = thymine
  modified_base 34: mod_base = OTHER; note = thymine
  modified_base 36: mod_base = OTHER; note = thymine
  modified_base 45: mod_base = OTHER; note = thymine
  modified_base 47: mod_base = OTHER; note = thymine
  modified_base 50: mod_base = OTHER; note = thymine
  modified_base 62: mod_base = OTHER; note = thymine
  modified_base 65: mod_base = OTHER; note = thymine
  modified_base 68: mod_base = OTHER; note = thymine
  modified_base 70: mod_base = OTHER; note = thymine
  modified_base 73: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttctattaccctgtaaagcggatctagtagaacaaagcataatgctgtagtcatgacgtcaacttgttggcgaga

SEQ ID NO: 331 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..26: mod_base = OTHER; note = thymine
  modified_base 29: mod_base = OTHER; note = thymine
  modified_base 34..36: mod_base = OTHER; note = thymine
  modified_base 43: mod_base = OTHER; note = thymine
  modified_base 45: mod_base = OTHER; note = thymine
  modified_base 47: mod_base = OTHER; note = thymine
  modified_base 50: mod_base = OTHER; note = thymine
  modified_base 58: mod_base = OTHER; note = thymine
  modified_base 63: mod_base = OTHER; note = thymine
  modified_base 65: mod_base = OTHER; note = thymine
  modified_base 70..73: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttttagtacactttaacgagtatctactaaaacaatgcggtatgcagttttcatcacgtcaacttgttggcgaga

SEQ ID NO: 332 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..26: mod_base = OTHER; note = thymine
  modified_base 29: mod_base = OTHER; note = thymine
  modified_base 32: mod_base = OTHER; note = thymine
  modified_base 34: mod_base = OTHER; note = thymine
  modified_base 45: mod_base = OTHER; note = thymine
  modified_base 47: mod_base = OTHER; note = thymine
  modified_base 62..63: mod_base = OTHER; note = thymine
  modified_base 65: mod_base = OTHER; note = thymine
  modified_base 68: mod_base = OTHER; note = thymine
  modified_base 70: mod_base = OTHER; note = thymine
  modified_base 72..73: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttttcgtactctggagacggaatctacgaaaacaaagcattatgctgtgttcagcccgtcaacttgttggcgaga

SEQ ID NO: 333 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..25: mod_base = OTHER; note = thymine
  modified_base 29: mod_base = OTHER; note = thymine
  modified_base 32: mod_base = OTHER; note = thymine
  modified_base 47: mod_base = OTHER; note = thymine
  modified_base 49..50: mod_base = OTHER; note = thymine
  modified_base 65: mod_base = OTHER; note = thymine
  modified_base 67: mod_base = OTHER; note = thymine
  modified_base 72..73: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgtttgagtactcgggccaccgaacctattcaaacaagacaaaatgtcgcgttcatcacgtcaacttgttggcgaga

SEQ ID NO: 334 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 24: mod_base = OTHER; note = thymine
  modified_base 26: mod_base = OTHER; note = thymine
  modified_base 29: mod_base = OTHER; note = thymine
  modified_base 32: mod_base = OTHER; note = thymine
  modified_base 34: mod_base = OTHER; note = thymine
  modified_base 47: mod_base = OTHER; note = thymine
  modified_base 50: mod_base = OTHER; note = thymine
  modified_base 62: mod_base = OTHER; note = thymine
  modified_base 65: mod_base = OTHER; note = thymine
  modified_base 68: mod_base = OTHER; note = thymine
  modified_base 70: mod_base = OTHER; note = thymine
  modified_base 72..73: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgctgtagtactctggaaacagaacctgctacagcaaagcatgatgctgtgttcatgccgtcaacttgttggcgaga

SEQ ID NO: 335 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..26: mod_base = OTHER; note = thymine
  modified_base 29: mod_base = OTHER; note = thymine
  modified_base 32: mod_base = OTHER; note = thymine
  modified_base 34..35: mod_base = OTHER; note = thymine
  modified_base 38: mod_base = OTHER; note = thymine
  modified_base 41: mod_base = OTHER; note = thymine
  modified_base 45: mod_base = OTHER; note = thymine
  modified_base 47: mod_base = OTHER; note = thymine
  modified_base 49: mod_base = OTHER; note = thymine
  modified_base 55: mod_base = OTHER; note = thymine
  modified_base 65: mod_base = OTHER; note = thymine
  modified_base 70: mod_base = OTHER; note = thymine
  modified_base 73..74: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttattttggtactcttgatagtgaatctatcaaaataaggcaaaatgccgtggttatcccgtcaacttgttggcgaga

SEQ ID NO: 336 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..24: mod_base = OTHER; note = thymine
  modified_base 26: mod_base = OTHER; note = thymine
  modified_base 29: mod_base = OTHER; note = thymine
  modified_base 32: mod_base = OTHER; note = thymine
  modified_base 34..35: mod_base = OTHER; note = thymine
  modified_base 45: mod_base = OTHER; note = thymine
  modified_base 47: mod_base = OTHER; note = thymine
  modified_base 50: mod_base = OTHER; note = thymine
  modified_base 52: mod_base = OTHER; note = thymine
  modified_base 58: mod_base = OTHER; note = thymine
  modified_base 61: mod_base = OTHER; note = thymine
  modified_base 65: mod_base = OTHER; note = thymine
  modified_base 70: mod_base = OTHER; note = thymine
  modified_base 72..73: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttatactactcttgaaagggaatctagtataacagtgctgaatgcagtgttcatgccgtcaacttgttggcgaga

SEQ ID NO: 337 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..26: mod_base = OTHER; note = thymine
  modified_base 29: mod_base = OTHER; note = thymine
  modified_base 32: mod_base = OTHER; note = thymine
  modified_base 34: mod_base = OTHER; note = thymine
  modified_base 36: mod_base = OTHER; note = thymine
  modified_base 47: mod_base = OTHER; note = thymine
  modified_base 50: mod_base = OTHER; note = thymine
  modified_base 58: mod_base = OTHER; note = thymine
  modified_base 65: mod_base = OTHER; note = thymine
  modified_base 72..73: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttttactactctgtgaacggaacctagtaaaacaatgcacgatgcagcgttcatcccgtcaacttgttggcgaga

SEQ ID NO: 338 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..26: mod_base = OTHER; note = thymine
  modified_base 32: mod_base = OTHER; note = thymine
  modified_base 34: mod_base = OTHER; note = thymine
  modified_base 39: mod_base = OTHER; note = thymine
  modified_base 47: mod_base = OTHER; note = thymine
  modified_base 50: mod_base = OTHER; note = thymine
  modified_base 65: mod_base = OTHER; note = thymine
  modified_base 67: mod_base = OTHER; note = thymine
  modified_base 72..73: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttttaggactctggaataagaacctcctaaaacaagacaagatgtcgcgttcatcacgtcaacttgttggcgaga

SEQ ID NO: 339 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..26: mod_base = OTHER; note = thymine
  modified_base 29: mod_base = OTHER; note = thymine
  modified_base 32: mod_base = OTHER; note = thymine
  modified_base 34: mod_base = OTHER; note = thymine
  modified_base 45: mod_base = OTHER; note = thymine
  modified_base 47: mod_base = OTHER; note = thymine
  modified_base 50: mod_base = OTHER; note = thymine
  modified_base 65: mod_base = OTHER; note = thymine
  modified_base 70: mod_base = OTHER; note = thymine
  modified_base 72..74: mod_base = OTHER; note = thymine
  modified_base 76: mod_base = OTHER; note = thymine
  modified_base 78: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttttagtactctggaaacagaatctactaaaacaaggcaaaatgccgtgtttatctcgtcaacttgttggcgaga

SEQ ID NO: 340 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..24: mod_base = OTHER; note = thymine
  modified_base 26: mod_base = OTHER; note = thymine
  modified_base 29: mod_base = OTHER; note = thymine
  modified_base 32: mod_base = OTHER; note = thymine
  modified_base 35: mod_base = OTHER; note = thymine
  modified_base 45: mod_base = OTHER; note = thymine
  modified_base 49..50: mod_base = OTHER; note = thymine
  modified_base 65: mod_base = OTHER; note = thymine
  modified_base 73: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttgtagtgctcgtgcaaccgaatccattacaacaacgcaaaatgcggcggtcatcacgtcaacttgttggcgaga

SEQ ID NO: 341 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..24: mod_base = OTHER; note = thymine
  modified_base 26: mod_base = OTHER; note = thymine
  modified_base 29: mod_base = OTHER; note = thymine
  modified_base 32: mod_base = OTHER; note = thymine
  modified_base 35: mod_base = OTHER; note = thymine
  modified_base 40: mod_base = OTHER; note = thymine
  modified_base 45: mod_base = OTHER; note = thymine
  modified_base 47: mod_base = OTHER; note = thymine
  modified_base 50: mod_base = OTHER; note = thymine
  modified_base 61: mod_base = OTHER; note = thymine
  modified_base 65: mod_base = OTHER; note = thymine
  modified_base 70: mod_base = OTHER; note = thymine
  modified_base 73: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttctagtactcgtggaatggaatctactagaacaacgctgaatgcggtgctcatcccgtcaacttgttggcgaga

SEQ ID NO: 342 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..24: mod_base = OTHER; note = thymine
  modified_base 26: mod_base = OTHER; note = thymine
  modified_base 29: mod_base = OTHER; note = thymine
  modified_base 32: mod_base = OTHER; note = thymine
  modified_base 34: mod_base = OTHER; note = thymine
  modified_base 45: mod_base = OTHER; note = thymine
  modified_base 47: mod_base = OTHER; note = thymine
  modified_base 50: mod_base = OTHER; note = thymine
  modified_base 65: mod_base = OTHER; note = thymine
  modified_base 68: mod_base = OTHER; note = thymine
  modified_base 70: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttgtagtactctgcaaacagaatctactacaacagagcaaaatgctgtggacatcacgtcaacttgttggcgaga

SEQ ID NO: 343 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..26: mod_base = OTHER; note = thymine
  modified_base 29: mod_base = OTHER; note = thymine
  modified_base 32: mod_base = OTHER; note = thymine
  modified_base 34: mod_base = OTHER; note = thymine
  modified_base 39: mod_base = OTHER; note = thymine
  modified_base 45: mod_base = OTHER; note = thymine
  modified_base 47: mod_base = OTHER; note = thymine
  modified_base 50: mod_base = OTHER; note = thymine
  modified_base 61: mod_base = OTHER; note = thymine
  modified_base 65: mod_base = OTHER; note = thymine
  modified_base 72..73: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttttagtactgtggactcacaatctactaaaacaacgctgaatgcggcattcatcccgtcaacttgttggcgaga

SEQ ID NO: 344 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 24: mod_base = OTHER; note = thymine
  modified_base 26: mod_base = OTHER; note = thymine
  modified_base 32: mod_base = OTHER; note = thymine
  modified_base 34: mod_base = OTHER; note = thymine
  modified_base 45: mod_base = OTHER; note = thymine
  modified_base 47..48: mod_base = OTHER; note = thymine
  modified_base 50: mod_base = OTHER; note = thymine
  modified_base 52: mod_base = OTHER; note = thymine
  modified_base 54: mod_base = OTHER; note = thymine
  modified_base 62: mod_base = OTHER; note = thymine
  modified_base 65: mod_base = OTHER; note = thymine
  modified_base 72..73: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgatgtaggactctggaaacagaatcttctatatcaacgcgtgatgcggcgttcatcccgtcaacttgttggcgaga

SEQ ID NO: 345 - RNA, 97 nt, synthetic construct
  modified_base 6: mod_base = OTHER; note = thymine
  modified_base 9: mod_base = OTHER; note = thymine
  modified_base 15: mod_base = OTHER; note = thymine
  modified_base 20..21: mod_base = OTHER; note = thymine
  modified_base 23..24: mod_base = OTHER; note = thymine
  modified_base 29: mod_base = OTHER; note = thymine
  modified_base 32: mod_base = OTHER; note = thymine
  modified_base 34: mod_base = OTHER; note = thymine
  modified_base 37: mod_base = OTHER; note = thymine
  modified_base 44: mod_base = OTHER; note = thymine
  modified_base 47: mod_base = OTHER; note = thymine
  modified_base 50: mod_base = OTHER; note = thymine
  modified_base 65: mod_base = OTHER; note = thymine
  modified_base 72: mod_base = OTHER; note = thymine
  modified_base 74: mod_base = OTHER; note = thymine
  modified_base 76: mod_base = OTHER; note = thymine
  modified_base 81: mod_base = OTHER; note = thymine
  modified_base 86..87: mod_base = OTHER; note = thymine
  modified_base 89..90: mod_base = OTHER; note = thymine
  misc_feature 1..97: note = gRNA
  source 1..97: mol_type = other RNA
gccggtggtgcagatgaacttgttggactactctgataacagatcctagtccaacaacgcagaatgcggcgtctatcacgtcaacttgttggcgaga

SEQ ID NO: 346 - DNA, 117 bp, synthetic construct
  source 1..117: mol_type = other DNA
aaaagagcacgagatggcagannnnnnnnnnnnnnnnnnnnnnnnnnnnnnnnnnnnnnnnnnnnnnnnnnnnnnnnnnnnnnnnnnnnnnnnnnnntcacgtgctcagtctgggcc

SEQ ID NO: 347 - DNA, 40 bp, synthetic construct
  source 1..40: mol_type = other DNA
ggtaatacgactcactatagggcccagactgagcacgtga

SEQ ID NO: 348 - DNA, 127 bp, synthetic construct
  modified_base 12..21: mod_base = OTHER; note = Locked nucleic acids
  source 1..127: mol_type = other DNA
aaaagagcacgaaaaaaaaaaagatggcagannnnnnnnnnnnnnnnnnnnnnnnnnnnnnnnnnnnnnnnnnnnnnnnnnnnnnnnnnnnnnnnnnnnnnnnnnnntcacgtgctcagtctgggcc

SEQ ID NO: 349 - DNA, 14 bp, synthetic construct
  modified_base 3: mod_base = OTHER; note = Locked nucleic acid
  modified_base 7..9: mod_base = OTHER; note = Locked nucleic acid
  modified_base 13: mod_base = OTHER; note = Locked nucleic acid
  source 1..14: mol_type = other DNA
tgccatctcgtgct

SEQ ID NO: 350 - DNA, 20 bp, synthetic construct
  source 1..20: mol_type = other DNA
ccggtggtgcagatgaactt

SEQ ID NO: 351 - DNA, 20 bp, synthetic construct
  source 1..20: mol_type = other DNA
aagttcatctgcaccaccgg

SEQ ID NO: 352 - DNA, 43 bp, synthetic construct
  source 1..43: mol_type = other DNA
ggggataatacgactcactatagccggtggtgcagatgaactt

SEQ ID NO: 353 - DNA, 19 bp, synthetic construct
  source 1..19: mol_type = other DNA
gcatgtcgtcaagcaacac

SEQ ID NO: 354 - DNA, 20 bp, synthetic construct
  source 1..20: mol_type = other DNA
gctgtgtatgtccaagtgtg

SEQ ID NO: 355 - DNA, 189 bp, synthetic construct
  source 1..189: mol_type = other DNA
aagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgcatgtcgtcaagcaacac

SEQ ID NO: 356 - DNA, 100 bp, synthetic construct
  source 1..100: mol_type = other DNA
acaacgccctgaatgacctgaacaacctggtcatcaccagggatgaaaacgagaaactggaatactatgagaagttccagcacacttggacatacacagc

SEQ ID NO: 357 - DNA, 57 bp, synthetic construct
  source 1..57: mol_type = other DNA
gttttagtactctggaaacagaatctactaaaacaangcnnnatgcngtggttatcc

SEQ ID NO: 358 - DNA, 56 bp, synthetic construct
  source 1..56: mol_type = other DNA
gttttagtactctggaaacagaatctactaaaacaaggcaaaatgccgtgttatct

SEQ ID NO: 359 - DNA, 57 bp, synthetic construct
  source 1..57: mol_type = other DNA
gttttagtactctgtcgaaagaatctgctaaaacaaggccttgtgccgtagtcgcgc

SEQ ID NO: 360 - DNA, 57 bp, synthetic construct
  source 1..57: mol_type = other DNA
gttttagtactctcgtagtagaatctgctaaaacaaggctaaatgccgtggttgtcc

SEQ ID NO: 361 - DNA, 57 bp, synthetic construct
  source 1..57: mol_type = other DNA
gttttagtactctagaaaaagagtctgctaaaacgaggctatatgccgtgggcatcc

SEQ ID NO: 362 - DNA, 57 bp, synthetic construct
  source 1..57: mol_type = other DNA
gttgtaatactctggaaacagaatctgctacaacaaggctatatgccgtgcgtatac

SEQ ID NO: 363 - DNA, 57 bp, synthetic construct
  source 1..57: mol_type = other DNA
cgttgggtactctcaatcgttcttccactggttcaacgtctagtcctgggtcttgcc

SEQ ID NO: 364 - DNA, 57 bp, synthetic construct
  source 1..57: mol_type = other DNA
ggcttgggacggtggatgcggactccccgttaaggagtggaactggcgactttgcac

SEQ ID NO: 365 - DNA, 57 bp, synthetic construct
  source 1..57: mol_type = other DNA
gtgtctttttgctaaatcccgccactagtaaaactgagcacagggcattggtgatac

SEQ ID NO: 366 - DNA, 57 bp, synthetic construct
  source 1..57: mol_type = other DNA
gtttcagaacgccgtcagcggagttttctgaaacaaggcgaaatgccgtgggcatcc

SEQ ID NO: 367 - DNA, 57 bp, synthetic construct
  source 1..57: mol_type = other DNA
gtttacggactcttaagtcagaagcttcgtaaacaaggcaaaatgccgtgtgcatca

SEQ ID NO: 368 - DNA, 57 bp, synthetic construct
  source 1..57: mol_type = other DNA
gtttcagcactctgtaaaaggaatctactgaaacaatgctagatgcagcgttcatgc

SEQ ID NO: 369 - DNA, 57 bp, synthetic construct
  source 1..57: mol_type = other DNA
gttttggtactctgtgatcggaatctaccaaaacaatgcgatatgcagcgtttatgc

SEQ ID NO: 370 - DNA, 57 bp, synthetic construct
  source 1..57: mol_type = other DNA
gatgtaggactctggaaacagaatcttctatatcaacgcgtgatgcggcgttcatcc

SEQ ID NO: 371 - DNA, 57 bp, synthetic construct
  source 1..57: mol_type = other DNA
gctgtagtactctggaaacagaacctgctacagcaaagcatgatgctgtgttcatgc

SEQ ID NO: 372 - DNA, 57 bp, synthetic construct
  source 1..57: mol_type = other DNA
gttgtaatactttggtaacttagcctattacaacaatgcggaatgcaggctctatcc

SEQ ID NO: 373 - DNA, 57 bp, synthetic construct
  source 1..57: mol_type = other DNA
gttggactactctgataacagatcctagtccaacaacgcagaatgcggcgtctatca

SEQ ID NO: 374 - DNA, 57 bp, synthetic construct
  source 1..57: mol_type = other DNA
ctttttgtaaccgggaaaaaggatctaccaaaagaacgtgacatgcggggtgcatcc

SEQ ID NO: 375 - DNA, 57 bp, synthetic construct
  source 1..57: mol_type = other DNA
cttttattaatcgataaaaagaagctaataaaagaaagcttgatgctgtggttatcc

SEQ ID NO: 376 - DNA, 57 bp, synthetic construct
  source 1..57: mol_type = other DNA
gtcttagtactgttgaatgaacatctgctaagacaaagcttaatgctgtgggtatca

SEQ ID NO: 377 - DNA, 57 bp, synthetic construct
  source 1..57: mol_type = other DNA
gttgtagtactcttgattggggatctactacaacaaagctttatgctgaagttgtcc

SEQ ID NO: 378 - DNA, 57 bp, synthetic construct
  source 1..57: mol_type = other DNA
gttttagtaatgcggtcccgcaatctactaaaacaacgctgaatgcggtgggtattc

SEQ ID NO: 379 - DNA, 57 bp, synthetic construct
  source 1..57: mol_type = other DNA
gtttgagtacttgtctttgggaatctactcaaataacgcgaaatgcggtgggtatcc

SEQ ID NO: 380 - DNA, 57 bp, synthetic construct
  source 1..57: mol_type = other DNA
tttttagtactctgggaacggaatctactaaaataaagcgaaatgctgtggttatcc

SEQ ID NO: 381 - DNA, 57 bp, synthetic construct
  source 1..57: mol_type = other DNA
gttttcgttctctggaaacagaatcaacgataatatttttggatttcgtggttaccc

SEQ ID NO: 382 - DNA, 57 bp, synthetic construct
  source 1..57: mol_type = other DNA
gttttagtgctcgggcaaccgaatccactaaaacaacgctttttgcggtgggcatta

SEQ ID NO: 383 - DNA, 57 bp, synthetic construct
  source 1..57: mol_type = other DNA
gttatagtactcgggaacccgaatctactataacaaggcattatgccgtggttacta

SEQ ID NO: 384 - DNA, 56 bp, synthetic construct
  source 1..56: mol_type = other DNA
gtttagtactctggaaacaggacctactaaaacaacgctttatgcggtggttatga

SEQ ID NO: 385 - DNA, 57 bp, synthetic construct
  source 1..57: mol_type = other DNA
gttttagtactctggaaacngaatctactaaaacaangcaaaatgcngtgttcatcn

SEQ ID NO: 386 - DNA, 57 bp, synthetic construct
  source 1..57: mol_type = other DNA
gttttagtactctggaaacagaatctactaaaacaaggcaaaatgccgtgtttatct

SEQ ID NO: 387 - DNA, 57 bp, synthetic construct
  source 1..57: mol_type = other DNA
ggttttagtactctggaaacagaatctactaaaacaaggcaaaatgccgtgtttatc

SEQ ID NO: 388 - DNA, 57 bp, synthetic construct
  source 1..57: mol_type = other DNA
cttttagtactccgctaacggattctactaaaagaagcgagaatgcggtgagcatac

SEQ ID NO: 389 - DNA, 57 bp, synthetic construct
  source 1..57: mol_type = other DNA
cttttagtactctggaaacggaatcttctaaaagaatgcgatatgcagtgttcatgc

SEQ ID NO: 390 - DNA, 57 bp, synthetic construct
  source 1..57: mol_type = other DNA
gttctagtactcgtggaatggaatctactagaacaacgctgaatgcggtgctcatcc

SEQ ID NO: 391 - DNA, 57 bp, synthetic construct
  source 1..57: mol_type = other DNA
gttatactactcttgaaagggaatctagtataacagtgctgaatgcagtgttcatgc

SEQ ID NO: 392 - DNA, 57 bp, synthetic construct
  source 1..57: mol_type = other DNA
gttttactactctgtgaacggaacctagtaaaacaatgcacgatgcagcgttcatcc

SEQ ID NO: 393 - DNA, 57 bp, synthetic construct
  source 1..57: mol_type = other DNA
gttttagtactgtggactcacaatctactaaaacaacgctgaatgcggcattcatcc

SEQ ID NO: 394 - DNA, 57 bp, synthetic construct
  source 1..57: mol_type = other DNA
gttttagtactctggaaacagaatctactaaaacagagcagaatgctgtgttcatgc

SEQ ID NO: 395 - DNA, 57 bp, synthetic construct
  source 1..57: mol_type = other DNA
gttgtgatacccagccagctggatctaccacaacaacgcaaaatgcggggttcgtca

SEQ ID NO: 396 - DNA, 57 bp, synthetic construct
  source 1..57: mol_type = other DNA
gttgtagtgctcgtgcaaccgaatccattacaacaacgcaaaatgcggcggtcatca

SEQ ID NO: 397 - DNA, 56 bp, synthetic construct
  source 1..56: mol_type = other DNA
gttctattaccctgaaagcggatctagtagaacaaagcataatgctgtagtcatga

SEQ ID NO: 398 - DNA, 57 bp, synthetic construct
  source 1..57: mol_type = other DNA
gttgtagtactctgcaaacagaatctactacaacagagcaaaatgctgtggacatca

SEQ ID NO: 399 - DNA, 57 bp, synthetic construct
  source 1..57: mol_type = other DNA
gttgtccgactctataaattgaatcttggacaacaacgcaaaatgcggtgctcatcc

SEQ ID NO: 400 - DNA, 57 bp, synthetic construct
  source 1..57: mol_type = other DNA
gttgtaggactctgaacacagaaccttctacaacaagccaaaatggcgtgctcatag

SEQ ID NO: 401 - DNA, 57 bp, synthetic construct
  source 1..57: mol_type = other DNA
gttaacgtactctgctgacagaatctacgttaacaaagcgaagtgctgcgttcatca

SEQ ID NO: 402 - DNA, 57 bp, synthetic construct
  source 1..57: mol_type = other DNA
gttttcgtactctggagacggaatctacgaaaacaaagcattatgctgtgttcagcc

SEQ ID NO: 403 - DNA, 57 bp, synthetic construct
  source 1..57: mol_type = other DNA
gtttcagtactcgagccaccgaatctactgaaacagtgcgaaatgcagtcttcatca

SEQ ID NO: 404 - DNA, 57 bp, synthetic construct
  source 1..57: mol_type = other DNA
gttttagtacactttaacgagtatctactaaaacaatgcggtatgcagttttcatca

SEQ ID NO: 405 - DNA, 57 bp, synthetic construct
  source 1..57: mol_type = other DNA
gtttcagtactccgtagttggaacctactgaaacaaggctatatgccggggtcataa

SEQ ID NO: 406 - DNA, 57 bp, synthetic construct
  source 1..57: mol_type = other DNA
gttttagtactctgtcgccagaatctactaaaacaacgctatatgcggtggttatca

SEQ ID NO: 407 - DNA, 57 bp, synthetic construct
  source 1..57: mol_type = other DNA
gtcgtagtactctcgtgtgagaacctattacgacaagactacgtgtcgtgttcatca

SEQ ID NO: 408 - DNA, 57 bp, synthetic construct
  source 1..57: mol_type = other DNA
gttttaggactctggaataagaacctcctaaaacaagacaagatgtcgcgttcatca

SEQ ID NO: 409 - DNA, 57 bp, synthetic construct
  source 1..57: mol_type = other DNA
gtttgagtactcgggccaccgaacctattcaaacaagacaaaatgtcgcgttcatca

SEQ ID NO: 410 - DNA, 57 bp, synthetic construct
  source 1..57: mol_type = other DNA
gtttcagtactcacggaacagaatctactgaaacaagacaagatgtcgcattcatca

SEQ ID NO: 411 - DNA, 57 bp, synthetic construct
  source 1..57: mol_type = other DNA
gttttactactctggtaacagaacctgctaaaacaaggcaatatgccgtgtgcatcc

SEQ ID NO: 412 - DNA, 57 bp, synthetic construct
  source 1..57: mol_type = other DNA
gttttagtactctggaaacagaatctactaaaacaaggcaaaatgccgtgtttatca

SEQ ID NO: 413 - DNA, 40 bp, synthetic construct
  source 1..40: mol_type = other DNA
ctacggcagagaacccagagtacctgggtctggacgtgcc

SEQ ID NO: 414 - DNA, 40 bp, synthetic construct
  source 1..40: mol_type = other DNA
atcttgtctgttgtccagagtacaaggatttcagctctcc

SEQ ID NO: 415 - DNA, 40 bp, synthetic construct
  source 1..40: mol_type = other DNA
gttaataccagtactaaaaccatacgaattattggtttat